(19) United States
(12) Patent Application Publication
(10) Pub. No.: US 2025/0017940 A1
(43) Pub. Date: Jan. 16, 2025

(54) SELECTIVE PEPTIDOMIMETIC MODULATORS OF CAV2.2 (N-TYPE) VOLTAGE-GATED CALCIUM CHANNELS AND USES THEREOF

(71) Applicants: New York University, New York, NY (US); University of Pittsburgh - Of the Commonwealth System of Higher Education, Pittsburgh, PA (US)

(72) Inventors: Rajesh KHANNA, Short Hills, NJ (US); Carlos J. CAMACHO, Pittsburgh, PA (US)

(73) Assignees: New York University, New York, NY (US); University of Pittsburgh - Of the Commonwealth System of Higher Education, Pittsburgh, PA (US)

(21) Appl. No.: 18/758,804

(22) Filed: Jun. 28, 2024

Related U.S. Application Data
(60) Provisional application No. 63/524,386, filed on Jun. 30, 2023.

(51) Int. Cl. A61K 31/5377; A61K 31/155; A61K 31/18; A61K 31/404; A61K 31/437; A61K 31/4402; A61K 31/443; A61K 31/4439; A61K 31/444; A61K 31/4709; A61K 31/498; A61K 31/519; A61K 31/55; A61P 25/04; C07C 279/14; C07C 311/19; C07D 207/16; C07D 209/08; C07D 211/14; C07D 213/74; C07D 215/06; C07D 401/12; C07D 401/14; C07D 405/12; C07D 413/12; C07D 413/14; C07D 417/12; C07D 417/14; C07D 471/04; C07D 487/04; C07D 498/04; G01N 33/68; G16B 15/30

(52) U.S. Cl. CPC A61K 31/5377; A61K 31/155; A61K 31/18; A61K 31/404; A61K 31/437; A61K 31/4402; A61K 31/443; A61K 31/4439; A61K 31/444; A61K 31/4709; A61K 31/498; A61K 31/519; A61K 31/55; A61P 25/04; C07C 279/14; C07C 311/19; C07D 207/16; C07D 209/08; C07D 211/14; C07D 213/74; C07D 215/06; C07D 401/12; C07D 401/14; C07D 405/12; C07D 413/12; C07D 413/14; C07D 417/12; C07D 417/14; C07D 471/04; C07D 487/04; C07D 498/04; G01N 33/6872; G16B 15/30; G01N 2333/4703

(57) ABSTRACT

The present invention relates to compounds of Formula (I):

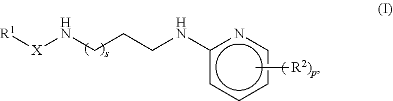

where R¹, R², X, s, and p are as defined herein. The present invention also relates to compounds of Formula (II), Formula (III), and:

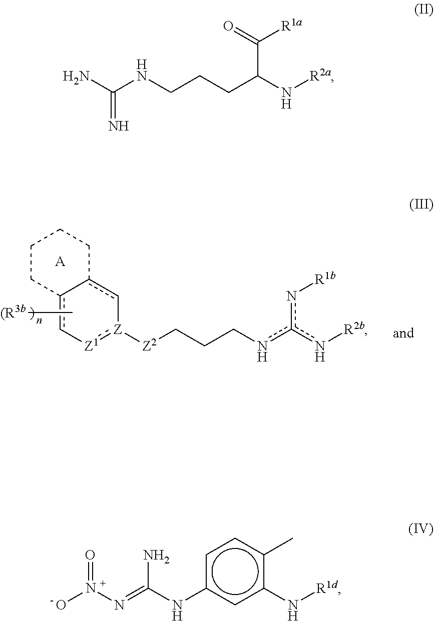

where R^{1a}, R^{1b}, R^{1d}, R^{2a}, R^{2b}, R^{3b}, X, Z, Z¹, Z², s, and p are as defined herein. Methods of using these compounds to regulate Cav2.2.target double-stranded RNA and to treat diseases and disorders, e.g., chronic pain, are also disclosed.

### CROSS-REFERENCE TO RELATED APPLICATIONS

This application claims priority to U.S. Provisional Application No. 63/524,386, filed Jun. 30, 2023, the disclosure of which is herein incorporated by reference in its entirety.

### STATEMENT REGARDING FEDERALLY SPONSORED RESEARCH OR DEVELOPMENT

This invention was made with government support under R01 GM097082 awarded by the National Institutes of Health. The government has certain rights in the invention.

### FIELD OF INVENTION

The present invention relates to rationally designed selective peptidomimetic modulators of CaV2.2 (N-type) voltage-gated calcium channels for chronic pain.

### BACKGROUND OF THE INVENTION

In the central and peripheral nervous systems, transmembrane Caᵥ2.2 (N-type) voltage-gated calcium channels are expressed in the dorsal root ganglia (DRG) and spinal dorsal horn (SDH)—two important sites for nociceptive transmission (Hoppanova & Lacinova, 2022). Within the spinal cord, Caᵥ2.2 channels are localized in the central terminals of primary afferent fibers where they mediate the release of excitatory neurotransmitters (Heinke et al., 2004). Knockout mouse models of Caᵥ2.2 (Hatakeyama et al., 2001; Kim et al., 2001) demonstrated a key role for these channels in nociceptive pathways: Caᵥ2.2 deficient mice show reduced response to mechanical stimuli in the von Frey test and increased tail flick latency in response to radiant heat, indicating altered spinal reflexes (Irwin et al., 1951); however, in the hot plate test, which is an assay of supraspinal nociceptive integration (Giglio, et al., 2006), pain responses were unaltered (Kim et al., 2001). The expression and function of Caᵥ2.2 channels increases following nerve injury (Cizkova et al., 2002; Yang et al., 2018; Yu et al., 2019b) demonstrating plasticity of this target for therapeutic intervention.

Three drugs targeting Caᵥ2.2 channels are commercially available for management of neuropathic pain conditions. Ziconotide (Prialt®)—a synthetic version of the cone snail toxin ω-conotoxin MVIIA—is a Caᵥ2.2 channel blocker and was the first non-opioid intrathecal analgesic approved by the US Food and Drug Administration for the treatment of intractable chronic pain (Doggrell, 2004). Its use is hampered by its invasive route of administration, narrow therapeutic window, and a panoply of side effects. Gabapentin (Neurontin®) and Pregabalin (Lyrica®)—ligands of α2δ-1 auxiliary subunit of Caᵥ2.2 channels—alleviate chronic pain by disrupting Caᵥ2.2-α2δ-1 interaction to prevent Caᵥ2.2 trafficking to the plasma membrane (Bauer et al., 2010; Hendrich et al., 2008; Sutton et al., 2002). Both gabapentinoids have low efficacy and present with serious side effects (Evoy et al., 2021). Misuse of gabapentinoids has led to an increase in overdose-related deaths between 2019 and 2020 (Kuehn, 2022). Therefore, there is a critical need to develop novel medicines that effectively manage pain without producing negative side effects.

Alternative approaches have been devised to regulate the functional activity of Caᵥ2.2 channels by targeting proteins that interact with them. In this regard, collapsin response mediator protein 2 (CRMP2) was identified as a regulator of Caᵥ2.2 trafficking and function (Brittain et al., 2009; Chi et al., 2009; Khanna et al., 2019). CRMP2 is a microtubule-binding protein that regulates neuronal polarity in vitro (Goshima et al., 1995; Inagaki et al., 2001). Importantly, CRMP2 interacts with Caᵥ2.2 (Brittain et al., 2009; Khanna et al., 2007). Overexpression of CRMP2 leads to enhanced Caᵥ2.2 currents and surface expression (Brittain et al., 2009; Chi et al., 2009) and enhanced neurotransmitter release (Brittain et al., 2009; Chi et al., 2009) in hippocampal (Brittain et al. 2009) and DRG neurons (Chi et al., 2009). A 15-amino-acid peptide (designated CBD3, for calcium channel binding domain 3) generated from CRMP2 interfered with the Caᵥ2.2-CRMP2 protein-protein interaction and decreased calcium influx, transmitter release, and acute, inflammatory, and neuropathic pain (Brittain et al., 2011b). Homology-guided mutational analysis of CBD3 revealed an antinociceptive core in the first six amino acids with two residues (Ala₁ and Arg₄) accounting for most of the binding affinity (Moutal et al., 2018).

Thus, there exists an unmet need for modulators of CaV2.2 (N-type) voltage-gated calcium channels, and in particular selective modulators of CaV2.2 that can be used to treat chronic pain.

### SUMMARY OF THE INVENTION

Various non-limiting aspects and embodiments of the invention are described below.

In one aspect, the present disclosure provides a compound of Formula (I):

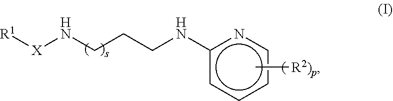

wherein
X is a covalent bond or —C(O)—;
p is 0, 1, 2, 3, or 4;
s is 1 or 2;
R¹ is selected from the group consisting of C₁₋₆ alkyl, benzyl, —NR³R⁴, monocyclic or bicyclic heteroaryl, and monocyclic or bicyclic heterocyclyl, wherein C₁₋₆ alkyl, benzyl, monocyclic or bicyclic heteroaryl, and monocyclic or bicyclic heterocyclyl can be optionally substituted from 1 to 3 times with R⁵;
R² is independently at each occurrence a C₁₋₆ alkyl;
R³ is H;
R⁴ is selected from the group consisting of —H, C₁₋₆ alkyl, C₃₋₈ cycloalkyl, monocyclic or bicyclic aryl, hereroaryl, heterocyclyl, —CH₂-heterocyclyl, and —CH₂-heteroaryl, wherein C₁₋₆ alkyl, monocyclic or bicyclic aryl, heteroaryl, heterocyclyl, —CH₂-heterocyclyl, and —CH₂— heteroaryl can be optionally substituted from 1 to 3 times with R⁶; or
R³ and R⁴ combine with the nitrogen atom to which they are attached to form a non-aromatic heterocyclyl, wherein the non-aromatic heterocyclyl can be optionally substituted from 1 to 3 times with a substituent selected independently from the group consisting of —H, C₁₋₆ alkyl, and —NH—C(O)—Me;
R⁵ is selected from the group consisting of —H, halogen, —OH, phenyl, C₁₋₆ alkyl, benzyl, —OC₁₋₆ alkyl, ═O, C₃₋₈ cycloalkyl, monocyclic heteroaryl, wherein C₁₋₆ alkyl and C₃₋₈ cycloalkyl can be optionally substituted from 1 to 3 times with —NH₂;
R⁶ is independently selected from the group consisting of —H, —NMe₂, C₁₋₆ alkyl, —O—C₁₋₆ alkyl, heteroaryl, —CH₂-heterocyclyl, —CH₂-heteroaryl, heterocyclyl, —OPh, C₃₋₈ cycloalkyl, ═O, and heteroaryl, wherein —CH₂-heterocyclyl, —CH₂-heteroaryl, heterocyclyl, heteroaryl, and —OPh can be optionally substituted from 1 to 3 times with a substituent selected independently from the group consisting of —H, C₁₋₆ alkyl, C₃₋₈ cycloalkyl, —OH, ═O, halogen, and —SC₁₋₆ alkyl,
or a solvate, or a pharmaceutically acceptable salt thereof.

In another aspect, the present disclosure provides a compound of Formula (II):

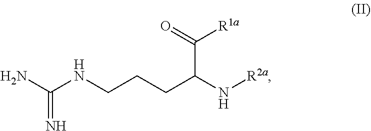

wherein
R^{1a} is selected from the group consisting of —OH, —OC₁₋₆ alkyl, and —NHR^{3a};
R^{2a} is selected from the group consisting of —H, —C(O)—R^{4a}, —C(Y)C₁₋₆ alkyl, —C(Y)C₃₋₈ cycloalkyl, —C(O)OC₁₋₆ alkyl, —C(Y)aryl, —C(Y)NH-aryl, —C(Y)NH—CH₂-aryl, —C(Y)NHC₃₋₈ cycloalkyl, and —S(O)₂aryl, wherein —C(O)—R^{4a}, —C(Y)C₁₋₆ alkyl, —C(Y)C₃₋₈ cycloalkyl, —C(O)OC₁₋₆ alkyl, —C(Y)aryl, —C(Y)NH-aryl, —C(Y)NH—CH₂-aryl, —C(Y)NHC₃₋₈ cycloalkyl, and —S(O)₂aryl can be optionally substituted from 1 to 3 times with a substituent selected independently from C₁₋₆ alkyl and —NH₂,
R^{3a} is selected from C₁₋₆ alkyl or monocyclic or bicyclic aryl, wherein C₁₋₆ alkyl and monocyclic and bicyclic aryl can be optionally substituted from 1 to 3 times with a substituent selected independently from —COOH and —NO₂;
R^{4a} is non-aromatic heterocyclyl optionally substituted with —C(O)OC₁₋₆ alkyl;
Y is O or S,
or a solvate, or a pharmaceutically acceptable salt thereof.

In another aspect, the present disclosure provides a compound of Formula (III):

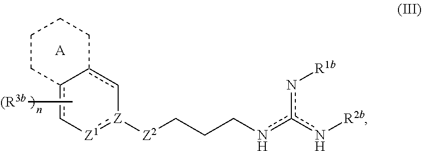

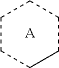

is optional, and if present is

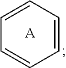

[symbol] is a single or a double bond;
Z is C or N;
Z¹ is CH, CH₂, N, or a covalent bond;
Z² is CH₂ or a covalent bond;
n is 0, 1, or 2;
R^{1b} is selected from the group consisting of —H, C₁₋₆ alkyl, C₃₋₈ cycloalkyl, aryl, and benzyl, wherein C₁₋₆ alkyl, aryl, and benzyl can be optionally substituted with a substituent selected from —CN, —OC₁₋₆ alkyl, heteroaryl, and non-aromatic heterocyclyl;
R^{2b} is selected from the group consisting of —H, C₁₋₆ alkyl, C₃₋₈ cycloalkyl, aryl, and benzyl, wherein C₁₋₆ alkyl, aryl, and benzyl can be optionally substituted with a substituent selected from —CN, —OC₁₋₆ alkyl, heteroaryl, and non-aromatic heterocyclyl;
R^{3b} is —H or C₁₋₆ alkyl,
or a solvate, or a pharmaceutically acceptable salt thereof.

In another aspect, the present disclosure provides a compound of Formula (IV):

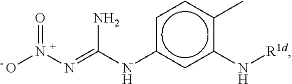

wherein
R^{1d} is —COOC₁₋₆ alkyl, —C(O)aryl, or —C(O)heteroaryl, wherein —C(O)aryl can be optionally substituted with —NO₂,
or a solvate, or a pharmaceutically acceptable salt thereof.

In another aspect, the present disclosure provides a compound selected from the group consisting of

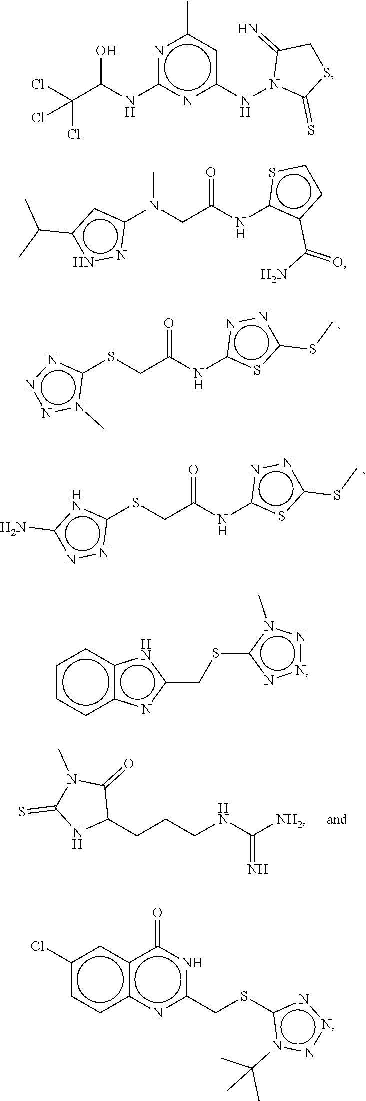

or a solvate, or a pharmaceutically acceptable salt thereof.

In another aspect, the present disclosure provides a method of regulating Cav2.2. This method comprises contacting Cav2.2 with the compound according to the disclosure, the composition comprising any compound according to the present disclosure, or the dosage form comprising any compound according to the present disclosure.

In another aspect, the present disclosure provides a method of treating, reducing or alleviating pain in an individual in need of such treatment. This method comprises administering to the individual an effective amount of the compound according to the disclosure, the composition comprising any compound according to the present disclosure, or the dosage form comprising any compound according to the present disclosure.

In another aspect, the present disclosure provides a method of identifying one or more compounds capable of regulating Cav2.2. This method comprises providing a CBD3 peptide and identifying a stable structural motif of the CBD3 peptide. This method further comprises providing one or more pharmacophore models and mapping the stable structural motif of the CBD3 peptide or its properties into said pharmacophore models. This method further comprises providing one or more compounds and screening each of the pharmacophore models against these compounds. This method further comprises selecting one or more compounds that have the highest Cav2.2 recognition score for future testing.

These and other aspects of the present invention will become apparent to those skilled in the art after a reading of the following detailed description of the invention, including the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show stability analysis of dipeptides within CBD3 and pharmacophore model on average cluster center of A₁R₂ dipeptide and sample of matched hits. FIG. 1A shows largest cluster of MD snapshots with RMSD less than 1 Å for dipeptides A₁R₂, R₂S₃, S₃R₄, and R₄L₅, also shown is average percentage of cluster size in 3 independent simulations. FIG. 1B shows RMSD relative to cluster center and times the amino acid side chains are free of contacts (3.8 Å for hydrogen bond donors and 4 Å for hydrophobic side chains) within 3 ns windows in a representative MD trajectory of CBD3 peptide. Gray regions highlight regions for which RMSD of snapshots are less than 1 Å from cluster center, and side chains are 80% or more free of contacts within 3 ns windows. FIG. 1C shows pharmacophores radiuses used: guanidine group, 0.78 Å for positive ion and 1 Å for hydrogen bond donor; 0.78 Å for other hydrogen bond donors, and 1.0 Å for hydrophobic atoms. FIG. 1D shows sample of compounds that matched all pharmacophores using ZincPharmer.

FIGS. 2A-2C show compound screening using depolarization-induced Ca²⁺ influx and whole-cell patch-clamp in DRG neurons identify various high-voltage-activated Ca²⁺ channels inhibitors. FIG. 2A shows percent change in average response of DRG sensory neurons incubated overnight with 20 μM of the indicated compounds in response to 90 mM KCl. N=61-629 cells; error bars indicate mean±SEM. p values as indicated. One-Way ANOVA with the Dunnett post hoc test. Only the significances for the compounds that inhibit Ca²⁺ influx more than 50% are shown in the plot. FIG. 2B shows representative calcium current traces recorded from small- to medium-sized DRGs incubated overnight with 0.1% DMSO or 20 μM of test compounds as indicated in the figure. Currents were evoked by 200-ms pulse between −70 and +60 mV. FIG. 2C shows summary of bar graph showing the normalized peak total ICa²⁺ density. N=16-98 cells indicated in parenthesis; error bars indicate mean±SEM; p values as indicated; Kruskal-Wallis test followed by Dunn's post hoc test.

FIGS. 3A-3D show that CBD3063 reduces total calcium currents in DRG neurons. FIG. 3A shows representative calcium current traces recorded from small- to medium-sized DRGs incubated overnight with 20 μM of CBD3063 as indicated in the figure. Currents were evoked by 200-ms pulse between −70 and +60 mV. FIG. 3B shows Double Boltzmann fits for current density-voltage curve. Asterisk (*) indicate p<0.05; Multiple Mann-Whitney tests. FIG. 3C shows summary of bar graph showing peak calcium current densities (pA/pF). p value as indicated; Mann-Whitney test. FIG. 3D shows Boltzmann fits for voltage-dependent activation and inactivation as shown. Half-maximal activation potential of activation and inactivation (V_{1/2}) and slope values (k) for activation and inactivation are presented in Table 3. N=13−16 cells; error bars indicate mean±SEM.

FIGS. 4A-4D show that CBD3063 suppresses Caᵥ2.2-CRMP2 interaction and surface trafficking of the channel. FIG. 4A shows representative immunoblots. FIG. 4B shows summary of CRMP2 immunoprecipitation (IP) to detect Caᵥ2.2 from CAD cells treated overnight with CBD3063 (20 μM) (n=3). p value as indicated; Unpaired t-test. FIG. 4C shows representative micrographs of DRG cells immunolabeled with Caᵥ2.2. FIG. 4D shows summary of Caᵥ2.2 membrane/cytosol ratio in DRG neurons treated overnight with 20 μM of CBD3063 (n=25−38). Error bars show mean±SEM; p values as indicated; Mann-Whitney test.

FIGS. 5A-5E show that N-type (Caᵥ2.2) calcium currents are reduced by CBD3063 in DRG neurons. FIG. 5A represents summary of bar graph showing the normalized peak ICa²⁺ density after incubating sensory neurons with DMSO (0.1%), 2, 20 and 50 μM of CBD3063. N=13-30 cells; error bars indicate mean±SEM; p values as indicated; Kruskal-Wallis test followed by Dunn's post hoc test. FIG. 5B shows representative N-type calcium current traces recorded from small- to medium-sized DRGs incubated overnight with 20 μM of CBD3063 as indicated in the figure. Currents were evoked by 200-ms pulse between −70 and +60 mV.

FIG. 5C shows Double Boltzmann fits for current density-voltage curve. Asterisk (*) indicate p<0.05; Multiple Mann-Whitney tests. FIG. 5D represents summary of bar graph showing peak N-type calcium current densities (pA/pF). p value as indicated; Mann-Whitney test. FIG. 5E shows Boltzmann fits for voltage-dependent activation and inactivation kinetics as shown. Half-maximal activation potential of activation and inactivation (V_{1/2}) and slope values (k) for activation and inactivation are presented in Table 3. N=12-17 cells.

FIGS. 6A-6H show that CBD3063 does not inhibit other voltage-gated calcium channels. FIGS. 6A, 6C, 6E, and 6G show Double Boltzmann fits for L-, P/Q-, R—, and T-type current density-voltage curves, respectively. Multiple Mann-Whitney tests. FIGS. 6B, 6D, 6F, and 6H represent summary of bar graph showing peak L-, P/Q-, R—, and T-type calcium current densities (pA/pF). p value as indicated; Mann-Whitney test. N=7-11 cells; Error bars indicate mean±SEM; Half-maximal activation potential (V_{1/2}) and slope values (k) for activation and inactivation are presented in Table 3.

FIGS. 7A-7D show that sensory neuron excitability is decreased by CBD3063. FIG. 7A shows representative traces in response to the indicated current injection steps from rat DRG neurons treated 0.1% DMSO (control; black circles) or 20 μM CBD3063 (cyan squares). FIG. 7B shows quantification of resting membrane potential in millivolts (mV) in the two conditions. FIG. 7C shows quantification of the rheobase in the presence of DMSO or 20 μM CBD3063. FIG. 7D shows summary of the number of evoked action potentials in response to current injection between 0-120 pA. N=6 cells; p value as indicated; Mann-Whitney test (FIGS. 7B and 7C) and Multiple Mann-Whitney test. Error bars indicate mean±SEM.

FIGS. 8A-8D show results from single and repeated intrathecal injection of CBD3063 (0.3 μg/kg) reverse mechanical allodynia following spared nerve injury. FIG. 8A shows a timeline of the experimental paradigm indicating that pre-SNI baseline measurements of withdrawal threshold were taken before nerve injury. A single intrathecal injection of CBD3063 was applied 7 days after SNI, and withdrawal thresholds were followed from 0.5 hour to 36 hours post injection. p value as indicated; Multiple Mann-Whitney tests. FIG. 8B shows quantification of the area under the curve between 0.5 and 36 hours after i.t. injection. p value as indicated; Mann-Whitney test. N=6-10 rats per conditions; error bars indicate mean±SEM. FIG. 8C shows timeline of the repeated i.t. injection of CBD3063 indicating that pre-SNI baseline measurements of withdrawal threshold were taken before nerve injury. After assessing the paw withdrawal threshold 7 days after SNI, intrathecal injections of CBD3063 were applied for 14 consecutive days and paw withdrawal thresholds were assessed at 1, 3, 5, 7 10, and 14 days after the first i.t. injection. p value as indicated; Multiple Mann-Whitney tests. FIG. 8D shows quantification of the area under the curve between SNI and 14 days after first i.t. injection. p value as indicated; Mann-Whitney test. N=5 rats per group; error bars indicate mean±SEM.

FIGS. 9A-9B show cluster centers for three independent simulations of the PEP96 and TAT-ARSRLA. The clusters are based on A₁R₂(PEP96) (FIG. 9A) and A₁₂R₁₃ (TAT-ARSRLA) (FIG. 9B) with less than 1 Å from cluster center.

FIG. 10 shows full list of predicted structures.

FIG. 11A shows A₁R₂ cluster center highlighting three pharmacophores present in all compounds except CBD3026. FIG. 11B shows structures of compounds obtained from the ZincPharmer screen which were found to inhibit Ca²⁺ influx by more than 50% (FIGS. 2A-C). Dashed circle shows guanidine group (arginine).

FIGS. 12A-12F show that CBD3063 does not affect CRMP2 phosphorylation. FIGS. 12A, 12C, and 12E show representative immunoblots. FIGS. 12B, 12D, and 12F show quantitative analysis of total and phosphorylated CRMP2 at the indicated kinase target sites from CAD cells treated overnight with 0.1% DMSO (as control) or 20 μM CBD3063 (n=4). Error bars show mean±SEM; p values as indicated; Mann-Whitney test.

FIGS. 13A-13D show that total sodium currents in DRG neurons are not affected by CBD3063. FIG. 13A shows representative sodium current traces recorded from small- to medium-sized DRGs incubated overnight with 20 μM of CBD3063 as indicated in the figure. Currents were evoked by 150-ms pulse between −70 and +60 mV. FIG. 13B shows Double Boltzmann fits for current density-voltage curve. No statistical significance was observed after applying a Multiple Mann-Whitney tests. FIG. 13C shows summary of bar graph showing peak sodium current densities (pA/pF). p value as indicated; Mann-Whitney test. FIG. 13D shows Boltzmann fits for voltage-dependent activation and inactivation as shown. Half-maximal activation potential of activation and inactivation (V_{1/2}) and slope values (k) for activation and inactivation are presented in Table 3. N=9-12 cells; error bars indicate mean±SEM.

FIGS. 14A-14C show that CBD3063 does not affect voltage-gated potassium channels. FIG. 14A shows representative total potassium current traces recorded from sensory neurons in the presence of 0.1% DMSO or 20 μM CBD3063. Currents were evoked by 300-ms pulse between −80 and +60 mV. FIG. 14B shows Double Boltzmann fits for current density-voltage curves. FIG. 14C shows summary of peak current densities (pA/pF). N=9−10 cells; error bars indicate mean±SEM; p values as indicated; Mann-Whitney test.

FIG. 15 shows a novel molecular dynamics approach that was developed and applied to identify the Cav2.2 recognition motif of the core CBD3 peptide as the A1R2 dipeptide. Its presenting motif was used to design pharmacophore models to screen 27 million compounds in the open access server ZincPharmer. Of 200 curated hits, 77 compounds were assessed using depolarization-evoked calcium influx in rat dorsal root ganglion (DRG) neurons. Nine compounds were tested using electrophysiology and one compound (CBD3063) was evaluated biochemically, electrophysiologically, and behaviorally effects in a model of experimental pain.

### DETAILED DESCRIPTION

Transmembrane Cav2.2 (N-type) voltage-gated calcium channels are genetically and pharmacologically validated pain targets. Clinical block of Cav2.2 (e.g., with Prialt) or indirect modulation (e.g., with gabapentinoids) mitigates chronic pain but is constrained by side effects. The cytosolic auxiliary subunit collapsin response mediator protein 2 (CRMP2) targets Cav2.2 to the sensory neuron membrane and regulates their function. A CRMP2-derived peptide (CBD3) uncouples the Cav2.2-CRMP2 interaction to inhibit calcium influx, transmitter release and pain. Homology-guided mutagenesis of CBD3 revealed an antinociceptive core at A1RSR4. In the present disclosure, the A1R2 CBD3 dipeptide was identified as critical for Cav2.2 molecular recognition and served as a scaffold for identification of small molecule peptidomimetic allosteric regulators of Cav2.2.

In the absence of any significant structural information on Caᵥ2.2 to guide a rational design of small molecule inhibitors, a novel pipeline was developed that leveraged molecular dynamics of the core peptide and identified the A₁R2 dipeptide as the most stable conformational motif. Based on well-established biophysical principles (Rajamani et al., 2004), this motif was defined to be the anchor of the interaction responsible for molecular recognition of Caᵥ2.2. Thus, the A₁R₂ ensemble was used as a scaffold to design pharmacophore models to predict first-in-class compounds to disrupt CRMP2-Caᵥ2.2 interaction. Screening 27 million commercially available compounds led to the identification of a first-in-class peptidomimetic ((3R)-3-acetamido-N-[3-(pyridin-2-ylamino)propyl]piperidine-1-carboxamide; hereafter designated as CBD3063) that disrupted the Caᵥ2.2-CRMP2 interaction, inhibited Caᵥ2.2 function, and reversed experimental neuropathic pain.

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 1-20 carbon atoms in its backbone (e.g., C₁-C₂₀ for straight chain, C₂-C₂₀ for branched chain), and alternatively, about 1-10 carbon atoms, or about 1 to 6 carbon atoms. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, binaphthyl, anthracyi and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone of as part of a larger moiety, e.g., “heteroaralkyl,” or “heteroaralkoxy,” refer to groups having 5 to 10 ring atoms (i.e., monocyclic or bicyclic), in some embodiments 5, 6, 9, or 10 ring atoms. In some embodiments, such rings have 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quatemized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic, tricyclic, tetracyclic, and/or otherwise polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “non-aromatic heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, tricyclic, tetracyclic, and/or otherwise polycyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

The term “unsaturated,” as used herein, means that a moiety has one or more units of unsaturation.

The term “halogen” means F, Cl, Br, or I; the term “halide” refers to a halogen radical or substituent, namely—F, —Cl, —Br, or —I.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this invention.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Unless otherwise stated, all crystalline forms of the compounds of the invention and salts thereof are also within the scope of the invention. The compounds of the invention may be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the invention are anhydrous and non-solvated. By “anhydrous” is meant that the crystalline form of the compound contains essentially no bound water in the crystal lattice structure, i.e., the compound does not form a crystalline hydrate.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

The term “pharmaceutically acceptable salts” means the relatively non-toxic, inorganic, and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like (see, for example, Berge et al., “Pharmaceutical Salts,” J. Pharm. Sci., 66:1-9 (1977) and Remington's Pharmaceutical Sciences, 17th ed., Mack Publishing Company, Easton, Pa., 1985, p. 1418, which are hereby incorporated by reference in their entirety). Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include, for example, sodium hydride, sodium hydroxide, potassium hydroxide, calcium hydroxide, aluminium hydroxide, lithium hydroxide, magnesium hydroxide, and zinc hydroxide. Suitable amine base addition salts are prepared from amines which have sufficient basicity to form a stable salt, and preferably include those amines which are frequently used in medicinal chemistry because of their low toxicity and acceptability for medical use, such as ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylarnine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, dicyclohexylamine, and the like.

The term “solvate” refers to a compound of Formula (I), Formula (IA), Formula (IB), Formula (II), Formula (III), Formula (IIIA), Formula (IIIB), Formula (IIIC), Formula (IIID), Formula (IIIE), and Formula (IV) in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

In some embodiments, the compounds of the invention are substantially isolated. By “substantially isolated” is meant that a particular compound is at least partially isolated from impurities. For example, in some embodiments a compound of the invention comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances.

### Compounds of the Invention

In one aspect, the present disclosure provides a compound of Formula (I):

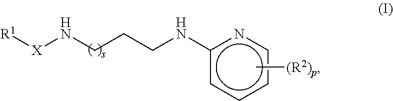

wherein
X is a covalent bond or —C(O)—;
p is 0, 1, 2, 3, or 4;
s is 1 or 2;
R¹ is selected from the group consisting of C₁₋₆ alkyl, benzyl, —NR³R⁴, monocyclic or bicyclic heteroaryl, and monocyclic or bicyclic heterocyclyl, wherein C₁₋₆ alkyl, benzyl, monocyclic or bicyclic heteroaryl, and monocyclic or bicyclic heterocyclyl can be optionally substituted from 1 to 3 times with R⁵;
R² is independently at each occurrence a C₁₋₆ alkyl;
R³ is H;
R⁴ is selected from the group consisting of —H, C₁₋₆ alkyl, C₃₋₈ cycloalkyl, monocyclic or bicyclic aryl, hereroaryl, heterocyclyl, —CH₂-heterocyclyl, and —CH₂-heteroaryl, wherein C₁₋₆ alkyl, monocyclic or bicyclic aryl, heteroaryl, heterocyclyl, —CH₂-heterocyclyl, and —CH₂— heteroaryl can be optionally substituted from 1 to 3 times with R⁶; or
R³ and R⁴ combine with the nitrogen atom to which they are attached to form a non-aromatic heterocyclyl, wherein the non-aromatic heterocyclyl can be optionally substituted from 1 to 3 times with a substituent selected independently from the group consisting of —H, C₁₋₆ alkyl, and —NH—C(O)—Me;
R⁵ is selected from the group consisting of —H, halogen, —OH, phenyl, C₁₋₆ alkyl, benzyl, —OC₁₋₆ alkyl, ═O, C₃₋₈ cycloalkyl, monocyclic heteroaryl, wherein C₁₋₆ alkyl and C₃₋₈ cycloalkyl can be optionally substituted from 1 to 3 times with —NH₂;
R⁶ is independently selected from the group consisting of —H, —NMe₂, C₁₋₆ alkyl, —O—C₁₋₆ alkyl, heteroaryl, —CH₂-heterocyclyl, —CH₂-heteroaryl, heterocyclyl, —OPh, C₃₋₈ cycloalkyl, ═O, and heteroaryl, wherein —CH₂-heterocyclyl, —CH₂-heteroaryl, heterocyclyl, heteroaryl, and —OPh can be optionally substituted from 1 to 3 times with a substituent selected independently from the group consisting of —H, C₁₋₆ alkyl, C₃₋₈ cycloalkyl, —OH, ═O, halogen, and —SC₁₋₆ alkyl,
or a solvate, or a pharmaceutically acceptable salt thereof.

In some embodiments, R¹ is selected from the group consisting of

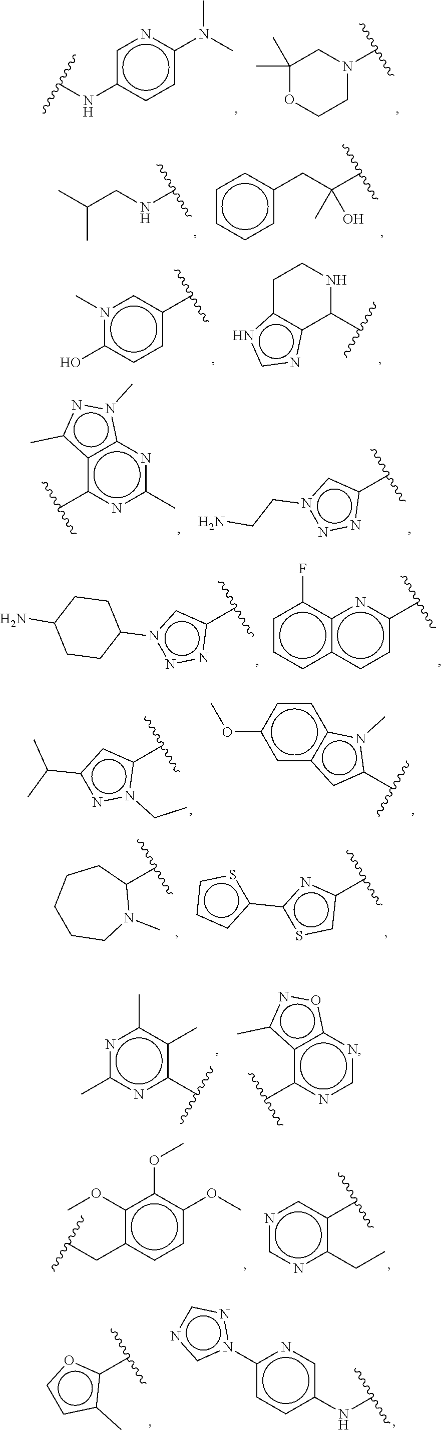

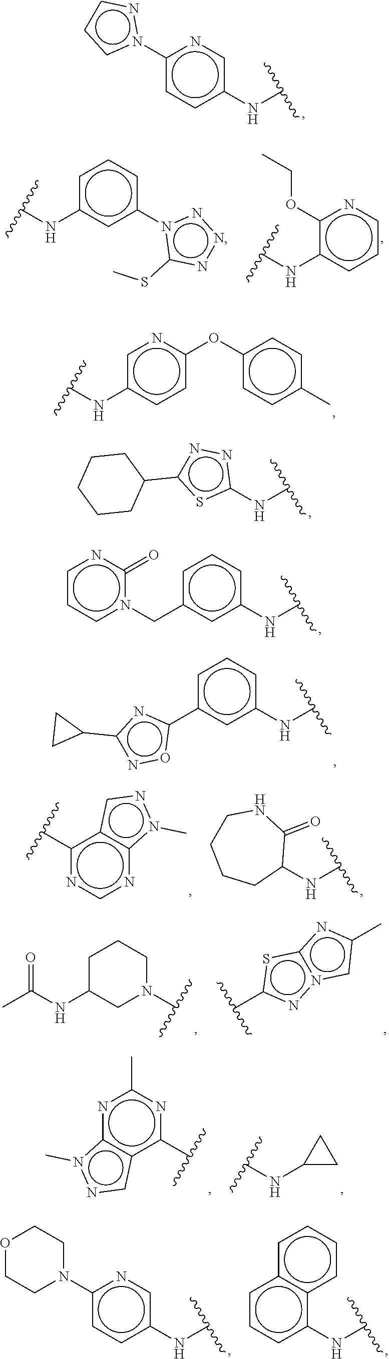

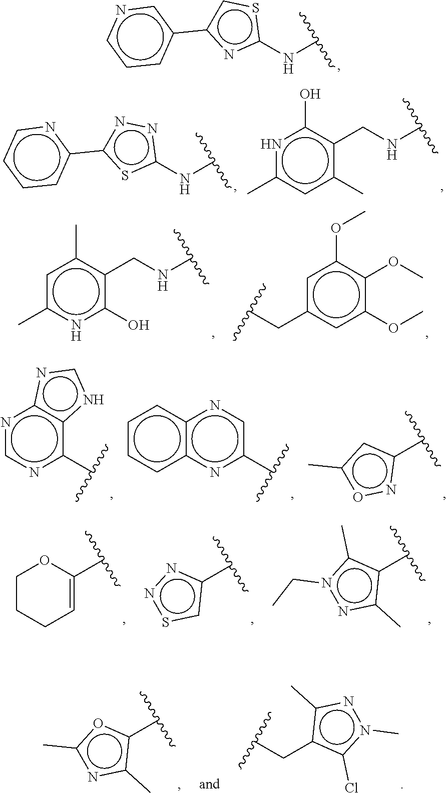

In some embodiments, R² is —H or —Me.

In some embodiments, R³ and R⁴ combine with the nitrogen atom to which they are attached to form morpholinyl or piperidinyl, wherein the morpholinyl or the piperidinyl can be optionally substituted from 1 to 3 times with H, C₁₋₆ alkyl, or —NH—C(O)—Me.

In some embodiments, the compound of Formula (I) has a Formula (IA):

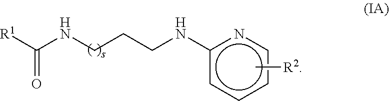

In some embodiments, the compound of Formula (I) has a Formula (IB):

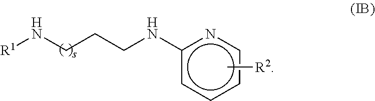

In some embodiments, the compound of Formula (1) has a Formula (I)

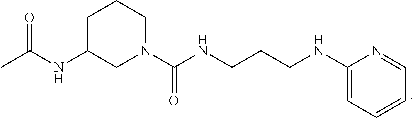 [SMILES: CC(=O)NC1CCCN(C(=O)NCCCNc2ccccn2)C1]

In some embodiments, the compound of Formula (I) is selected from a group consisting of

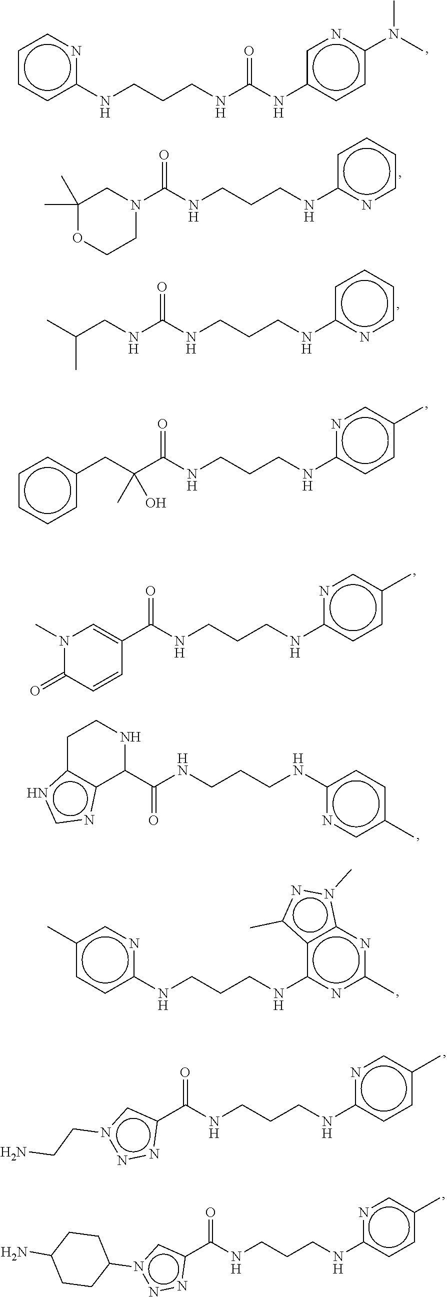

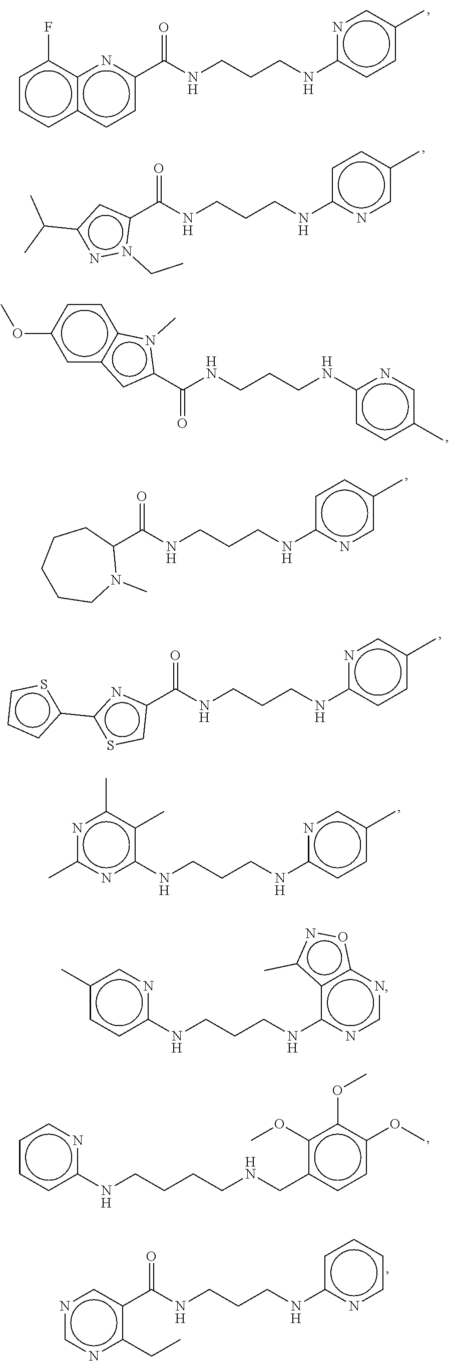

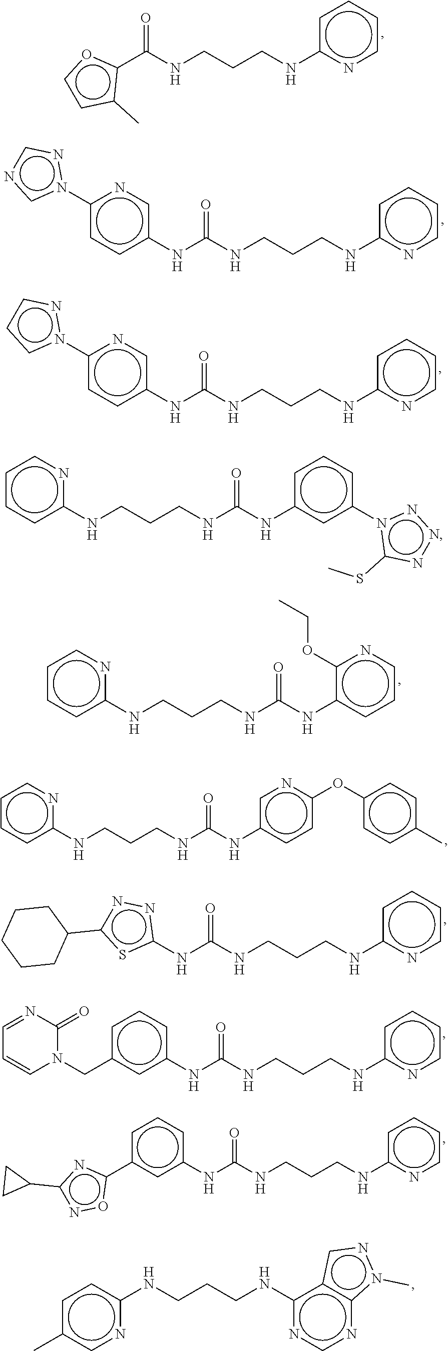

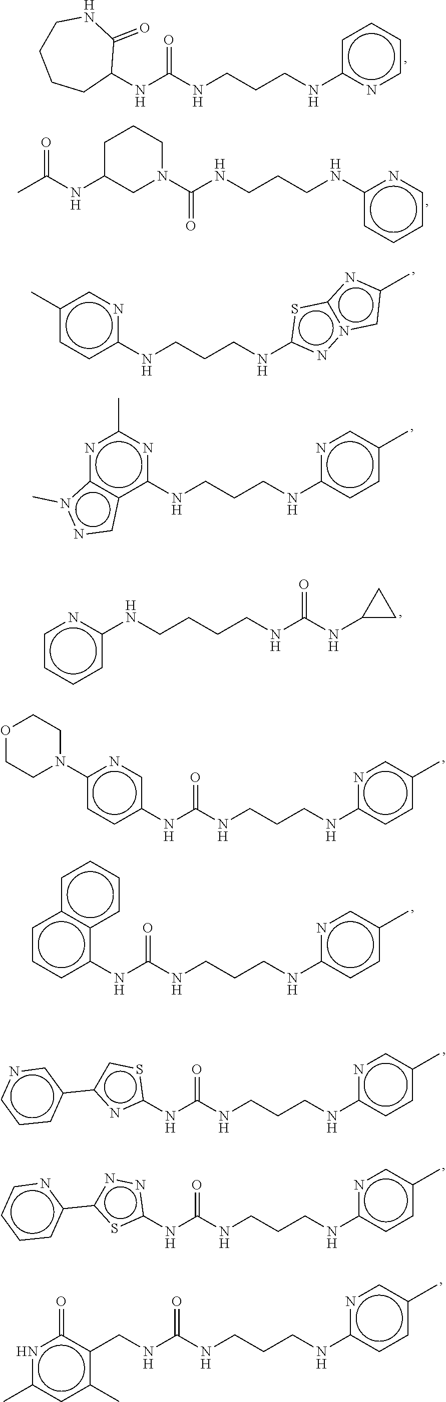

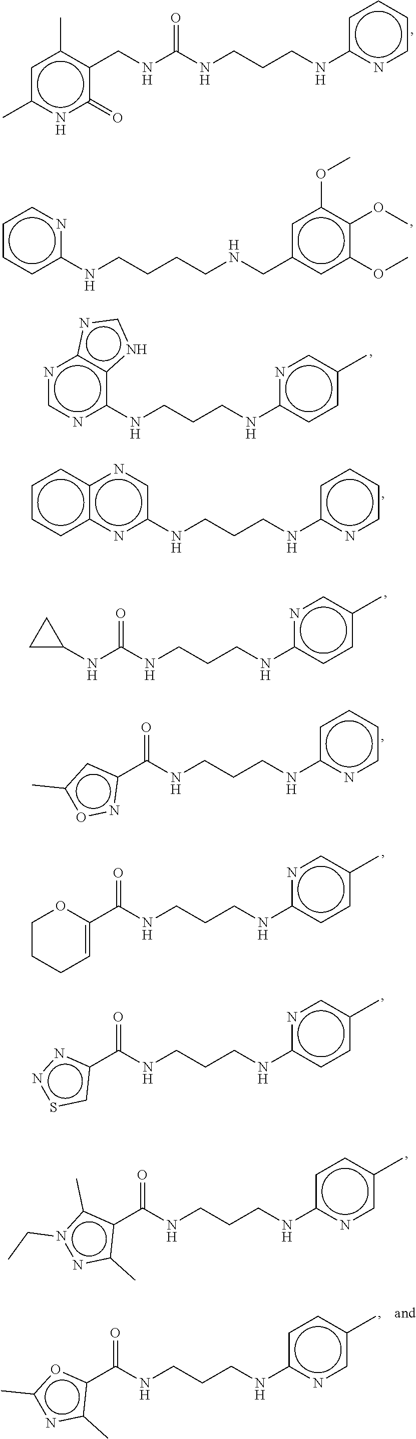

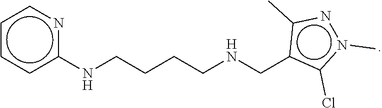

In another aspect, the present disclosure provides a compound of Formula (II):

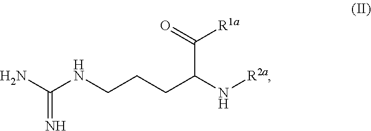

wherein
R^{1a} is selected from the group consisting of —OH, —OC₁₋₆ alkyl, and —NHR^{3a};
R^{2a} is selected from the group consisting of —H, —C(O)—R^{4a}, —C(Y)C₁₋₆ alkyl, —C(Y)C₃₋₈ cycloalkyl, —C(O)OC₁₋₆ alkyl, —C(Y)aryl, —C(Y)NH-aryl, —C(Y)NH—CH₂-aryl, —C(Y)NHC₃₋₈ cycloalkyl, and —S(O)₂aryl, wherein —C(O)—R^{4a}, —C(Y)C₁₋₆ alkyl, —C(Y)C₃₋₈ cycloalkyl, —C(O)OC₁₋₆ alkyl, —C(Y)aryl, —C(Y)NH-aryl, —C(Y)NH—CH₂-aryl, —C(Y)NHC₃₋₈ cycloalkyl, and —S(O)₂aryl can be optionally substituted from 1 to 3 times with a substituent selected independently from C₁₋₆ alkyl and —NH₂,
R^{3a} is selected from C₁₋₆ alkyl or monocyclic or bicyclic aryl, wherein C₁₋₆ alkyl and monocyclic and bicyclic aryl can be optionally substituted from 1 to 3 times with a substituent selected independently from —COOH and —NO₂;
R^{4a} is non-aromatic heterocyclyl optionally substituted with —C(O)OC₁₋₆ alkyl;
Y is O or S,
or a solvate, or a pharmaceutically acceptable salt thereof.

In some embodiments, R^{1a} is selected from the group consisting of —OH, —OMe, —OEt,

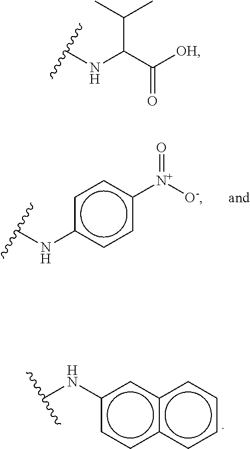

In some embodiments, R^{2a} is selected from the group consisting of —H,

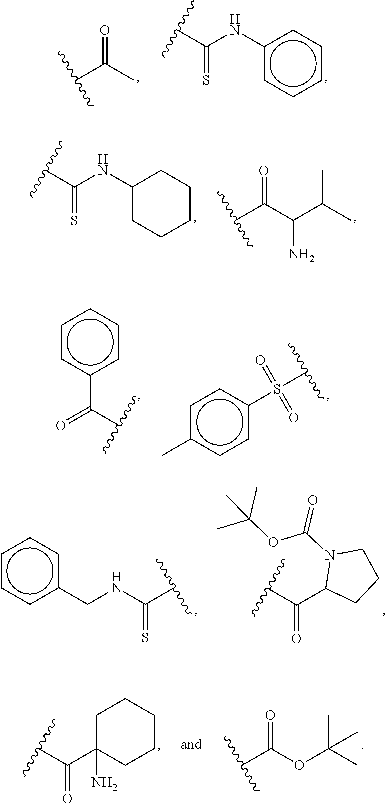

In some embodiments, the compound of Formula (II) is selected from a group consisting of

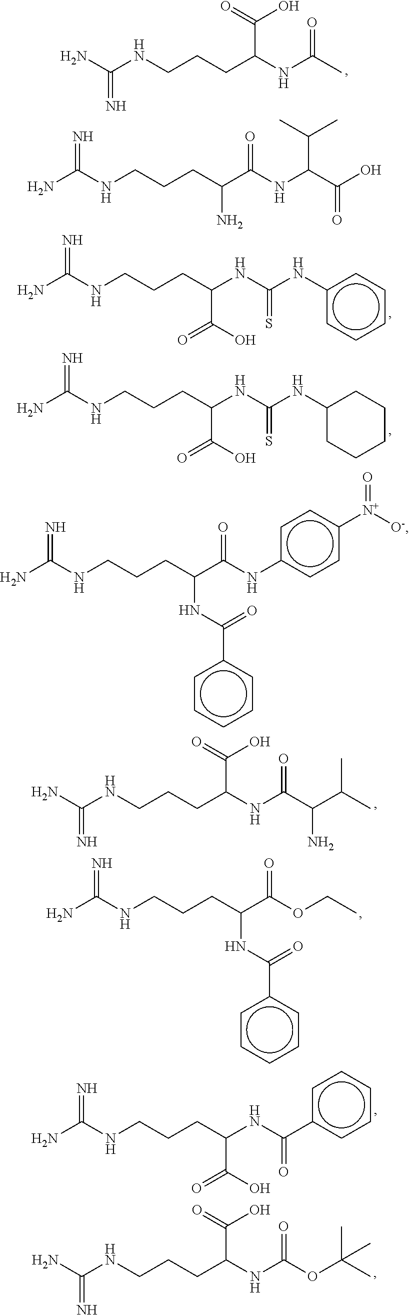

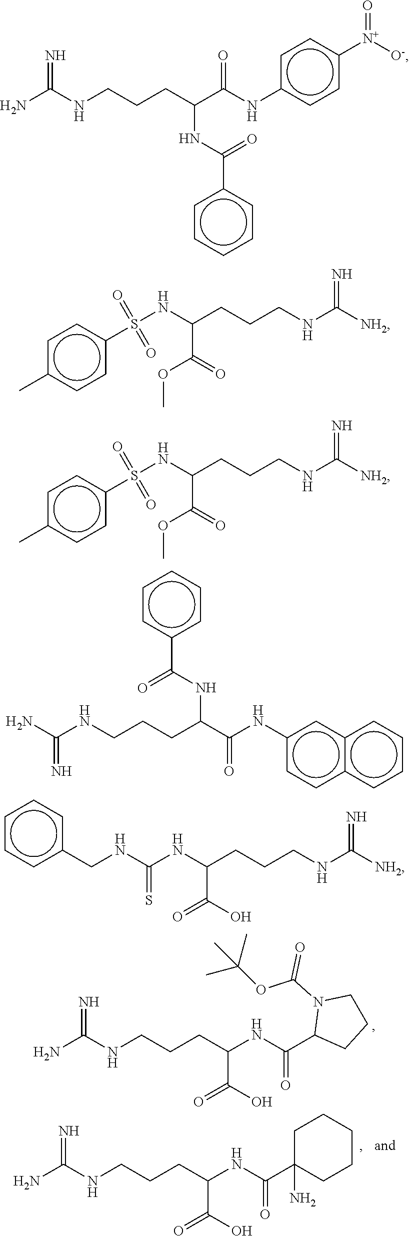

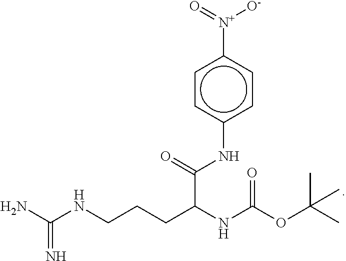

In another aspect, the present disclosure provides a compound of Formula (III):

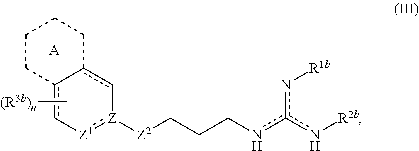

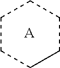

is optional, and if present is

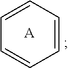

[symbol]

is a single or a double bond;
Z is C or N;
Z¹ is CH, CH₂, N, or a covalent bond;
Z² is CH₂ or a covalent bond;
n is 0, 1, or 2;
R^{1b} is selected from the group consisting of —H, C₁₋₆ alkyl, C₃₋₈ cycloalkyl, aryl, and benzyl, wherein C₁₋₆ alkyl, aryl, and benzyl can be optionally substituted with a substituent selected from —CN, —OC₁₋₆ alkyl, heteroaryl, and non-aromatic heterocyclyl;
R^{2b} is selected from the group consisting of —H, C₁₋₆ alkyl, C₃₋₈ cycloalkyl, aryl, and benzyl, wherein C₁₋₆ alkyl, aryl, and benzyl can be optionally substituted with a substituent selected from —CN, —OC₁₋₆ alkyl, heteroaryl, and non-aromatic heterocyclyl;
R^{3b} is —H or C₁₋₆ alkyl,
or a solvate, or a pharmaceutically acceptable salt thereof.

In some embodiments, R^{1b} is selected from the group consisting of —H, —Me, -Et,

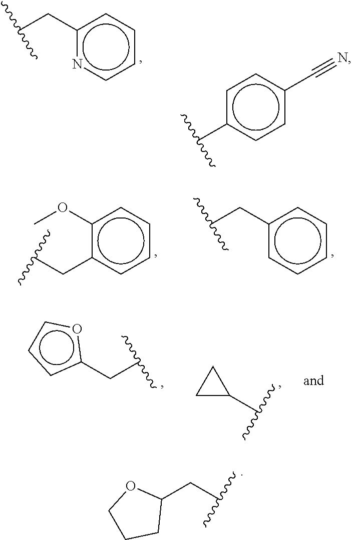

In some embodiments, the compound of Formula (III) has a Formula (IIIA):

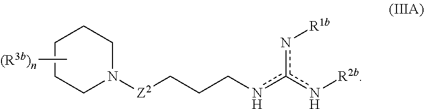

In some embodiments, the compound of Formula (III) has a Formula (IIIB):

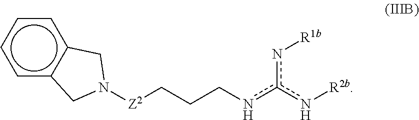

In some embodiments, the compound of Formula (III) has a Formula (IIIC):

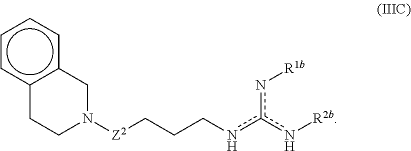

In some embodiments, the compound of Formula (III) has a Formula (IIID):

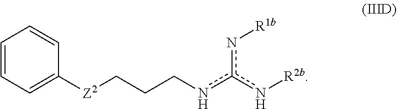

In some embodiments, the compound of Formula (III) has a Formula (IIIE):

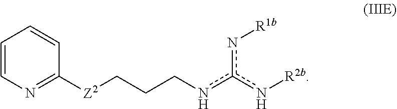

In some embodiments, the compound of Formula (III) is selected from the group consisting of

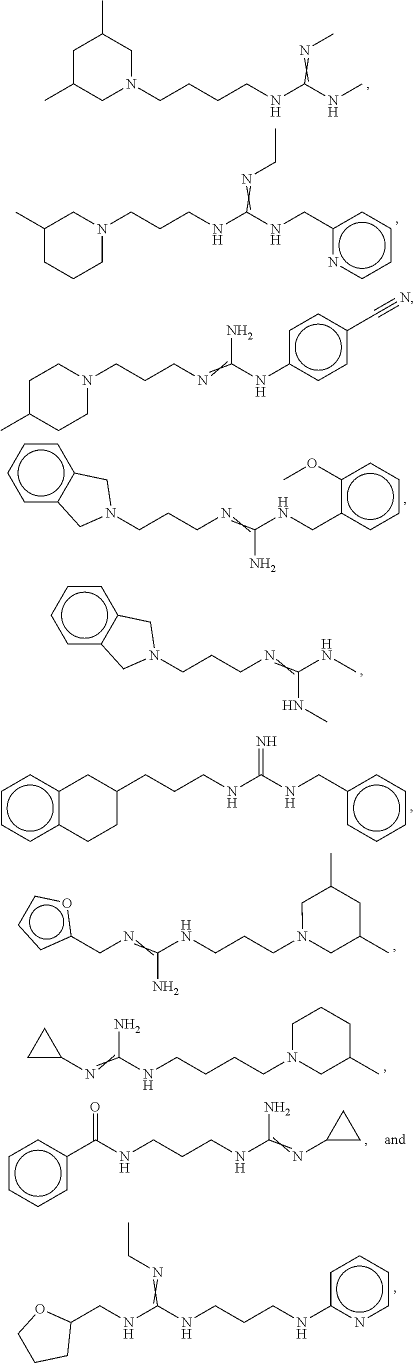

wherein [symbol] indicates that the compound is present as a cis-isomer, a trans-isomer, or a combination of cis- and trans-isomers.

According to the present disclosure, when a compound described herein contain olefinic double bonds, and unless specified otherwise, it is intended that the compounds include both a cis-isomer, a trans-isomer, or a combination of cis- and trans-isomers. In one embodiment, the compound is a cis-isomer. In another embodiment, the compound is a trans-isomer. In yet another embodiment, the compound is a mixture of cis- and trans-isomers.

In another aspect, the present disclosure provides a compound of Formula (IV):

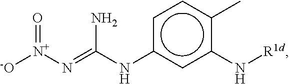

wherein
R^{1d} is —COOC₁₋₆ alkyl, —C(O)aryl, or —C(O)heteroaryl, wherein —C(O)aryl can be optionally substituted with —NO₂,
or a solvate, or a pharmaceutically acceptable salt thereof.

In some embodiments, R^{1d} is —COOMe,

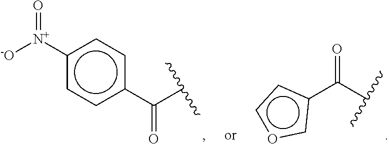

In some embodiments, the compound of Formula (IV) is selected from a group consisting of

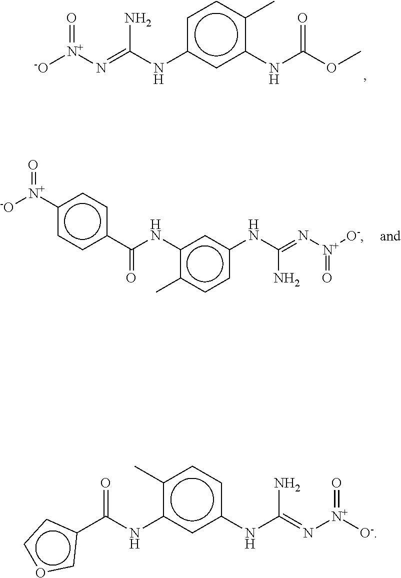

In another aspect, the present disclosure provides a compound selected from the group consisting of

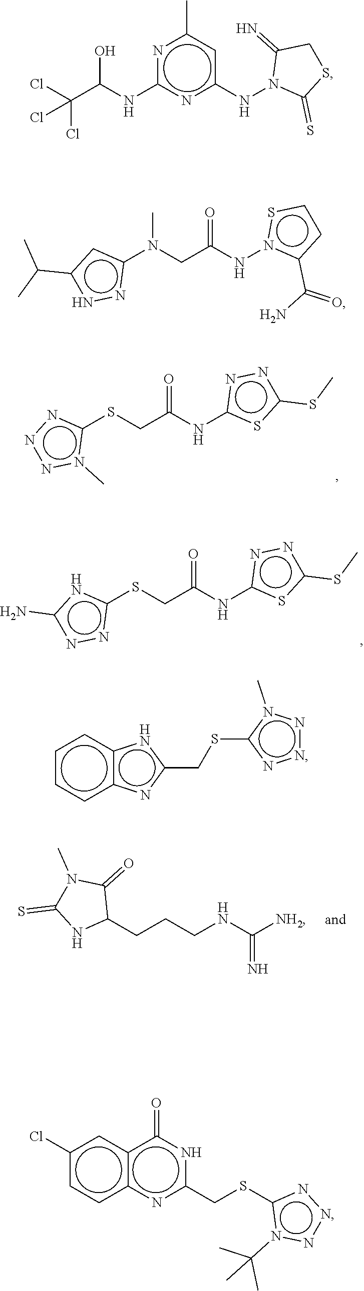

or a solvate, or a pharmaceutically acceptable salt thereof.

In another aspect, the present disclosure provides a method of identifying one or more compounds capable of regulating Cav2.2. This method comprises providing a CBD3 peptide and identifying one or more stable structural motif(s) of the CBD3 peptide. This method further comprises providing one or more pharmacophore models that map the stable structural motif of the CBD3 peptide or it's properties into said pharmacophore models. This method further comprises providing one or more compounds and screening each of the pharmacophore models against these compounds. This method further comprises selecting one or more compounds that have the highest Cav2.2 recognition score for future testing.

In one embodiment, the stable structural motifs of the CBD3 peptide are identified using molecular dynamics trajectories. Molecular dynamics of the active peptide are performed in triplicate over 500 ns each. Trajectories are clustered at each amino acid to identify stable structural motifs that can act as anchors of the interaction between the CBD3 peptide and Cav2.2.

In one embodiment, at least one stable structural motif of the CBD3 peptide is identified.

In another embodiment, at least two stable structural motifs of the CBD3 peptide are identified.

In another embodiment, at least three stable structural motifs of the CBD3 peptide are identified.

In yet another embodiment, at least four stable structural motifs of the CBD3 peptide are identified.

In some embodiments, the stable structural motif comprises a basic group and a hydrophobic moiety.

In some embodiments, the stable structural motifs are Arg and Ala.

Once the stable structural motifs are identified, the three dimensional (3D) chemotypes (i.e., chemical properties) of these stable structural motifs are mapped into different pharmacophore models to cover all possible meaningful protein-protein interactions that the stable structural motifs of CBD3 could have with a generic binding site in Cav2.2.

In some embodiments, the pharmacophore models are three dimensional (3D) models. Suitable 3D models that can be used include, but are not limited to, hydrogen bonds donor and acceptors and hydrophobic groups.

Each 3D pharmacophore model is than screened against a set of commercially available compounds. Compounds that optimally sampled the designed pharmacophores are selected for future testing.

In some embodiments, compounds that have the highest similarity score to the selected stable structural motifs are selected for future testing.

In one embodiment, 27 million commercially available compounds in the server ZincPharmer were used for screening and a total of 77 compounds were selected for future testing.

### Therapeutic Formulations, Administration and Uses

The present disclosure provides pharmaceutical compositions comprising the compound of the present disclosure.

The compositions of the disclosure may be formulated with suitable carriers, excipients, and other agents that provide improved transfer, delivery, tolerance, and the like. A multitude of appropriate formulations may be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of a compound administered to a patient may vary depending upon the age and the size of the patient, target disease, conditions, route of administration, and the like. The suitable dose is typically calculated according to body weight or body surface area. When a compound of the present disclosure is used for therapeutic purposes in an adult patient, it may be advantageous to intravenously administer the compound of the present disclosure normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment may be adjusted. Effective dosages and schedules for administering a compound may be determined empirically; for example, patient progress may be monitored by periodic assessment, and the dose adjusted accordingly. Moreover, interspecies scaling of dosages may be performed using well-known methods in the art (e.g., Mordenti et al., 1991, Pharmaceut. Res. 8:1351).

Various delivery systems are known and may be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration may be systemic or local.

A pharmaceutical composition of the present disclosure may be delivered subcutaneously or intravenously with a standard needle and syringe. In addition, with respect to subcutaneous delivery, a pen delivery device can be used to deliver a pharmaceutical composition of the present disclosure. Such a pen delivery device may be reusable or disposable. A reusable pen delivery device generally utilizes a replaceable cartridge that contains a pharmaceutical composition. Once all of the pharmaceutical composition within the cartridge has been administered and the cartridge is empty, the empty cartridge may readily be discarded and replaced with a new cartridge that contains the pharmaceutical composition. The pen delivery device may then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition may be delivered in a controlled release system. In one embodiment, a pump may be used (see Langer, supra; Sefton, 1987, CRC Crit. Ref Biomed. Eng. 14:201). In another embodiment, polymeric materials may be used; see, Medical Applications of Controlled Release, Langer and Wise (eds.), 1974, CRC Pres., Boca Raton, Florida. In yet another embodiment, a controlled release system may be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the compound or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Therapeutic Uses of the Compounds

In another aspect, the compounds disclosed herein are useful, inter alia, for the treatment, prevention and/or amelioration of a disease, disorder or condition in need of such treatment.

In another aspect, the present disclosure provides a method of regulating Cav2.2. This method comprises contacting Cav2.2 with the compound according to the disclosure, the composition comprising any compound according to the present disclosure, or the dosage form comprising any compound according to the present disclosure.

In some embodiments, the compounds disclosed herein are useful for inhibiting Cav2.2.

In some embodiments, the compounds disclosed herein are useful for reducing membrane expression of Cav2.2.

In some embodiments, the compounds disclosed herein are useful for is uncoupling the Cav2.2-CRMP2 interaction.

In another aspect, the present disclosure provides a method of treating, reducing or alleviating pain in an individual in need of such treatment. This method comprises administering to the individual an effective amount of the compound according to the disclosure, the composition comprising any compound according to the present disclosure, or the dosage form comprising any compound according to the present disclosure.

The pain to be treated may be selected from the group consisting of acute pain, chronic pain (e.g., peripheral neuropathic pain, central neuropathic pain, or musculoskeletal pain), visceral pain, headache pain, inflammatory pain, and mixed pain.

In some embodiments, the pain being treated is selected from the group consisting of nociceptive pain, post-operative (surgical) pain, postherpetic neuralgia, traumatic nerve injury, nerve compression or entrapment, small fibre neuropathy, diabetic neuropathy, neuropathic cancer pain, failed back surgery Syndrome, trigeminal neuralgia, phantom limb pain, neuroma pain, complex regional pain syndrome, chronic arthritic pain and related neuralgias, osteoarthritic pain, fibromyalgia syndrome, interstitial cystitis, irritable bowel syndrome, Crohn's disease, chronic pelvic pain syndrome, spinal cord injury, rheumatoid arthritis, endometriosis, post-herpetic neuralgias, migraine, cluster headache, tension headache syndrome, facial pain, headache caused by other diseases, lower back pain, neck and shoulder pain, burning mouth syndrome, complex regional pain syndrome, multiple sclerosis related pain, Parkinson disease related pain, post-stroke pain, post-traumatic spinal cord injury pain, pain in dementia, pain associated with diabetes, pain associated with cancer, chemotherapy, and HIV or HIV treatment-induced neuropathy.

According to the present disclosure, if osteoarthritic pain is treated, joint mobility can also improve as the underlying chronic pain is reduced. Thus, at least one compound for treatment of osteoarthritic pain inherently will also improve joint mobility in patients suffering from osteoarthritis.

In some embodiments, the pain to be treated selected from the group consisting of is neuropathic pain, pain associated with trauma, pain associated with therapy, inflammatory pain, and pain secondary to cancer.

In some embodiments, the individual being treated has been diagnosed with or is at risk for developing pain related to pathological inflammation.

In some embodiments, the pain is caused by diabetic neuropathy or similar conditions.

In some individuals, the pain may be the result of treatment with other compounds to treat conditions such as cancer, HIV-AIDS and the like.

In some embodiments, the pain being treated is due to stroke (the neuronal damage resulting from head trauma), mood disorders, epilepsy, schizophrenia, or neurodegenerative disorders.

In some embodiments, the compound according to the disclosure can be used in combination therapy. For example, the compound according to the disclosure can be used in combination with another anti-pain agent. Suitable anti-pain agents that can be used include, but are not limited to, steroids, NSAIDS (COX-2 inhibitors, salicylates, indoleacetic acid derivatives, fenamates, benzothiazine derivatives, pyrrolacetic acids), and analgesics and opiods (lidocaine, morphine, fentanyl, midazolam, propofol, lorazepam, haloperidol, thiopental, pentobarbital, diazepam).

### EXAMPLES

The following examples illustrate specific aspects of the instant description. The examples should not be construed as limiting, as the examples merely provide specific understanding and practice of the embodiments and their various aspects.

### Abbreviations

Caᵥ2.2, N-type voltage-gated calcium channel; Caᵥ1, L-type voltage-gated calcium channel; Caᵥ2.1, P/Q-type voltage-gated calcium channel; Caᵥ2.3, R-type voltage-gated calcium channel; Caᵥ3, T-type voltage-gated calcium channel; CBD3, calcium channel binding domain 3; CRMP2, collapsin response mediator protein 2; DRG, dorsal root ganglia; i.t. intrathecal; MDS, molecular dynamics simulations; HVA, high voltage activated; Naᵥ1.7, voltage-gated sodium channel isoform 7.

### Nomenclature of Targets and Ligands

Key protein targets and ligands in this disclosure are hyperlinked to corresponding entries in http://www.guidetopharmacology.org, and are permanently archived in the Concise Guide to PHARMACOLOGY 2021/22 (Alexander et al., 2021).

### Example 1: Rational Design of Pharmacophore Models in the Absence of Receptor Structure Molecular Dynamics of CBD3 Peptide

The CBD3 peptide was modeled based on the X-ray diffraction structure of CRMP2 (PDB: 5MKV) (Zheng et al., 2018). Three independent molecular dynamics simulations (MDS) of both the CBD3 by itself and conjugated with a blood-brain barrier-permeable peptide TAT-CBD3 were run with pmemd.cuda (Case et al., 2018; Götz et al., 2012; Salomon-Ferrer et al., 2013) from AMBER18 using AMBER ff14SB force field (Maier et al., 2015). tLeap binary (AMBER18) was used for solvating the peptides in an octahedral TIP3P water box with a 15 Å distance from structure surface to the box edges, and closeness parameter of 0.75 Å. The neutralized system was solvated in a solution of 150 mM NaCl. H-bonds were constrained using SHAKE algorithm and integration time-step at 2 fs. Simulations were carried out equilibrating the system for 1 ns at NPT using Berstein barostat to keep constant pressure at 1 atm at 300K, followed by 300 ns NPT production at 300 K. The first 60 ns of each MDS were discarded as equilibration time.

### Anchor Prediction

Hierarchical clustering (Kozakov et al., 2005) determined the most stable conformation of dipeptides between A₁ and L₅. Clustering was based on the Root Mean Square Deviation (RMSD) between MDS snapshots less than 1 Å for A₁R², R₂S₃, S₃R₄, and R₄L₅. The contacts of side chains were also determined as a proxy for ability to bind the receptor, i.e., if side chains were interacting with each other, their interaction with the receptor was hindered. Atomic contacts were defined as atoms from the peptide that were less than 3.8 Å of Cβ-alanine, [Nε, NH2]-arginine, Cβ-serine, [Cδ1, Cδ2]-leucine from dipeptides. The prediction was that the stable motif accessible to solvent was critical for molecular recognition, i.e., the anchor of the protein-protein interaction (Rajamani et al., 2004).

### Virtual Screening of ZINC Database

The anchor motif was used as a template to design and refine pharmacophore models to virtually screen more than 27 million compounds using the public server ZINCPharmer (Koes & Camacho, 2012). Based on A₁R₂ configuration near to 27 million commercially available compounds were screened using ZINCPharmer, resulting in the compounds described in the present disclosure.

### Example 2: Synthesis of CBD3063

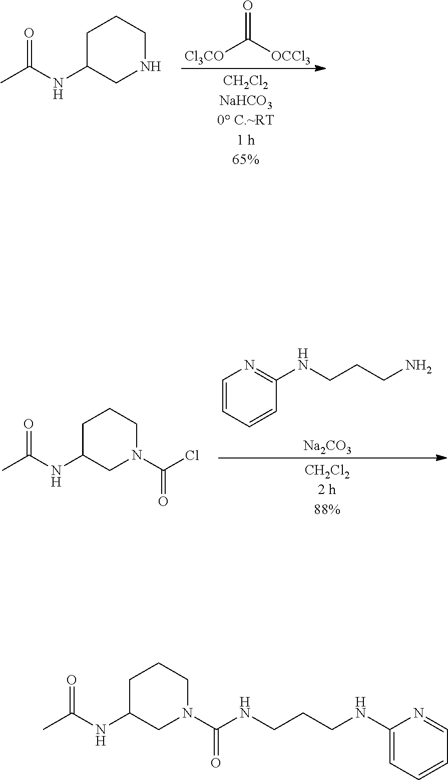

### Step 1. Synthesis of 3-Acetamidopiperidine-1-carbonyl Chloride

In a cooled (0° C.) solution of N-(piperidin-3-yl)acetamide (500 mg, 3.52 mmol) in anhydrous dichloromethane (20 mL) was added NaHCO₃ (1.10 g, 10.6 mmol) and triphosgene (696 mg, 2.34 mmol). The mixture was stirred at room temperature for 1 hour. After all starting material has been consumed, the mixture was filtered, and the collected filtrate was evaporated under reduced pressure. The resulting residue was then allowed to pass through a short silica plug (wash with 100% EtOAc) to yield crude 3-acetamidopiperidine-1-carbonyl chloride (467 mg, 65%) as sticky transparent liquid (HRMS calcd for C₈H₁₄ClN₂O₂⁺ [M+H]⁺: 205.0748; found: 205.0738). The compound was used immediately for the next step without further purification.

### Step 2. Synthesis of 3-Acetamido-N-(3-(pyridin-2-ylamino)propyl)piperidine-1-carboxamide

3-Acetamidopiperidine-1-carbonyl chloride (100 mg, 0.489 mmol) from the previous step was dissolved in anhydrous dichloromethane (5.0 mL). Into this solution was added Na₂CO₃ (104 mg, 0.977 mmol) and N¹-(pyridin-2-yl)propane-1,3-diamine (73.9 mg, 0.489 mmol). The mixture was stirred for 2 hours, upon which all starting material had reacted. The mixture was filtered, and the collected filtrate was evaporated under reduced pressure. The resulting residue was then purified by flash column chromatography (gradient elution of 0% to 10% MeOH in CH₂Cl₂) to yield 3-acetamido-N-(3-(pyridin-2-ylamino)propyl)piperidine-1-carboxamide (137 mg, 88%) as white foam solid.

¹H NMR (600 MHz, CDCl₃) δ 7.99 (dd, J=4.1, 0.8 Hz, 1H), 7.38 (ddd, J=8.7, 7.1, 1.9 Hz, 1H), 6.53 (ddd, J=7.0, 5.2, 0.8 Hz, 1H), 6.41 (d, J=8.4 Hz, 1H), 6.12 (d, J=6.5 Hz, 1H), 5.79 (t, J=5.5 Hz, 1H), 5.10-4.95 (m, 1H), 3.99-3.86 (m, 1H), 3.45-3.36 (m, 6H), 3.32 (q, J=6.1 Hz, 2H), 1.95 (s, 3H), 1.87-1.62 (m, 5H), 1.61-1.49 (m, 1H). ¹³C NMR (151 MHz, CDCl₃) δ 170.10, 158.60, 158.46, 147.02, 137.59, 112.50, 108.28, 48.66, 45.61, 44.60, 38.63, 37.69, 30.22, 29.40, 23.37, 22.23. HRMS caled for C₁₆H₂₅N₅O₂Na [M+Na]⁺: 342.1900; found: 342.1908.

### Example 3: Culturing of CAD Cell Lines

Mouse neuron derived Cathecholamine A differentiated CAD cells (ECACC Cat #08100805, RRID: CVCL_0199) were grown in standard cell culture conditions, 37° C. in 5% (vol/vol) CO₂. The cells were maintained in DMEM/F12 media supplemented with 10% (vol/vol) FBS (HyClone) and 1% penicillin/streptomycin sulfate from 10,000 μg/mL stock.

### Example 4: Immunoprecipitation (IP) of Endogenous CRMP2

CAD cells were incubated overnight with a vehicle (0.1% DMSO) or CBD3063 (20 μM). The next day the cells were lysed into the IP buffer containing 20 mM Tris-HCl pH=7.4, 50 mM NaCl, 2 mM MgCl₂, 10 mM N-Ethylmaleimide (NEM), 1% (vol/vol) NP-40, 0.5% (mass/vol) sodium deoxycholate, 0.1% (mass/vol) sodium dodecyl sulfate (SDS) with protease inhibitors (Cat #B14002, Biotool, Houston, TX), phosphatase inhibitors (Cat #B15002, Biotool, Houston, TX) and Nuclease (Cat #88701, Thermo Fisher Scientific, Waltham, MA). Total protein concentration was determined by BCA protein assay kit (Cat #PI23225, Thermo Fisher Scientific, Waltham, MA). Five hundred micrograms of total protein were incubated overnight with 2 μg of CRMP2 antibody (Cat #C2993, Sigma-Aldrich, St. Louis, MO) at 4° C. under gentle agitation. Protein G magnetic beads (Cat #10004D, Thermo Fisher Scientific, Waltham, MA), pre-equilibrated with the immunoprecipitation buffer, were then added to the lysates and incubated for 2 hours at 4° C. to capture immuno-complexes. Beads were washed four times with IP buffer to remove nonspecific binding of proteins, before resuspension in Laemmli buffer and boiling at 95° C. for 5 min prior to immunoblotting.

### Example 5: Immunoblot Preparation and Analysis

Indicated samples were loaded on 4-20% Novex gels (cat. no. XP04205BOX; Thermo Fisher Scientific, Waltham, MA). Proteins were transferred to preactivated PVDF membranes for 1 hour at 100 V using TGS [25 mM Tris, pH 8.5, 192 mM glycine, 0.1% (mass/vol) SDS], 20% (vol/vol) methanol as transfer buffer (0.45 μm; Cat #IPVH00010; Millipore Sigma, St. Louis, MO). After transfer, the membranes were blocked at room temperature for 1 hour with TBST (50 mM Tris·HCl, pH 7.4, 150 mM NaCl, 0.1% Tween 20) with 5% (mass/vol) nonfat dry milk, and then incubated overnight at 4° C. separately with indicated primary antibodies, β111-Tubulin (Cat #G7121; Promega, Madison, WI), CRMP2 (Cat #C2993; Sigma-Aldrich, St. Louis, MO), Caᵥ2.2 (Cat #TA308673; Origene, Rockville, MD), CRMP2 pSer522 (Cat #CP2191; ECM Biosciences, Versailles, KY), and CRMP2 pThr514 (Cat #PA5-110113; Invitrogen, Waltham, MA), in TBST, 5% (mass/vol) BSA. For examining the effect of CBD3063 on CRMP2 phosphorylation state, CAD cells were treated overnight with vehicle (0.1% DMSO) or CBD3063 (20 μM) and the next day cells were lysed using RIPA buffer. Approximately 40 μg of total proteins were loaded on an SDS-PAGE and then transferred to polyvinylidene difluoride membranes and blocked at room temperature for 1 hour. Primary antibodies used for probing were CRMP2 (Cat #C2993, Sigma-Aldrich, St Louis, MO), CRMP2 pThr514 (Cat #PB-043, Kinasource, Dundee, Scotland, United Kingdom), CRMP2 pSer522 (Cat #CP2191, ECM Biosciences, Versailles, KY), and CRMP2 pT555 (Cat #CP2251, ECM Biosciences, Versailles, KY). Following incubation in HRP-conjugated secondary antibodies from Jackson Immuno Research (West Grove, PA), blots were revealed by enhanced luminescence (WBKLS0500; Millipore Sigma St. Louis, MO).

### Example 6: Animals

Pathogen-free adult male and female Sprague-Dawley rats (˜75-100 g, Charles River Laboratories, Wilmington, MA.) were kept in light (12-h light: 12-h dark cycle; lights on at 07:00 h) and temperature (23±3° C.) controlled rooms. Standard rodent chow and water were available ad libitum.

### Example 7: Dorsal Root Ganglion Neuron Cultures

Lumbar DRGs were dissected from 100 g female Sprague-Dawley rats using procedures as described previously (Gomez et al., 2022). DRGs were excised and placed in sterile DMEM (Cat #11965; Thermo Fisher Scientific, Waltham, MA). The ganglia were dissociated enzymatically with collagenase type I (5 mg/mL, Cat #LS004194; Worthington) and neutral protease (3.125 mg/mL, Cat #LS02104; Worthington, Lakewood, NJ) for 50 minutes at 37° C. under gentle agitation. The dissociated cells were then centrifuged (800 rpm for 5 min) and resuspended in DMEM containing 1% penicillin/streptomycin sulfate (Cat #15140, Life Technologies, Carlsbad, CA), 10% fetal bovine serum [HyClone]), and 30 ng/mL nerve growth factor (Cat #N2513, Millipore Sigma, St. Louis, MO). The cells were seeded on poly-D-lysine (Cat #P6407, Millipore Sigma, St. Louis, MO) and laminin (Cat #sc-29012, Santa Cruz Biotechnology, Dallas, TX) -coated 12- or 15-mm glass coverslips and incubated at 37° C. All cultures were used within 48 hours.

### Example 8: Immunocytochemistry and Confocal Microscopy

Immunocytochemistry was performed on DRG neurons incubated with vehicle (0.1% DMSO) or CBD3063 (20 μM) overnight. Cultured DRG neurons were fixed using ice-cold methanol for 5 min and then allowed to dry at room temperature. Fixed cells were rehydrated in PSB and then blocked with PBS containing 3% bovine serum albumin for 30 min at room temperature. Cell staining was performed with anti-Caᵥ2.2 (Origene, Cat #TA308673, Rockville, MD) in PBS with 3% BSA overnight at 4° C. The cells were then washed thrice in PBS and incubated with PBS containing 3% BSA and secondary antibodies (Alexa 488 Chicken anti-Rabbit (Life Technologies, Carlsbad, CA)) for 1 hour at room temperature. Coverslips were mounted and stored at 4° C. until analysis. Immunofluorescent micrographs were acquired on a Leica SP8 inverted upright microscope using a 63X, oil immersion objective. For all quantitative comparisons among cells under differing experimental conditions, camera gain and other relevant settings were kept constant. The freeware image analysis program Image J (http://rsb.info.nih.gov/ij/) was used for quantifying cellular fluorescence. Regions of interest (i.e. cells) were defined by hand using Image J.

### Example 9: Calcium Imaging

Changes in depolarization-induced calcium influx in rat DRG neurons were determined by loading neurons with 3 mM Fura-2AM for 30 minutes at 37° C. (Cat #F1221; Thermo Fisher Scientific, Waltham, MA, stock solution prepared at 1 mM in DMSO, 0.02% pluronic acid, Cat #P-3000MP; Life Technologies, Carlsbad, CA) as previously described (Bellampalli et al., 2019). DRG neurons were incubated overnight with 20 μM of test compounds. A standard bath solution containing 139 mM NaCl, 3 mM KCl, 0.8 mM MgCl₂, 1.8 mM CaCl₂, 10 mM Na-HEPES, 5 mM glucose, pH 7.4, was used. Depolarization was evoked with a 10 sec pulse of 90 mM KCl. Fluorescence imaging was achieved with an inverted microscope, Nikon Eclipse TE2000-U, using an objective Nikon Super Fluor 4X and a Photometrics-cooled CCD camera CoolSNAPHQ (Roper Scientific, Tucson, AZ) controlled by Nis Elements software (version 4.20; Nikon Instruments, Melville, NY). The excitation light was delivered by a Lambda-LS system (Sutter Instruments, Novato, CA). The excitation filters (340±5 nm and 380±7 nm) were controlled by a Lambda 10 to 2 optical filter change (Sutter Instruments, Novato, CA). Fluorescence was recorded through a 505-nm dichroic mirror at 535±25 nm. Images were taken every ˜2.4 seconds during the time course of the experiment to minimize photobleaching and phototoxicity. To provide acceptable image quality, a minimal exposure time that provided acceptable image quality was used. Changes in [Ca²⁺]c were monitored following a ratio of F₃₄₀/F₃₈₀, calculated after subtracting the background from both channels.

### Example 10: Whole-Cell Patch-Clamp Recordings of Ca²⁺ and Na⁺ Currents in Acutely Dissociated DRG Neurons

Recordings were obtained from acutely dissociated DRG neurons as described earlier (Bellampalli et al., 2019). Patch-clamp recordings were performed at room temperature (22-24° C.). Currents were recorded using an EPC 10 Amplifier-HEKA (HEKA Elektronik, Ludwigshafen, Germany) linked to a computer with Patchmaster software. DRG neurons were incubated overnight (˜16-24 hours) with 20 μM of CBD3063.

For total calcium current (ICa²⁺) recordings, the external solution consisted of the following (in mM): 110 N-methyl-D-glucamine, 10 BaCl₂, 30 TEA-Cl, 10 HEPES, 10 glucose, and 0.001 TTX (pH 7.29 adjusted with TEA-OH, and mOsm/L=310). Patch pipettes were filled with an internal solution containing (in mM): 150 CsCl₂, 10 HEPES, 5 Mg-ATP, and 5 BAPTA, (pH 7.2 adjusted with CsOH, and mOsm/L-305). Peak Ca²⁺ current was acquired by applying 200-millisecond voltage steps from −70 to +60 mV in 10-mV increments from a holding potential of −90 mV to obtain the current-voltage (I-V) relation. To measure the different subtypes of Ca²⁺ channels, DRGs were treated with a Caᵥ inhibitor cocktail omitting the inhibitor specific to the subtype being tested (e.g., to measure Caᵥ2.2 currents, ω-conotoxin GVIA is omitted): Nifedipine (10 μM, L-type), ω-Conotoxin-GVIA (500 nM, P/Q-type) (Feng, et al., 2001), SNX482 (200 nM, R-type) (Newcomb et al., 1998), ω-agatoxin (200 nM, P/Q-type) (Mintz et al., 1992), TTA-P2 (1 μM, T-type) (Choe et al., 2011).

For Na⁺ current (I_{Na}⁺) recordings, the external solution contained (in mM): 130 NaCl, 3 KCl, 30 tetraethylammonium chloride, 1 CaCl₂, 0.5 CdCl₂, 1 MgCl₂, 10 D-glucose, and 10 HEPES (pH 7.3 adjusted with NaOH, and mOsm/L=324). Patch pipettes were filled with an internal solution containing (in mM): 140 CsF, 1.1Cs-EGTA, 10 NaCl, and 15 HEPES (pH 7.3 adjusted with CsOH, and mOsm/L=311). Peak Na⁺ current was acquired by applying 150-millisecond voltage steps from −70 to +60 mV in 5-mV increments from a holding potential of −60 mV to obtain the current-voltage (I-V) relation.

To isolate potassium currents (I_{K+}), DRG neurons were bathed in external solution composed of (in millimolar): 140 N-methyl-glucamine chloride, 5 KCl, 1 MgCl₂, 2 CaCl₂, 10 D-glucose, and 10 HEPES (pH adjusted to 7.3 with KOH and mOsm/L=313). Recording pipettes were filled with internal solution containing (in mM): 140 KCl, 2.5 MgCl₂, 4 Mg-ATP, 0.3 Na-GTP, 2.5 CaCl2), 5 EGTA, and 10 HEPES (pH adjusted to 7.3 with KOH and mOsm/L=320). From a holding potential of −60 mV, IK activation was determined by applying 300-millisecond voltage steps from −80 to +60 mV in 10-mV increments.

Normalization of currents to each cell's capacitance (pF) was performed to allow for collection of current density data. For I-V curves, functions were fitted to data using a non-linear least squares analysis. I-V curves were fitted using double Boltzmann functions:

$$f = a + g 1 / ( 1 + exp ( ( x - {V}_{1 / 2} 1 ) / k 1 ) ) + g 2 / ( 1 + exp ( - ( x - {V}_{1 / 2} 2 ) / k 2 ) )$$

where x is the pre-pulse potential, V_{1/2} is the mid-point potential, and k is the corresponding slope factor for single Boltzmann functions. Double Boltzmann fits were used to describe the shape of the curve, not to imply the existence of separate channel populations. Numbers 1 and 2 simply indicate first and second mid-points; a along with g are fitting parameters.

Activation curves were obtained from the I-V curves by dividing the peak current at each depolarizing step by the driving force according to the equation: G=I/(Vₘₑₘ-Eᵣₑᵥ), where I is the peak current, Vₘₑₘ is the membrane potential and Eᵣₑᵥ is the reversal potential. The conductance (G) was normalized against the maximum conductance (Gₘₐₓ). For total and the different subtypes of Ca²⁺ currents, steady-state inactivation (SSI) curves were obtained by applying an H-infinity protocol that consisted of 1.5-seconds conditioning pre-pulses from −100 to +30 mV in 10-mV increments followed by a 20-millisecond test pulse to +10 mV. For Na⁺ currents, SSI curves were obtained by applying an H-infinity protocol that consisted of 1-second conditioning pre-pulses from −120 to +10 mV in 10-mV increments followed by a 200-millisecond test pulse to +10 mV. Inactivation curves were obtained by dividing the peak current recorded at the test pulse by the maximum current (Iₘₐₓ). Activation and SSI curves were fitted with the Boltzmann equation.

### Example 11: Indwelling Intrathecal Catheter

Rats were anesthetized with ketamine/xylazine 80/12 mg/kg intraperitoneally (i.p.) (Sigma-Aldrich, St. Louis, MO), and their head was placed in a stereotaxic frame. The cisterna magna was exposed and incised. As previously reported, an 8-cm catheter (PE-10; Stoelting, Wood Dale, IL) was implanted, terminating in the lumbar region of the spinal cord (Yaksh & Rudy, 1976). Catheters were sutured (using 3-0 silk sutures) into the deep muscle and externalized at the back of the neck. Autoclips were used to close the skin, and other surgeries were performed after a 5- to 7-day recovery period.

### Example 12: Spared Nerve Injury (SNI) Model of Neuropathic Pain

The neuropathic pain model induced by spared nerve injury (SNI) in rats was performed as previously described. Adult male rats (250 g, Envigo, Placentia, CA) were anesthetized with isoflurane (5% induction, 2% maintenance in 2 L/min air), and skin on the lateral surface of the left hind thigh was incised. Then, the biceps femoris muscle was dissected to expose the three terminal branches of the sciatic nerve. The common peroneal and tibial branches were tightly ligated with 4-0 silk and axotomized 2.0 mm distal to the ligation. Closure of the incision was made in two layers. The muscle was sutured once with 5-0 absorbable suture; skin was autoclipped. Animals were allowed to recover for 7 days before any testing. On the 7^{th} day after SNI, CBD3063 (0.3 μg/kg) or 1% DMSO was injected intrathecally. Mechanical allodynia was assessed 10 days after surgery.

### Example 13: Measurement of Mechanical Allodynia

Mechanical allodynia was assessed by measuring rats' paw withdrawal threshold in response to probing with a series of fine calibrated filaments (von Frey, Stoelting, Wood Dale, IL). Rats were placed in suspended plastic cages with wire mesh floor, and each von Frey filament was applied perpendicularly to the plantar surface of the paw. The “up-and-down” method (sequential increase and decrease of the stimulus strength) was used to determine the withdrawal threshold Dixon's nonparametric method was used for data analysis, as described by Chaplan et al., 1994. Data were expressed as the mean withdrawal threshold.

### Example 14: Data Analysis

Graphing and statistical analysis was performed with GraphPad Prism (Version 9). All data sets were checked for normality using D'Agostino & Pearson test. Details of statistical tests and significance and sample sizes are reported in the description of the figures. All data plotted represent mean±SEM. For western blot experiments, statistical differences between groups were determined by unpaired t test or Mann-Whitney test. Statistical significance of confocal imaging data was evaluated by Mann-Whitney test. For electrophysiological recordings: Normalized peak currents were analyzed by Kruskal-Wallis test followed by the Dunn's post hoc test; the significance of the I-V curves was analyzed by multiple Mann-Whitney tests; peak current density as well as V_{1/2} midpoint potential and k slope factor were compared using Mann-Whitney test. For resting membrane potential and rheobase, the significance was analyzed by Mann-Whitney test; the significance of the number of evoked action potentials per step was analyzed by multiple Mann-Whitney tests. Behavioral data was analyzed by multiple Mann-Whitney tests and Mann-Whitney test for time-course experiments and area under the curve respectively. Detailed statistical analyses are presented in Table 1.

TABLE 1
Details of Statistical Analyses for all Figures

| Fig- | | Statis- | | |
|---|---|---|---|---|
| ure | | tical | | |
| pa- | | test; | Post-hoc analysis | Number of |
| nel | Assay | findings | (adjusted p-value) | subjects |
| FIG. | % Change | One-way | Dunnett post | DMSO (n = 507) |
| 2A | in | ANOVA; | hoc test | CBD3018 (n = 249) |
| | average | p < | DMSO (0.1%) vs. | CBD3026 (n = 213) |
| | response | 0.0001 | CBD3018 (20 μM): | CBD3033 (n = 203) |
| | to 90 mM | | p < 0.0001 | CBD3038 (n = 85) |
| | KCl | | DMSO (0.1%) vs. | CBD3039 (n = 443) |
| | | | CBD3026 (20 μM): | CBD3062 (n = 387) |
| | | | p < 0.0001 | CBD3063 (n = 434) |
| | | | DMSO (0.1%) vs. | CBD3065 (n = 91) |
| | | | CBD3033 (20 μM): | CBD3074 (n = 82) |
| | | | p < 0.0001 | |
| | | | DMSO (0.1%) vs. | |
| | | | CBD3038 (20 μM): | |
| | | | p < 0.0001 | |
| | | | DMSO (0.1%) vs. | |
| | | | CBD3039 (20 μM): | |
| | | | p < 0.0001 | |
| | | | DMSO (0.1%) vs. | |
| | | | CBD3062 (20 μM): | |
| | | | p < 0.0001 | |
| | | | DMSO (0.1%) vs. | |
| | | | CBD3063 (20 μM): | |
| | | | p < 0.0001 | |
| | | | DMSO (0.1%) vs. | |
| | | | CBD3065 (20 μM): | |
| | | | p < 0.0001 | |
| | | | DMSO (0.1%) vs. | |
| | | | CBD3074 (20 μM): | |
| | | | p < 0.0001 | |
| FIG. | Nor- | Kruskal- | Dunn's post hoc test | DMSO (n = 98) |
| 2C | malized | Wallis | DMSO (0.1%) vs. | CBD3018 (n = 18) |
| | peak | test; | CBD3018 (20 μM): | CBD3026 (n = 15) |
| | total | p < | p = 0.6541 | CBD3033 (n = 13) |
| | calcium | 0.0001 | DMSO (0.1%) vs. | CBD3038 (n = 18) |
| | current | | CBD3026 (20 μM): | CBD3039 (n = 17) |
| | density | | p > 0.9999 | CBD3062 (n = 17) |
| | | | DMSO (0.1%) vs. | CBD3063 (n = 17) |
| | | | CBD3033 (20 μM): | CBD3065 (n = 16) |
| | | | p = 0.1040 | CBD3074 (n = 21) |
| | | | DMSO (0.1%) vs. | |
| | | | CBD3038 (20 μM): | |
| | | | p > 0.9999 | |
| | | | DMSO (0.1%) vs. | |
| | | | CBD3039 (20 μM): | |
| | | | p > 0.9999 | |
| | | | DMSO (0.1%) vs. | |
| | | | CBD3062 (20 μM): | |
| | | | p > 0.9999 | |
| | | | DMSO (0.1%) vs. | |
| | | | CBD3063 (20 μM): | |
| | | | p = 0.0014 | |
| | | | DMSO (0.1%) vs. | |
| | | | CBD3065 (20 μM): | |
| | | | p = 0.0024 | |
| | | | DMSO (0.1%) vs. | |
| | | | CBD3074 (20 μM): | |
| | | | p = 0.0062 | |
| FIG. | Total | Multiple | −70 mV: p = 0.5232 | DMSO (n = 13) |
| 3B | calcium | Mann- | −60 mV: p = 0.8799 | CBD3063 (n = 16) |
| | current | Whitney | −50 mV: p = 0.8206 | |
| | density | tests | −40 mV: p = 0.8459 | |
| | | | −30 mV: p = 0.8123 | |
| | | | −20 mV: p = 0.8123 | |
| | | | −10 mV: p = 0.2875 | |
| | | | 0 mV: p = 0.0251 | |
| | | | 10 mV: p = 0.0014 | |
| | | | 20 mV: p = 0.0004 | |
| | | | 30 mV: p = 0.0101 | |
| | | | 40 mV: p = 0.1207 | |
| | | | 50 mV: p = 0.8459 | |
| | | | 60 mV: p = 0.7790 | |
| FIG. | Peak total | Mann- | DMSO (0.1%) vs. | DMSO (n = 13) |
| 3C | calcium | Whitney | CBD3063 (20 μM): | CBD3063 (n = 16) |
| | current | test | p = 0.0015 | |
| | density | | | |
| FIG. | Relative | Unpaired | DMSO (0.1%) vs. | DMSO (n = 3) |
| 4B | Caᵥ2.2 | t test | CBD3063 (20 μM): | CBD3063 (n = 3) |
| | binding to | | p = 0.0034 | |
| | CRMP2 | | | |
| FIG. | Caᵥ2.2 | Mann- | DMSO (0.1%) vs. | DMSO (n = 25) |
| 4D | mem- | Whitney | CBD3063 (20 μM): | CBD3063 (n = 38) |
| | brane/ | test | p < 0.0001 | |
| | cytosol | | | |
| | ratio | | | |
| FIG. | Peak | Kruskal- | Dunn's post hoc test | DMSO (n = 30) |
| 5A | N-type | Wallis | DMSO (0.1%) vs. | CBD3063 (2 μM) |
| | calcium | test; | CBD3063 (2 μM): | (n = 13) |
| | current | p = | p > 0.9999 | CBD3063 (20 |
| | density | 0.0073 | DMSO (0.1%) vs. | μM) (n = 30) |
| | concen- | | CBD3063 (20 μM): | CBD3063 (50 |
| | tration- | | p = 0.0371 | μM) (n = 15) |
| | response | | DMSO (0.1%) vs. | |
| | | | CBD3063 (50 μM): | |
| | | | p = 0.0076 | |
| FIG. | N-type | Multiple | −70 mV: p = 0.1427 | DMSO (n = 12) |
| 5C | calcium | Mann- | −60 mV: p = 0.1147 | CBD3063 (n = 17) |
| | current | Whitney | −50 mV: p = 0.1307 | |
| | density | tests | −40 mV: p = 0.2634 | |
| | | | −30 mV: p = 0.6868 | |
| | | | −20 mV: p = 0.1797 | |
| | | | −10 mV: p = 0.1171 | |
| | | | 0 mV: p = 0.0266 | |
| | | | 10 mV: p = 0.0301 | |
| | | | 20 mV: p = 0.0725 | |
| | | | 30 mV: p = 0.0973 | |
| | | | 40 mV: p = 0.0973 | |
| | | | 50 mV: p = 0.1522 | |
| | | | 60 mV: p = 0.1280 | |
| FIG. | Peak | Mann- | DMSO (0.1%) vs. | DMSO (n = 12) |
| 5D | N-type | Whitney | CBD3063 (20 μM): | CBD3063 (n = 17) |
| | calcium | test | p = 0.0301 | |
| | current | | | |
| | density | | | |
| FIG. | L-type | Multiple | −70 mV: p = 0.9820 | DMSO (n = 8) |
| 6A | calcium | Mann- | −60 mV: p > 0.9999 | CBD3063 (n = 7) |
| | current | Whitney | −50 mV: p = 0.8443 | |
| | density | tests | −40 mV: p = 0.7789 | |
| | | | −30 mV: p = 0.9551 | |
| | | | −20 mV: p = 0.8920 | |
| | | | −10 mV: p = 0.3969 | |
| | | | 0 mV: p = 0.3357 | |
| | | | 10 mV: p = 0.3357 | |
| | | | 20 mV: p = 0.2810 | |
| | | | 30 mV: p = 0.1206 | |
| | | | 40 mV: p = 0.2810 | |
| | | | 50 mV: p = 0.6943 | |
| | | | 60 mV: p = 0.1520 | |
| FIG. | Peak | Mann- | DMSO (0.1%) vs. | DMSO (n = 8) |
| 6B | L-type | Whitney | CBD3063 (20 μM): | CBD3063 (n = 7) |
| | calcium | test | p = 0.2810 | |
| | current | | | |
| | density | | | |
| FIG. | P/Q-type | Multiple | −70 mV: p = 0.7007 | DMSO (n = 9) |
| 6C | calcium | Mann- | −60 mV: p = 0.2523 | CBD3063 (n = 7) |
| | current | Whitney | −50 mV: p > 0.9999 | |
| | density | tests | −40 mV: p = 0.2991 | |
| | | | −30 mV: p = 0.4079 | |
| | | | −20 mV: p = 0.2523 | |
| | | | −10 mV: p = 0.5885 | |
| | | | 0 mV: p = 0.8371 | |
| | | | 10 mV: p = 0.8371 | |
| | | | 20 mV: p = 0.9387 | |
| | | | 30 mV: p > 0.9999 | |
| | | | 40 mV: p = 0.9182 | |
| | | | 50 mV: p = 0.5360 | |
| | | | 60 mV: p > 0.9999 | |
| FIG. | Peak | Mann- | DMSO (0.1%) vs. | DMSO (n = 9) |
| 6D | P/Q-type | Whitney | CBD3063 (20 μM): | CBD3063 (n = 7) |
| | calcium | test | p = 0.8371 | |
| | current | | | |
| | density | | | |
| FIG. | R-type | Multiple | −70 mV: p = 0.5805 | DMSO (n = 7) |
| 6E | calcium | Mann- | −60 mV: p = 0.7739 | CBD3063 (n = 11) |
| | current | Whitney | −50 mV: p = 0.1042 | |
| | density | tests | −40 mV: p = 0.4789 | |
| | | | −30 mV: p = 0.7242 | |
| | | | −20 mV: p = 0.8601 | |
| | | | −10 mV: p = 0.2854 | |
| | | | 0 mV: p = 0.3283 | |
| | | | 10 mV: p = 0.6590 | |
| | | | 20 mV: p > 0.9999 | |
| | | | 30 mV: p > 0.9999 | |
| | | | 40 mV: p = 0.3750 | |
| | | | 50 mV: p = 0.5360 | |
| | | | 60 mV: p = 0.1317 | |
| FIG. | Peak | Mann- | DMSO (0.1%) vs. | DMSO (n = 7) |
| 6F | R-type | Whitney | CBD3063 (20 μM): | CBD3063 (n = 11) |
| | calcium | test | p = 0.6590 | |
| | current | | | |
| | density | | | |
| FIG. | T-type | Multiple | −70 mV: p = 0.0821 | DMSO (n = 11) |
| 6G | calcium | Mann- | −65 mV: p = 0.0845 | CBD3063 (n = 10) |
| | current | Whitney | −60 mV: p = 0.0720 | |
| | density | tests | −55 mV: p = 0.0341 | |
| | | | −50 mV: p = 0.0986 | |
| | | | −45 mV: p = 0.0716 | |
| | | | −40 mV: p = 0.0411 | |
| | | | −35 mV: p = 0.1971 | |
| | | | −30 mV: p = 0.9177 | |
| | | | −25 mV: p = 0.7045 | |
| | | | −20 mV: p = 0.5572 | |
| | | | −15 mV: p = 0.3144 | |
| | | | −10 mV: p = 0.0986 | |
| | | | −5 mV: p = 0.0514 | |
| | | | 0 mV: p = 0.1321 | |
| | | | 5 mV: p = 0.2512 | |
| | | | 10 mV: p = 0.3867 | |
| | | | 15 mV: p = 0.5573 | |
| | | | 20 mV: p = 0.5116 | |
| | | | 25 mV: p = 0.5573 | |
| | | | 30 mV: p = 0.6540 | |
| | | | 35 mV: p = 0.8633 | |
| | | | 40 mV: p = 0.9725 | |
| | | | 45 mV: p = 0.9725 | |
| | | | 50 mV: p = 0.9177 | |
| | | | 55 mV: p = 0.7952 | |
| | | | 60 mV: p = 0.8094 | |
| FIG. | Peak | Mann- | DMSO (0.1%) vs. | DMSO (n = 11) |
| 6H | T-type | Whitney | CBD3063 (20 μM): | CBD3063 (n = 10) |
| | calcium | test | p = 0.3144 | |
| | current | | | |
| | density | | | |
| FIG. | Resting | Mann- | DMSO (0.1%) vs. | DMSO (n = 6) |
| 7B | membrane | Whitney | CBD3063 (20 μM): | CBD3063 (n = 6) |
| | potential | test | p = 0.1147 | |
| FIG. | Rheobase | Mann- | DMSO (0.1%) vs. | DMSO (n = 6) |
| 7C | | Whitney | CBD3063 (20 μM): | CBD3063 (n = 6) |
| | | test | p = 0.0087 | |
| FIG. | Evoked | Multiple | 0 pA: p > 0.9999 | DMSO (n = 6) |
| 7D | action | Mann- | 10 pA: p = 0.0152 | CBD3063 (n = 6) |
| | potentials | Whitney | 20 pA: p = 0.0022 | |
| | per step | tests | 30 pA: p = 0.0022 | |
| | | | 40 pA: p = 0.0022 | |
| | | | 50 pA: p = 0.0043 | |
| | | | 60 pA: p = 0.0043 | |
| | | | 70 pA: p = 0.0043 | |
| | | | 80 pA: p = 0.0108 | |
| | | | 90 pA: p = 0.0043 | |
| | | | 100 pA: p = 0.0108 | |
| | | | 110 pA: p = 0.0108 | |
| | | | 120 pA: p = 0.0065 | |
| FIG. | Paw | Multiple | Pre-drug: | DMSO (n = 6) |
| 8A | with- | Mann- | p = 0.6177 | CBD3063 (n = 10) |
| | drawal | Whitney | 0.5 h: p = 0.044 | |
| | threshold | tests | 1 h: p = 0.0001 | |
| | (g) | | 2 h: p = 0.0001 | |
| | single i.t. | | 3 h: p = 0.0003 | |
| | injection | | 4 h: p = 0.0001 | |
| | | | 5 h: p = 0.0001 | |
| | | | 6 h: p = 0.0003 | |
| | | | 12 h: p = 0.0001 | |
| | | | 24 h: p = 0.0072 | |
| | | | 36 h: p = 0.0029 | |
| FIG. | Area | Mann- | DMSO (0.1%) vs. | DMSO (n = 6) |
| 8B | under the | Whitney | CBD3063 (20 μM): | CBD3063 (n = 10) |
| | curve | test | p = 0.0002 | |
| | single i.t. | | | |
| | injection | | | |
| FIG. | Paw | Multiple | Pre-SNI: p = 0.905 | DMSO (n = 5) |
| 8C | with- | Mann- | 0 d: p = 0.286 | CBD3063 (n = 5) |
| | drawal | Whitney | 1 d: p = 0.008 | |
| | threshold | tests | 3 d: p = 0.008 | |
| | (g) | | 5 d: p = 0.008 | |
| | repeated | | 7 d: p = 0.008 | |
| | i.t. | | 10 d: p = 0.008 | |
| | injections | | 14 d: p = 0.008 | |
| FIG. | Area | Mann- | DMSO (0.1%) vs. | DMSO (n = 5) |
| 8D | under the | Whitney | CBD3063 (20 μM): | CBD3063 (n = 5) |
| | curve | test | p = 0.0079 | |
| | repeated | | | |
| | i.t. | | | |
| | injections | | | |
| FIG. | Phospho- | Mann- | DMSO (0.1%) vs. | DMSO (n = 4) |
| 12B | rylation | Whitney | CBD3063 (20 μM): | CBD3063 (n = 4) |
| | of | test | p = 0.4857 | |
| | CRMP2 at | | | |
| | Ser522 | | | |
| FIG. | Phospho- | Mann- | DMSO (0.1%) vs. | DMSO (n = 4) |
| 12D | rylation | Whitney | CBD3063 (20 μM): | CBD3063 (n = 4) |
| | of | test | p = 0.3429 | |
| | CRMP2 at | | | |
| | Thr514 | | | |
| FIG. | Phospho- | Mann- | DMSO (0.1%) vs. | DMSO (n = 4) |
| 12F | rylation | Whitney | CBD3063 (20 μM): | CBD3063 (n = 4) |
| | of | test | p = 0.8857 | |
| | CRMP2 at | | | |
| | Thr555 | | | |
| FIG. | Sodium | Multiple | −70 mV: p = 0.1930 | DMSO (n = 9) |
| 13B | current | Mann- | −65 mV: p = 0.8078 | CBD3063 (n = 12) |
| | density | Whitney | −60 mV: p = 0.2188 | |
| | | tests | −55 mV: p = 0.1479 | |
| | | | −50 mV: p = 0.2773 | |
| | | | −45 mV: p = 0.0585 | |
| | | | −40 mV: p = 0.5078 | |
| | | | −35 mV: p = 0.7021 | |
| | | | −30 mV: p = 0.9722 | |
| | | | −25 mV: p = 0.4221 | |
| | | | −20 mV: p = 0.4221 | |
| | | | −15 mV: p = 0.2773 | |
| | | | −10 mV: p = 0.6016 | |
| | | | −5 mV: p = 0.8621 | |
| | | | 0 mV: p = 0.9170 | |
| | | | 5 mV: p = 0.8621 | |
| | | | 10 mV: p = 0.9170 | |
| | | | 15 mV: p = 0.9722 | |
| | | | 20 mV: p = 0.9170 | |
| | | | 25 mV: p = 0.6016 | |
| | | | 30 mV: p = 0.4221 | |
| | | | 35 mV: p = 0.4221 | |
| | | | 40 mV: p = 0.3100 | |
| | | | 45 mV: p = 0.1930 | |
| | | | 50 mV: p = 0.1930 | |
| | | | 55 mV: p = 0.1694 | |
| | | | 60 mV: p = 0.1694 | |
| FIG. | Peak | Mann- | DMSO (0.1%) vs. | DMSO (n = 9) |
| 13C | sodium | Whitney | CBD3063 (20 μM): | CBD3063 (n = 12) |
| | current | test | p > 0.9999 | |
| | density | | | |
| FIG. | Potassium | Multiple | −80 mV: p = 0.6607 | DMSO (n = 10) |
| 14B | current | Mann- | −70 mV: p = 0.5490 | CBD3063 (n = 9) |
| | density | Whitney | −60 mV: p = 0.9682 | |
| | | tests | −50 mV: p = 0.2775 | |
| | | | −40 mV: p = 0.9863 | |
| | | | −30 mV: p = 0.7802 | |
| | | | −20 mV: p = 0.7197 | |
| | | | −10 mV: p = 0.4967 | |
| | | | 0 mV: p = 0.6038 | |
| | | | 10 mV: p > 0.9999 | |
| | | | 20 mV: p = 0.9682 | |
| | | | 30 mV: p > 0.8421 | |
| | | | 40 mV: p = 0.8421 | |
| | | | 50 mV: p = 0.9682 | |
| | | | 60 mV: p = 0.9048 | |
| FIG. | Peak | Mann- | DMSO (0.1%) vs. | DMSO (n = 10) |
| 14C | potassium | Whitney | CBD3063 (20 μM): | CBD3063 (n = 9) |
| | current | test | p = 0.9048 | |
| | density | | | |

### Example 15: Results and Discussion of Examples 1-14

Stability Analysis of CBD3 Peptide Predicts A₁R₂ Dipeptide as a Suitable Target for Small Molecule Peptidomimetics

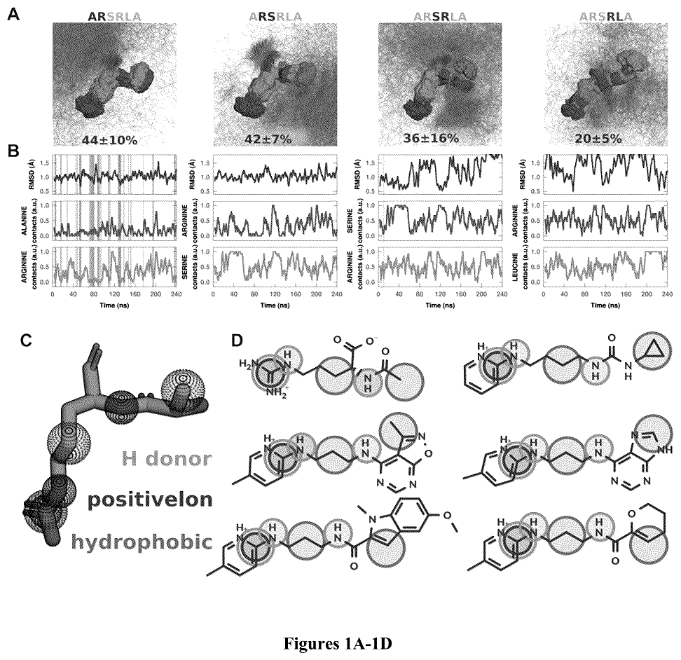

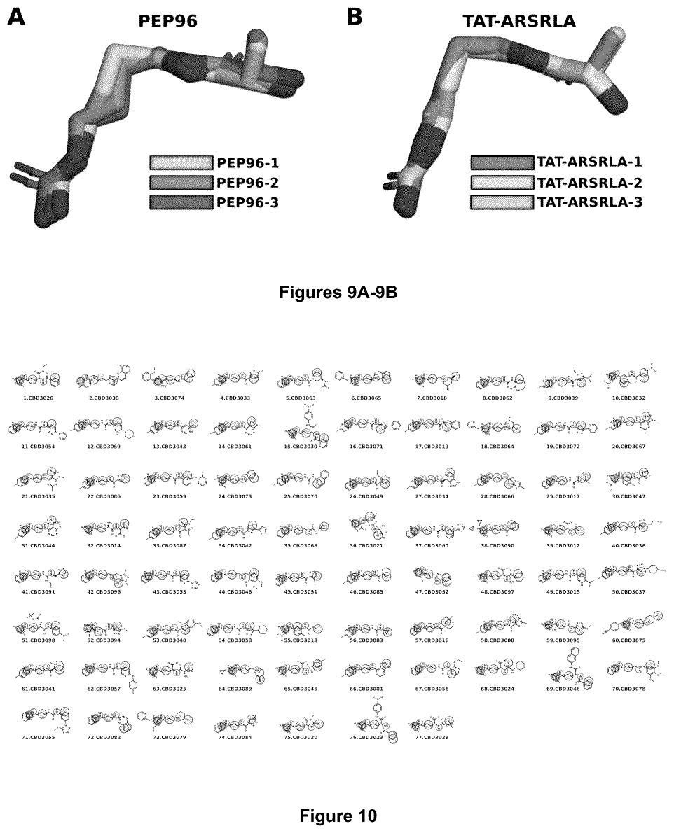

Underlying molecular recognition is the ability of a ligand to present a suitable structural motif for at least a few nanoseconds to efficiently engage its receptor (Rajamani et al, 2004). In the far-western assay using 15-mer peptides (overlapping by 12 amino acids) of full-length CRMP2 revealed that the highest binding to Caᵥ2.2 was attained by peptide ARSRLAELRGVPRGL (CBD3) (Brittain et al., 2011b). Subsequent work showed that the first six amino acids ARSRLA were critical for binding, mutagenesis suggested that two residues (Ala₁ and Arg₄) were important for binding (Moutal et al., 2018). To predict the recognition motif in CBD3, three independent molecular dynamics simulations (MDS) of the full peptide and of the TAT conjugated peptide were performed and the trajectories for the most stable dipeptide conformation (FIGS. 1A, 9A, and 9B) were scanned, it was also assessed whether the corresponding side chains were blocked by intra-peptide contacts or were exposed to solvent and free to interact (FIG. 1B). Simulations showed that A₁R₂ dipeptide, formed the most stable solvent exposed motif, while the rest of the peptide was mostly cluttered (FIGS. 1A, 1B).

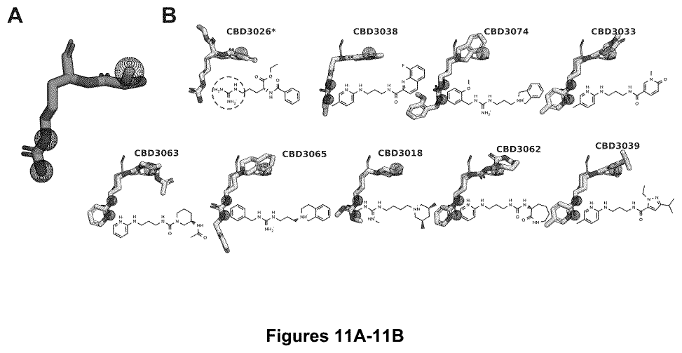

The A₁R₂ structural motif shown in FIG. 1A, defined as the conformation having the largest 1 Å radius cluster of snapshots in our MDS, encompassed about 44% of the total runs. Furthermore, since previously reported cell-based experiments also involved the cell-penetrating tat sequence conjugated to CRMP2 derived peptides, the MDS was repeated on the full tat-ARSRLA sequence, obtaining the same structural motif as for CBD3 (FIGS. 9A-9B). The robustness of the A₁R₂ motif led to its use as the basis for the design of the pharmacophore model shown in FIG. 1C. This design was entered in the open access server ZincPharmer (http://zincpharmer.csb.pitt.edu/) to search for suitable compounds among more than 27 million commercially available compounds from the ZINC database (Sterling & Irwin, 2015), obtaining ˜200 hits. Based on availability and manual curation, 77 compounds were selected for experimental validation (FIG. 1D). FIG. 10 shows the full list of compounds.

### Compound Screening in Sensory Neurons

The fact that targeting the Caᵥ2.2-CRMP2 interaction with tat-CBD3 (Brittain et al., 2011b) or myr-tat-CBD3 (Francois-Moutal et al., 2015) peptides results in reduction of depolarization-induced Ca²⁺-influx in sensory neurons was previously reported. Here, this approach was used for experimental validation. For this, Fura 2-AM-based ratiometric Ca²⁺-imaging was used in rat DRG neurons. To activate high-voltage-activated (HVA) Ca²⁺ channels, DRG neurons from all sizes were challenged with 90 mM KCl (Gomez et al., 2022). The fact that acute application (5-30 minutes) of 20 μM of myr-tat-CBD3 peptide was unable to inhibit Ca²⁺ influx have been previously demonstrated. In contrast, chronic application (˜24 hours) of 20 μM of myr-tat-CBD3 peptide inhibited Ca²⁺ currents by ˜40% (Francois-Moutal et al., 2015). Hence, overnight incubation of 20 μM of test compounds was utilized to assess the activity of these compounds.

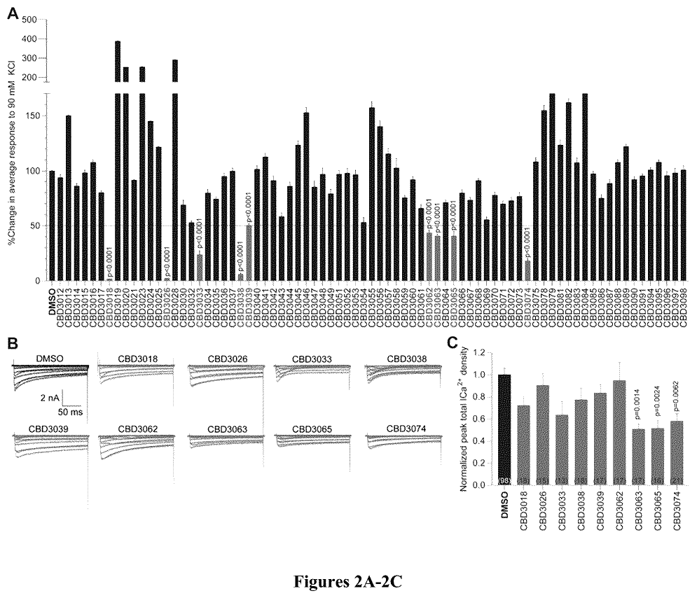

The in vitro screening showed that stimulation of DRG neurons with 90 mM KCl led to an increase in Ca²⁺ influx as shown in the control group (0.05% DMSO; FIG. 2A). Overnight incubation with 20 μM of the test compounds revealed that, of the 77 compounds tested, nine of them (CBD3018, 3026, 3033, 3038, 3039, 3062, 3063, 3065, and 3074) inhibited Ca²⁺ influx by more than 50% relative to the DMSO-treated (i.e., control) group (FIG. 2A). With one exception (CBD3026), all identified antagonists contained similar chemotypes—a protonated moiety and two dimethylamines, as well as the alanine hydrophobic moiety (FIGS. 11A-11B). Indeed, all nine compounds can be assigned to only two chemical classes and three chemotypes. Specifically, CBD3018, 3026, 3062, 3065, and 3074 belong to guanidines, CDB 3033, 3038, and 3039 feature 2-aminopyridylpropylcarboxamide class, and CBD3062 and 3063 are of analogous 2-aminopyridylpropylurea chemotype. Overall, these results showed that these first-in-class compounds predicted to disrupt Caᵥ2.2-CRMP2 interaction decrease HVA channel activity in DRG neurons from all sizes and share chemically similar motifs.

### CBD3 Compounds Inhibit Total Ca²⁺ Currents in Sensory Neurons

The calcium imaging results do not discriminate between small, medium, and large diameter DRG neurons, therefore the effects of the top compounds were next dissected on a subpopulation of small-to-medium diameter nociceptive neurons (Basbaum et al., 2009) since large-diameter DRG neurons in normal conditions transduce mechanical and proprioceptive information. The medium and small DRG neurons belong to lightly myelinated Aδ and unmyelinated C fibers, respectively. These primary afferent fibers are necessary for pain transmission since they send nociceptive information to the dorsal horn of the spinal cord (Basbaum et al., 2009); thus, whole-cell patch-clamp recordings were performed in small-to-medium diameter DRG neurons from female rats to electrophysiologically validate the nine top compounds that blocked Ca²⁺ influx.

DRGs were treated overnight with 20 μM of test compounds or vehicle (0.1% DMSO). Inward current through Ca²⁺ channels was carried by Ba²⁺ and will be referred to it as Ca²⁺ currents. From a holding potential of −90 mV, 200-ms depolarization steps from −70 to +60 mV in 10 mV increments, a family of Ca²⁺ currents was elicited (FIG. 2B). The peak current density was next obtained and it was normalized to the control group (FIG. 2C). It was found that CBD3063, 3065, and 3074 significantly reduced total Ca²⁺ currents when compared to cells treated with 0.1% DMSO (DMSO: 1.00±0.06 pA/pF; CBD3063: 0.51±0.04 pA/pF; CBD3065: 0.52±0.07 pA/pF; CBD3074: 0.58±0.06 pA/pF).

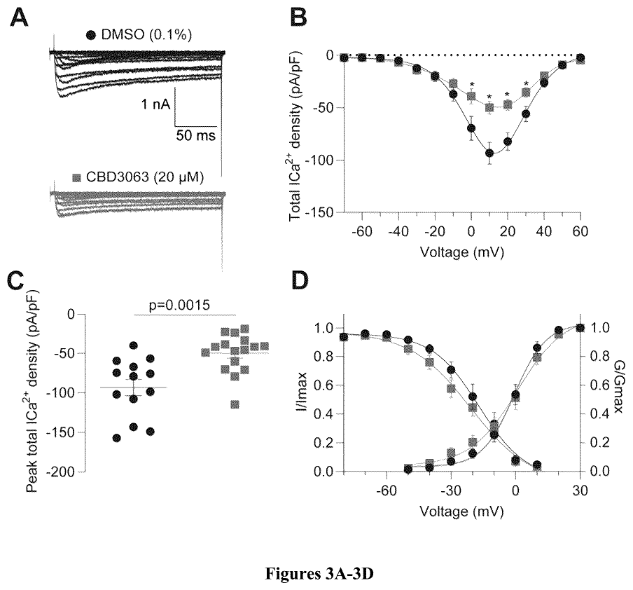

Of the three compounds that inhibited Ca²⁺ currents, CBD3063 and CBD3065 represented unique chemotypes (Table 2) and were selected for additional characterization. However, in preliminary tests, CBD3065 did not exhibit analgesic properties. Thus, CBD3065 was not pursued further. The current density-voltage relationships was measured and it was observed that incubation with 20 μM of CBD3063 significantly decreased Ca²⁺ current density from 0 to 30 mV (FIG. 3A, B). Furthermore, at peak current density (+10 mV; FIG. 3C), the reduction in Ca²⁺ currents imposed by CBD3063 was ˜46.54% when compared to cells treated with 0.1% DMSO (DMSO: −93.23±10.40 pA/pF; CBD3063: −49.84±6.17 pA/pF). Inspection of voltage-dependence of activation revealed no difference in the half activation potential and slope factors between groups (FIG. 3D and Table 3). The steady-state inactivation kinetics of the channels at multiple test potentials was also assessed by measuring the fraction of current remaining at +10 mV. As seen in FIG. 3D and Table 3, the results revealed no significant differences in half inactivation potential and slope factors between the conditions. Collectively, these findings indicate that the top compound—CBD3063—inhibits Ca²⁺ currents in small-to-medium diameter (i.e., presumptively nociceptive) sensory neurons.

TABLE 2
Calculated Properties of Compounds Inhibiting Ca⁺² Influx by More Than 50%

| | | Compound | | | logS | cLog | HB | HB | RO | | Rot | TPS | QE |
|---|---|---|---|---|---|---|---|---|---|---|---|---|---|
| ID | IUPAC Name | class | Mw | BBB | (7.4) | P | D | A | 5 | NHOH | B | A | D |
| CBD3018 | (Z)-N′-{4-[(3R,5S)-3,5- | Guanidines | 254.4 | 4.2 | 0.90 | 1.54 | 2 | 2 | Y | 2 | 5 | 39.7 | 0.44 |
| | dimethylpiperidin-1- | | | | | | | | | | | | |
| | yl]butyl}-N,N′′- | | | | | | | | | | | | |
| | dimethylguanidine | | | | | | | | | | | | |
| CBD3026 | ethyl N-benzoyl-(R)- | Guanidines | 306.4 | 2.2 | 0.00 | 0.61 | 4 | 4 | Y | 5 | 8 | 117.3 | 0.24 |
| | arginine | | | | | | | | | | | | |
| CBD3033 | 1-methyl-N-{3-[(5- | 2- | 300.4 | 4.0 | −1.70 | 1.32 | 2 | 5 | Y | 2 | 6 | 76.0 | 0.79 |
| | methylpyridin-2- | aminopyridylpropyl- | | | | | | | | | | | |
| | yl)amino]propyl}-6- | carboxamides | | | | | | | | | | | |
| | oxopyridine-3- | | | | | | | | | | | | |
| | carboxamide | | | | | | | | | | | | |
| CBD3038 | 8-fluoro-N-{3-[(5- | 2- | 338.4 | 4.2 | −4.10 | 3.31 | 2 | 4 | Y | 2 | 6 | 66.9 | 0.68 |
| | methylpyridin-2- | aminopyridylpropyl- | | | | | | | | | | | |
| | yl)amino]propyl}quinoline- | carboxamides | | | | | | | | | | | |
| | 2-carboxamide | | | | | | | | | | | | |
| CBD3039 | 2-ethyl-5-isopropyl-N- | 2- | 329.4 | 4.2 | −2.90 | 2.96 | 2 | 5 | Y | 2 | 8 | 71.8 | 0.73 |
| | {3-[(5-methylpyridin-2- | aminopyridylpropyl- | | | | | | | | | | | |
| | yl)amino]propyl}pyrazole- | carboxamides | | | | | | | | | | | |
| | 3-carboxamide | | | | | | | | | | | | |
| CBD3062 | 1-[(3S)-2-oxoazepan-3- | 2- | 305.4 | 2.9 | −1.90 | 0.85 | 4 | 4 | Y | 4 | 6 | 95.2 | 0.59 |
| | yl]-3-[3-(pyridin-2- | aminopyridylpropylureas | | | | | | | | | | | |
| | ylamino)propyl]urea | | | | | | | | | | | | |
| CBD3063 | (3R)-3-acetamido-N-[3- | 2- | 319.4 | 3.8 | −2.00 | 1.19 | 3 | 4 | Y | 3 | 6 | 86.4 | 0.69 |
| | (pyridin-2- | aminopyridylpropylureas | | | | | | | | | | | |
| | ylamino)propyl]piperidine- | | | | | | | | | | | | |
| | 1-carboxamide | | | | | | | | | | | | |
| CBD3065 | N′-benzyl-N-[3-(3,4- | Guanidines | 322.5 | 4.6 | 0.00 | 2.75 | 3 | 2 | Y | 3 | 6 | 51.2 | 0.44 |
| | dihydro-1H-isoquinolin- | | | | | | | | | | | | |
| | 2-yl)propyl]guanidine | | | | | | | | | | | | |
| CBD3074 | N-[3-(1,3- | Guanidines | 338.5 | 4.2 | 0.00 | 2.72 | 3 | 3 | Y | 3 | 7 | 60.4 | 0.41 |
| | dihydroisoindol-2- | | | | | | | | | | | | |
| | yl)propyl]-N′-[(2- | | | | | | | | | | | | |
| | methoxyphenyl)methyl] | | | | | | | | | | | | |
| | guanidine | | | | | | | | | | | | |

Compounds identified as active in calcium imaging (FIG. 2A). Mw, molecular weight (Da); BBB score, indicates probability of compound having CNS exposure where scores in the range [4-6] correctly predicted 90.3% of CNS drugs (Gupta, Lee, Barden & Weaver, 2019); LogS (7.4), predicted solubility (M) at pH 7.4; cLogP, predicted lipophilicity coefficient in octanol/water; HBD, number of hydrogen-bond donors; HBA, number of hydrogen bond acceptors; RO5, binary (Y/N) assignment of complying with Lipinski rule-of-5 (Lipinski, 2004); NHOH, number of polar NH and OH hydrogens; RotB, number of rotatable bonds; TPSA, topological polar surface area (Å²); QED, Quantitative Estimate of Druglikeness where a score of 1 indicates all properties are favorable (Bickerton, Paolini, Besnard, Muresan & Hopkins, 2012). Properties calculated with RDKit and ChemAxon modules.

TABLE 3
Gating Properties of Ionic Currents Recorded From Rat
DRG Neurons in the Presence of CBD3063

| | |
|---|---|
| DMSO | CBD3063 |

Total Ca²⁺ currents

| | | |
|---|---|---|
| Activation | | |
| V_{1/2} | −0.665 ± 1.091 (13) | 0.971 ± 2.338 (16) |
| k | 7.429 ± 0.991 (13) | 11.004 ± 2.181 (16) |
| Inactivation | | |
| V_{1/2} | −17.081 ± 2.647 (13) | −20.944 ± 3.182 (16) |
| k | −10.916 ± 2.186 (13) | −14.132 ± 2.880 (16) |

N-type Ca²⁺ currents

| | | |
|---|---|---|
| Activation | | |
| V_{1/2} | −5.993 ± 0.680 (12) | −5.275 ± 0.669 (17) |
| k | 5.266 ± 0.570 (12) | 5.616 ± 0.566 (17) |
| Inactivation | | |
| V_{1/2} | −28.936 ± 2.206 (12) | −31.129 ± 2.169 (17) |
| k | −15.101 ± 2.011 (12) | −17.247 ± 2.104 (17) |

L-type Ca²⁺ currents

| | | |
|---|---|---|
| Activation | | |
| V_{1/2} | −3.677 ± 0.791 (8) | −2.704 ± 0.710 (7) |
| k | 5.797 ± 0.678 (8) | 5.528 ± 0.615 (7) |
| Inactivation | | |
| V_{1/2} | −10.531 ± 0.875 (8) | −10.128 ± 2.133 (7) |
| k | −4.512 ± 0.848 (8) | −4.961 ± 1.976 (7) |

P/Q-type Ca²⁺ currents

| | | |
|---|---|---|
| Activation | | |
| V_{1/2} | −3.703 ± 0.987 (9) | −3.265 ± 0.824 (7) |
| k | 5.375 ± 0.835 (9) | 4.186 ± 0.663 (7) |
| Inactivation | | |
| V_{1/2} | −18.283 ± 1.478 (9) | −18.667 ± 1.688 (7) |
| k | −7.593 ± 1.287 (9) | −9.396 ± 1.470 (7) |

R-type Ca²⁺ currents

| | | |
|---|---|---|
| Activation | | |
| V_{1/2} | −8.118 ± 0.842 (7) | −4.098 ± 0.693 (11) |
| k | 6.493 ± 0.736 (7) | 7.244 ± 0.579 (11) |
| Inactivation | | |
| V_{1/2} | −23.745 ± 3.57 (7) | −16.840 ± 2.186 (11) |
| k | −13.155 ± 3.325 (7) | −11.870 ± 1.850 (11) |

T-type Ca²⁺ currents

| | | |
|---|---|---|
| Activation | | |
| V_{1/2} | −16.859 ± 0.722 (11) | −10.982 ± 1.992 (10) |
| k | 5.736 ± 0.636 (11) | 9.924 ± 1.628 (10) |
| Inactivation | | |
| V_{1/2} | −41.506 ± 1.708 (11) | −35.122 ± 2.752 (10) |
| k | −9.884 ± 1.585 (11) | −12.929 ± 2.490 (10) |

Na⁺ currents

| | | |
|---|---|---|
| Activation | | |
| V_{1/2} | −23.336 ± 0.663 (9) | −21.738 ± 0.852 (12) |
| k | 3.514 ± 0.576 (9) | 4.749 ± 0.744 (12) |
| Inactivation | | |
| V_{1/2} | −37.602 ± 3.112 (9) | −32.552 ± 2.084 (12) |
| k | −16.081 ± 3.278 (9) | −12.003 ± 1.936 (12) |

Values are means±SEM calculated from fits of the data from the indicated number of individual cells (in parentheses) to the Boltzmann equation; V_{1/2} midpoint potential (mV) for voltage-dependent of activation or inactivation; k, slope factor. These values pertain to FIGS. 3A-D, 5A-E, 6A-H, and 13A-D. Data were analyzed with Mann-Whitney test. DRG is dorsal root ganglia; DMSO is dimethyl sulfoxide.
CBD3063 Disrupts Caᵥ2.2-CRMP2 Binding and Impairs Membrane Trafficking of Caᵥ2.2 Channels Without Effecting CRMP2 Phosphorylation

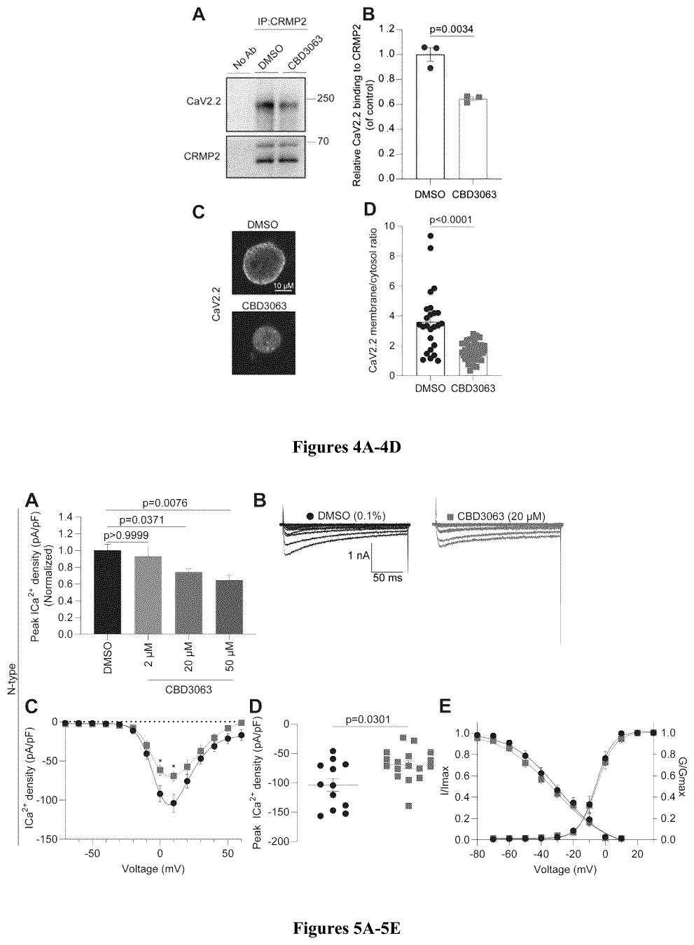

tat-CBD3 (Brittain et al., 2011b) and myr-tat-CBD3 (Francois-Moutal et al., 2015) peptides were previously reported to inhibit the Caᵥ2.2-CRMP2 interaction and reduced surface expression of Caᵥ2.2. Therefore, it was next determined whether CBD3 peptidomimetic CBD3063 could interfere with Caᵥ2.2-CRMP2 binding and affect the membrane localization of Caᵥ2.2 channels. For this, catecholamine A differentiated (CAD) cells—a mouse neuronal cell line expressing both CRMP2 and Caᵥ2.2—were incubated overnight with 0.1% DMSO or 20 μM CBD3063. Immunoprecipitation assays revealed an ˜35% reduction in the level of Caᵥ2.2 protein immunoprecipitated by CRMP2 in cells treated with CBD3063 versus control (FIGS. 4A and 4B; DMSO: 1.00±0.05; CBD3063: 0.64±0.02). These results provided evidence that CBD3063 inhibits the association between Caᵥ2.2 and CRMP2.

CRMP2 was previously reported to facilitate the trafficking of Caᵥ2.2 to the plasma membrane (Brittain, Piekarz, Wang, Kondo, Cummins & Khanna, 2009; Chi et al., 2009) and that cell-penetrant CBD3 peptides (Brittain et al., 2011b; Francois-Moutal et al., 2015; Xie et al., 2016) decrease surface trafficking of Caᵥ2.2. Having established that CBD3063 can inhibit Caᵥ2.2-CRMP2 interaction in vitro, It was next determined whether CBD3063 alters the subcellular localization of Caᵥ2.2 channels. To evaluate this, DRG neurons were incubated with vehicle (0.1% DMSO) or CBD3063 (20 μM) overnight and subjected to immunofluorescent microscopy to assess the membrane expression of these channels. In control DRGs treated with vehicle, the fluorescent signal for Caᵥ2.2 presents as an annulus at the plasma membrane (FIG. 4C). As illustrated in FIG. 4D, Caᵥ2.2 expression in the membrane was significantly decreased when cells were incubated with CBD3063 (DMSO: 3.59±0.42; CBD3063: 1.64±0.10). These data suggested that by uncoupling Caᵥ2.2-CRMP2 interaction, CBD3063 reduced surface trafficking of Caᵥ2.2 channels to the plasma membrane of sensory neurons.

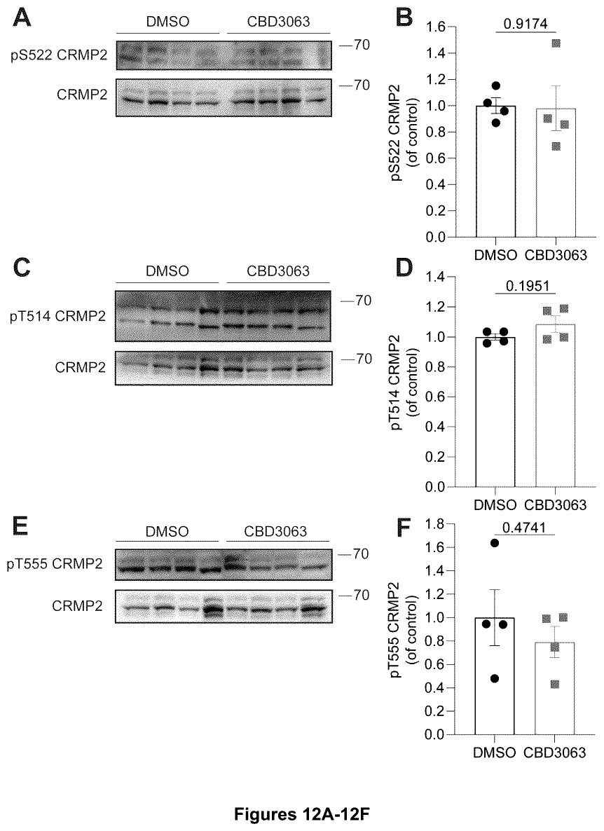

Because the cellular functions of CRMP2 were mediated by its phosphorylation, it was determined whether CBD3063 could alter the phosphorylation states of CRMP2. Immunoblot analyses of lysates prepared from CAD cells exposed to overnight incubation of CBD3063 (20 mM) showed no differences in the expression of phosphorylated CRMP2 at sites 522 (by cyclin dependent kinase 5 (Cdk5); FIGS. 12A and 12B), 514 (by glycogen synthase kinase-3 beta (GSK-3β); FIGS. 12C and 12D), or 555 (RhoK; FIGS. 12E and 12F). These results rule out a potential side effect of CBD3063 on the phosphorylation status CRMP2.

CBD3063 Selectively Decreases Caᵥ2.2 (N-type) Ca²⁺ Currents in Sensory Neurons

It was next determined whether the CBD3063-mediated decrease in Ca²⁺ currents was due to our target Caᵥ2.2 (N-type) channels. To test the minimum concentration needed to inhibit N-type Ca²⁺ channels' function, small-to-medium sized DRG neurons were treated overnight with 2, 20, and 50 μM of CBD3063 or control (0.1% DMSO). The next day, whole-cell patch-clamp recordings were performed in the presence of a cocktail of blockers of all other subtypes of calcium channels present in DRG neurons, thus isolating the N-type currents. When compared to DMSO-treated cells, 2 μM of CBD3063 did not modify the activity of these channels. On the contrary, 20 and 50 μM of CBD3063 significantly reduced peak current density, thus, 20 μM was sufficient to decrease N-type currents and used for further experimentation (DMSO: 1.00±0.07; 2 μM CBD3063: 0.93±1.07; 20 μM CBD3063: 0.74±0.04; 50 μM CBD3063: 0.65±0.06; FIG. 5A). A significant decrease in N-type currents (FIG. 5B) and current density at 0 and +10 mV (FIG. 5C) was observed in cells incubated with 20 μM of CBD3063. At peak current density (+10 mV; FIG. 5D), CBD3063 decreased N-type currents by ˜33.5% when compared to the control group (DMSO: −104.00±11.32 pA/pF; CBD3063: −69.18±6.54 pA/pF). Plotting voltage-dependence of activation and steady-state inactivation curves revealed no differences in half activation or inactivation potentials and slope factors between these two conditions (FIG. 5E and Table 3). These data confirm that targeting the CRMP2-Caᵥ2.2 interaction with CBD3063 decreases current influx through N-type Ca²⁺ channels.

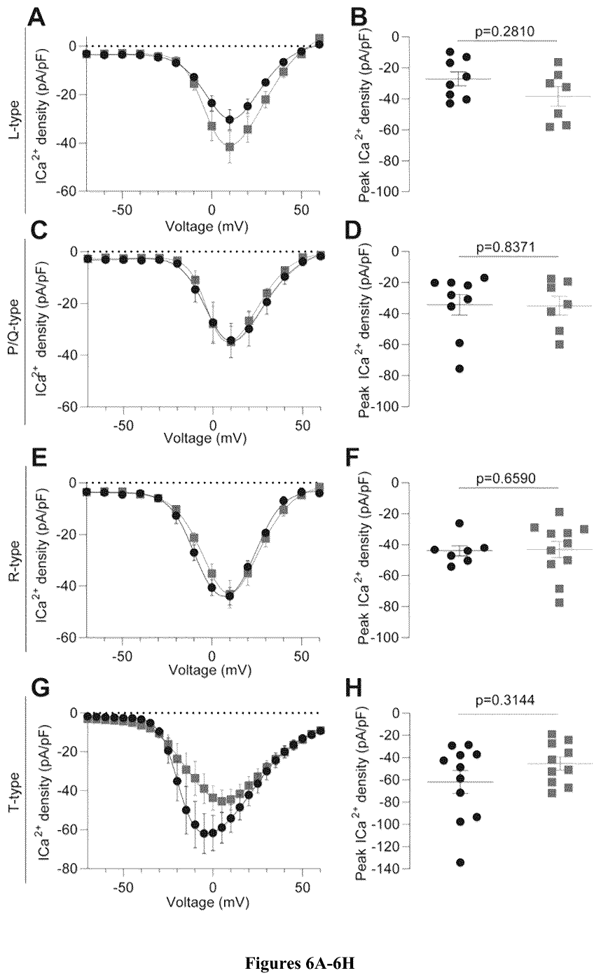

Furthermore, to test any off-target effects of CBD3063 current density-voltage curves and peak current densities for Caᵥ1 (L-type; FIG. 6A, 6B), Caᵥ2.1 (P/Q-type; FIG. 6C, 6D), Caᵥ2.3 (R-type; FIG. 6E, 6F), and Caᵥ3 (T-type; FIG. 6G, 6H) calcium channels were measured. It was found that CBD3063 does not alter Ca²⁺ influx through these channels when compared to cells treated with 0.1% DMSO. When the voltage-dependence of activation and inactivation of these channels was explored, it was observed that CBD3063 did not alter these parameters (Table 3). These findings showed that CBD3063 selectively modulates the activity of N-type Ca²⁺ channels.

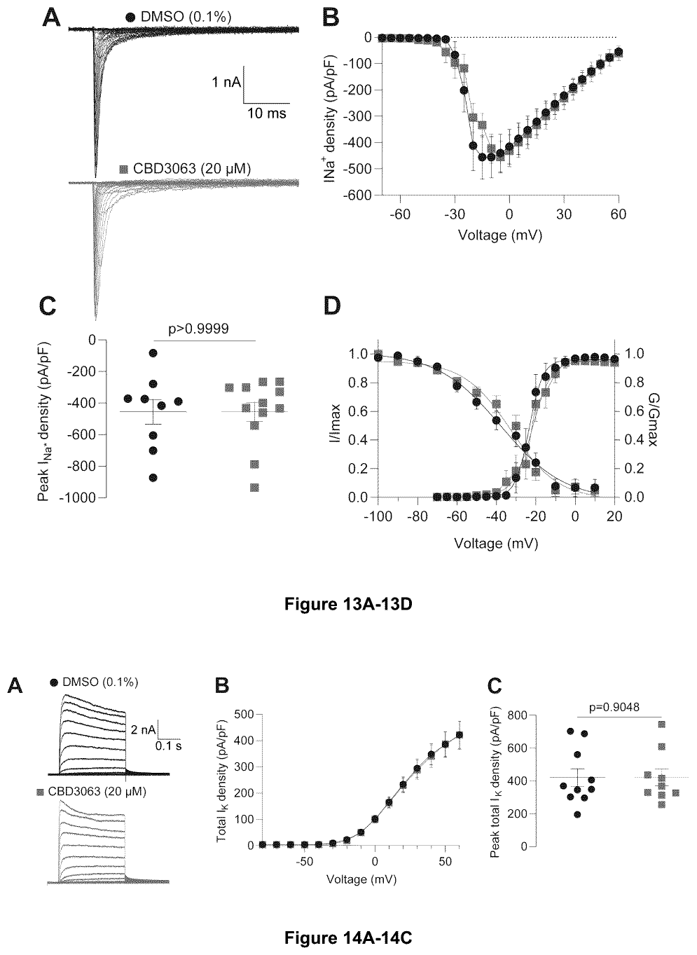

As CRMP2 has been reported to regulate the functional activity of Na⁺ channels (Dustrude, Moutal, Yang, Wang, Khanna & Khanna, 2016; Dustrude, Perez-Miller, Francois-Moutal, Moutal, Khanna & Khanna, 2017; Dustrude, Wilson, Ju, Xiao & Khanna, 2013), the ability of CBD3063 to affect Na⁺ currents in sensory neurons was tested. CDB3063 did not affect Na⁺ current density (DMSO: −455.7±78.70 pA/pF; CBD3063: −455.0±60.79 pA/pF) or gating properties of Na⁺ channels in DRG neurons (FIGS. 13A-13D and Table 3). Furthermore, it was found that CBD3063 had no effect on voltage-gated potassium channels (DMSO: 420.9±54.19 pA/pF; CBD3063: 422.1±52.70 pA/pF; FIGS. 14A-14C). Altogether, the above results demonstrated that CBD3063 specifically inhibited Caᵥ2.2, sparing all other voltage-gated ion channels expressed in DRG sensory neurons.

### CBD3063 Decreases Excitability of Rat Sensory Neurons

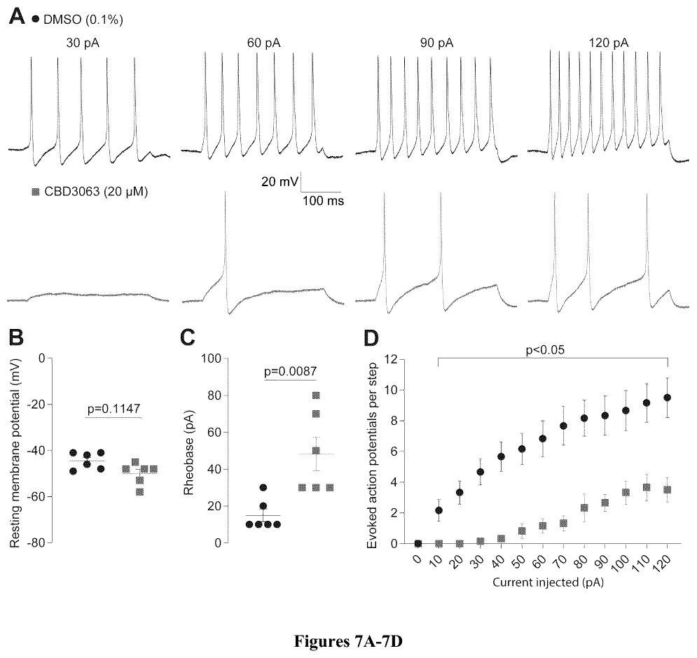

Caᵥ2.2 channels have critical roles in controlling the excitability of DRG neurons, according to earlier studies (Yang et al., 2018). Since Caᵥ2.2 populations are affected by CBD3063, the ability of CBD3063 to alter the excitability of DRG sensory neurons was investigated (FIGS. 7A-D). Action potentials (AP) were elicited in rat sensory neurons during whole cell current-clamp tests in response to current injections from 0-120 pA after overnight incubation with 0.1% DMSO or 20 μM CBD3063. Representative traces are shown in FIG. 7A. While the resting membrane potential remained unchanged between these two conditions (DMSO: −44.50±1.478 mV; CBD3063: −50.00±1.915 mV; FIG. 7B), CBD3063 significantly increased the rheobase—the minimum current necessary to evoke an AP—compared to the control (DMSO: 15.00±3.416 mV; CBD3063: 48.33±9.098 mV; FIG. 7C). CBD3063 also reduced the number of evoked APs over current injection steps ranging from 10 to 120 pA (FIG. 7D). These results show that CBD3063 mediated inhibition of the Caᵥ2.2 channels culminates to curb sensory neuron excitability.

### Disruption of Caᵥ2.2-CRMP2 Binding Confers Antinociception

In humans, targeting auxiliary subunits that regulate Caᵥ2.2 channels is effective against chronic pain. For example, targeting α2δ-1 subunit with Gabapentin and Pregabalin alleviates pain by interfering with Caᵥ2.2-α2δ-1 interaction (Bauer, Rahman, Tran-van-Minh, Lujan, Dickenson & Dolphin, 2010; Hendrich et al., 2008; Sutton, Martin, Pinnock, Lee & Scott, 2002). Consequently, it was tested whether interrupting CRMP2 binding to Caᵥ2.2 with CBD3063 could be antinociceptive in a rat model of spared nerve injury (SNI). This model of neuropathic pain results in long-lasting thermal hyperalgesia and mechanical allodynia on the affected hind paw (Decosterd & Woolf, 2000). Male rats were used for these studies since interrupting the Caᵥ2.2-CRMP2 interaction in vivo does not display sex dimorphism with equivalent effects observed in females (Ju, Li, Allette, Ripsch, White & Khanna, 2013; Ripsch, Ballard, Khanna, Hurley, White & Khanna, 2012) and males (Fischer, Pan, Vilceanu, Hogan & Yu, 2014; Francois-Moutal et al., 2015; Moutal et al., 2018).

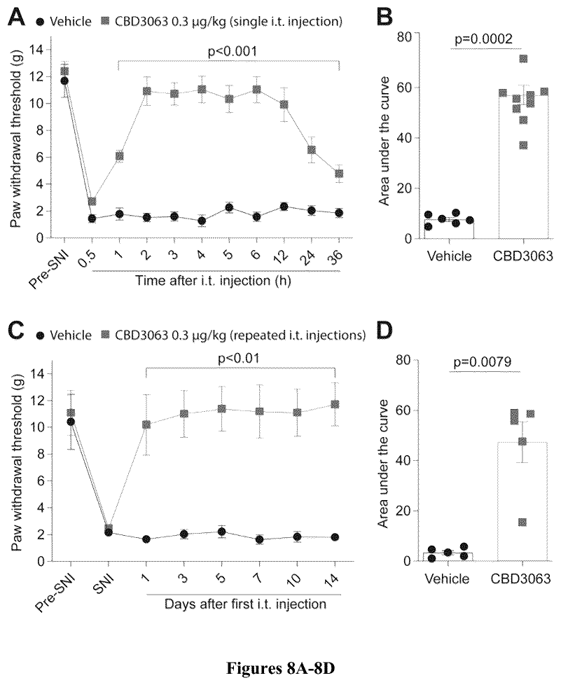

Ligation of the tibial and peroneal nerves in male rats resulted in the development of mechanical hypersensitivity (FIGS. 8A-8D). CBD3063 was administered intrathecally (0.3 μg/kg) to directly reach the site of action of Caᵥ2.2 in the SDH, where the central termini of primary afferent fibers synapse with second order neurons in the SDH. A single intrathecal administration of CBD3063, 7 days following SNI, resulted in a significant reversal of mechanical allodynia that started 60 minutes post-injection and lasted up to 36 hours (FIG. 8A). A significant increase in the area under the curve was observed after injection of CBD3063 compared to the control group (DMSO: 7.650±0.84; CBD3063: 57.05±3.84; FIG. 8B).

To assess the long-term antinociceptive effect of CBD3063, rats were injected with CBD3063 (0.3 μg/kg) starting 7 days after SNI surgery and once a day for 14 days. On days 1, 3, 5, 7, 10, and 14, paw withdrawal thresholds were determined at six hours following each injection (FIG. 8C). Repeated administration of CBD3063 resulted in a prolonged reversal of SNI—induced mechanical allodynia (FIG. 8C). A significant increase in the area under the curve was observed following treatment with CBD3063 for 14 days (DMSO: 3.37±0.86; CBD3063: 47.26±8.22; FIG. 8D). These results demonstrated that targeting the Caᵥ2.2-CRMP2 interaction resulted in a long-lasting reversal of mechanical hypersensitivity, without any tolerance (loss of antinociception), in a nerve injury model of persistent pain.

Structure-based drug design allows the optimization of a chemical structure in which the structure of a protein is used as the basis to identify or design new chemical compounds that are predicted to bind to a target (Aparoy, Reddy & Reddanna, 2012). Alternatively, ligand-based approach is used in the absence of the protein structure and relies on knowledge of molecules that bind to the biological target (Aparoy, Reddy & Reddanna, 2012). To disrupt the Caᵥ2.2-CRMP2 interaction, neither a structure of the complex nor any drug target data was previously known. Thus, based solely on the sequence of the CBD3 peptide, first principles were used to identify the structural motif that triggers this interaction. Recently, a similar problem was tackled whereby using MDS, the residue anchoring the interaction between a peptide derived from the disordered N-terminal of Kᵥ2.1 and syntaxin1a was identified, which in combination with syntaxin crystal structure led to the discovery of a first-in-class small molecule neuroprotectant (Yeh et al., 2019).

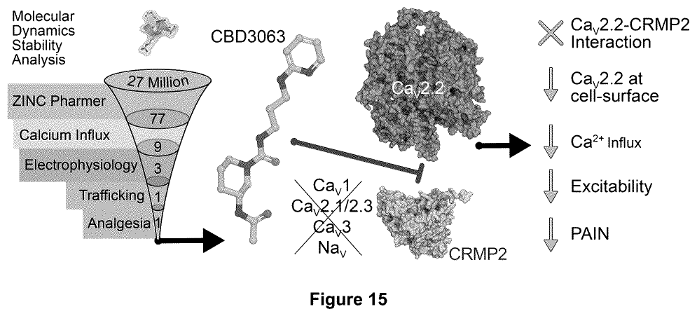

In the present disclosure, a novel pipeline was developed that leverages the computational power of MDS to identify the most stable conformational motif of a peptide, i.e., CBD3, and then used it to develop a pharmacophore model to generate peptidomimetics—a medicinal chemistry approach where parts of the peptide were successively replaced by non-peptide moieties until a non-peptide small molecule is discovered (Perez, 2018). Peptides present desirable medicinal properties like predictable metabolism, good efficacy, safety, and tolerability; however, they are chemically and physically unstable due to rapid proteolysis and inadequate membrane permeability (Fosgerau & Hoffmann, 2015). Notably, editing peptide sequences to develop peptidomimetic analogs creates a promising class of therapeutics that can have inherent advantages, including oral administration, good membrane penetration ability, and enhanced biological activity (Smith, 2015). Considering these strengths, the model predicted first-in-class compounds to disrupt the Caᵥ2.2-CRMP2 interaction (FIG. 15). Specifically, more than 27 million commercially available compounds were screened in the ZINC database using the open access server ZincPharmer and 77 suitable compounds for experimental testing were identified. The primary in vitro screening identified 9 compounds that inhibit Ca⁺² influx by more than 50% relative to control. Furthermore, analysis of the shared pharmacophores among the 9 compounds permitted to predict active chemotypes that when screened against MDS of CRMP2-derived peptides allowed to fully rationalize active from inactive peptides. The latter provides a strong rationale for this method and its potential application for other targets. From a biological point of view, it was shown that by disrupting the Caᵥ2.2-CRMP2 interaction, CBD3063: (i) inhibits Ca²⁺ influx in rat DRG neurons, (ii) decreases the functional activity of N-type Ca²⁺ channels, (iii) reduces membrane expression of Caᵥ2.2, (iv) does not affect the activity of other voltage-gated ion channels, (v) reduces sensory neuron excitability, and (vi) is antinociceptive in rats with a spared nerve injury model of pain.

Caᵥ2.2 channels are almost exclusively expressed in neuronal tissue (Nowycky, Fox & Tsien, 1985) and are abundant at presynaptic nerve terminals where they trigger the release of neurotransmitters such as glutamate, calcitonin gene-related peptide, and substance P (Evans, Nicol & Vasko, 1996) via physically interacting with the synaptic release machinery (Zamponi, 2003). For this reason, Caᵥ2.2 channels are critical determinants of increased neuronal excitability (Yang et al., 2018) and neurotransmission that accompany chronic neuropathic pain (Cizkova et al., 2002). In 2018, the Dolphin laboratory (Nieto-Rostro, Ramgoolam, Pratt, Kulik & Dolphin, 2018) reported a mouse expressing Caᵥ2.2 channels with an extracellularly accessible hemagglutinin epitope tag engineered into their pore forming Caᵥ2.2 α1 subunit (Caᵥ2.2_HA) permitting, for the first time, identification of endogenous Caᵥ2.2 channels in the plasma membrane of sensory neurons. These mice revealed disease-associated changes in the subcellular distribution of Caᵥ2.2 in the pain pathway that confirmed the importance of these channels as suitable targets for development of novel pain therapies. Consistent with these findings, the therapeutic potential of targeting Caᵥ2.2 has been demonstrated in Caᵥ2.2 deficient mice which have reduced responses to mechanical stimuli, radiant heat, and chemical-induced inflammatory pain (Hatakeyama et al., 2001; Kim et al., 2001) and in nociceptive neurons specifically expressing the exon 37a variant of Caᵥ2.2 (Caᵥ2.2e[37a]) mice that display increased N-type Ca²⁺ currents and open channel probability when compared to neurons that only express the exon 37b variant of (Caᵥ2.2e[37b]) (Bell, Thaler, Castiglioni, Helton & Lipscombe, 2004). In these mice, in vivo silencing of Caᵥ2.2e[37a] prevented the development of mechanical allodynia and thermal hyperalgesia, demonstrating that targeting Caᵥ2.2e[37a] channels by using splice isoform-specific gene silencing is an effective means for controlling the transmission of thermal and mechanical stimuli in pain conditions.

Despite compelling genetic evidence of the importance of Caᵥ2.2 in pain, clinical development of N-type Ca²⁺ channel blockers have proven to be challenging. Although modulators of Caᵥ2.2-α2δ interaction (i.e., Gabapentin and Pregabalin) are recommended as first-line treatment for neuropathic pain (Attal et al., 2006), they only partially alleviate chronic pain, are implicated in overdose deaths (Kuehn, 2022), and cause a litany of side effects (Zamponi, Striessnig, Koschak & Dolphin, 2015). A Caᵥ2.2-selective, state-dependent inhibitor—N-triazole oxindole (TROX-1)—was reported by Merck but cardiovascular and motor impairment hampered its further development (Abbadie et al., 2010). Another study reported a sulfonamide-derived, state-dependent inhibitor of Caᵥ2.2, but this compound was limited by structural liabilities of this class of compounds (Shao et al., 2012). Targeting other interacting partners of Caᵥ2.2 can also result in reversal of pain symptoms. For example, the β3 auxiliary subunits interact with Caᵥ2.2 channels (Ludwig, Flockerzi & Hofmann, 1997; Scott et al., 1996) to speed up their activation, increase their membrane localization, and increase neurotransmitter release (Richards, Butcher & Dolphin, 2004; Welling et al., 1993). Expression of β3 protein increases in neuropathic pain (Li et al., 2012) and β3-null mice exhibit suppressed pain responses due to decreased Caᵥ2.2 currents (Murakami et al., 2002). Consistent with these findings, it was reported that targeting the Caᵥ2.2-β interaction reduces currents through Caᵥ2.2 channels, inhibits spinal neurotransmission, and alleviates neuropathic pain (Khanna et al., 2019). Collectively, these studies converge on the idea that targeting auxiliary subunits of Caᵥ2.2 channels is beneficial for pain management. Along these lines, accumulating evidence points to CRMP2 as a new auxiliary subunit of Caᵥ2.2 channels (Striessnig, 2018).

Over the past decade, it has been shown that interrupting the interaction between Caᵥ2.2 and CRMP2 with CBD3 peptides is efficacious in reversing pain. It was reported that disrupting Caᵥ2.2-CRMP2 binding with tat-CBD3 or myr-tat-CBD3 peptides does not affect memory, motor functions, or anxiety/depression, and does not produce any addictive behaviors (Brittain et al., 2011b; Francois-Moutal et al., 2015). Likewise, interrupting this interaction has neuroprotective effects (Brittain et al., 2011a; Brustovetsky, Pellman, Yang, Khanna & Brustovetsky, 2014; Ji et al., 2019). Tat-CBD3 (Brittain et al., 2011a; Brustovetsky, Pellman, Yang, Khanna & Brustovetsky, 2014) and R9-CBD3 (Ji et al., 2019) also disrupt the CRMP2-NMDAR interaction. As a result of this, both peptides attenuate NMDAR-mediated currents, have neuroprotective effects against glutamate-induced Ca²⁺ dysregulation (Brittain et al., 2011a; Brustovetsky, Pellman, Yang, Khanna & Brustovetsky, 2014) and protect against neurotoxicity caused the toxic fragment of amyloid-β (Aβ)₂₅₋₃₅ (Ji et al., 2019). These studies demonstrate that interfering with Caᵥ2.2-CRMP2 binding is safe, beneficial, and does not produce unwanted side effects.

A single amino acid point mutant of CBD3 (tat-CBD3_{A6K}) was previously reported to decrease R—and T-type Ca²⁺ currents in DRG neurons (Piekarz et al., 2012). tat-CBD3 peptide was also reported to decrease T-type currents (Piekarz et al., 2012) in sensory neurons. Importantly, in the present disclosure, CBD3063 did not exert an effect on any other voltage-gated calcium channel, which argues for selectivity of the peptidomimetic compound for Caᵥ2.2. This is in line with previous observations that the activity of low-voltage-activated Ca²⁺ channels is independent of CRMP2 (Cai, Shan, Zhang, Moutal & Khanna, 2019). Cdk5-mediated CRMP2 phosphorylation at residue S522 have shown to increase its binding to Caᵥ2.2, which leads to an increase in calcium influx (Brittain, Wang, Eruvwetere & Khanna, 2012). To discard a potential effect of CBD3063 on CRMP2 phosphorylation, pS522 CRMP2 was measured and it was found that CBD3063 did not affect this posttranslational modification. This data correlates with the previous findings that interrupting CaV2.2-CRMP2 interaction with myr-tat-CBD3 does not affect CRMP2 phosphorylation (Francois-Moutal et al., 2015). Along the same lines, the regulation of sodium channels by CBD3063 was investigated since the phosphorylation and SUMOylation of CRMP2 was shown to regulate Nav1.7 channel trafficking and activity (Dustrude, Moutal, Yang, Wang, Khanna & Khanna, 2016; Dustrude, Perez-Miller, Francois-Moutal, Moutal, Khanna & Khanna, 2017; Dustrude, Wilson, Ju, Xiao & Khanna, 2013). In the present disclosure, CBD3063 does not directly affect currents through sodium channels, or indirectly through an effect on CRMP2 phosphorylation or CRMP2 SUMOylation, indicating that this regulation is unaffected by CBD3063 treatment. These data, together with the lack of effect on K⁺ currents, suggest that targeting this interaction with CBD3063 does not result in inhibition of other ion channels relevant for pain signaling or in dysregulation of CRMP2 posttranslational modifications.

Despite the documented success of CBD3 peptide in achieving analgesia without side-effects, peptides are nevertheless hampered by (i) short half-life caused by their poor in vivo stability (Bruckdorfer, Marder & Albericio, 2004) which may be attributed to the presence of numerous peptidases and excretory mechanisms that inactivate and clear peptides, and (ii) negligible bioavailability caused by digestive enzymes that are designed to break down amide bonds of proteins and cleave the same bonds in these peptides. To circumvent some of these problems, in the present disclosure the pharmacophore modelling was utilized to generate small molecule peptidomimetics to improve upon the biological activity of the CBD3 peptide by mimicking the chemical features responsible for bioactivity with enhanced drug-like properties. As a result, in contrast to the short-lived actions of CBD3 peptides, the peptidomimetic developed here (i.e., CBD3063) exhibited prolonged antinociceptive profile following a single intrathecal administration as well as long-lasting (>14 days) reversal of mechanical allodynia with repeated intrathecal dosage in rats with chronic neuropathic pain. The latter also points to a lack of tolerance to CBD3063. The prolonged antinociceptive effect of CBD3063 could be attributed to the significant decrease of action potential firing in sensory neurons that could potentially translate into a decrease in spinal cord excitability and neurotransmitter release. These findings are congruent with those observed with an adenoviral form of a mutant CBD3 (i.e., AAV6-CBD3_{A6K}), which reduced the firing of dorsal horn neurons and reversed mechanical allodynia and thermal hyperalgesia for up to 6 weeks following intraganglionic delivery of the virus in rats with tibial nerve injury (Yu et al., 2019a).

The present disclosure provides a translational workflow that uses structural modeling to direct the resolution of protein-protein interactions involving poorly characterized disordered domains, resulting in both mechanistic insights and the identification of an analgesic. This translational workflow led to the discovery of CBD3063 as a first-in-class, CRMP2-based peptidomimetic, which selectively regulates Caᵥ2.2 to achieve analgesia. Although CBD3063 exhibited promising drug-likeness (QED score, Table 2) it does have marginal predicted blood-brain barrier penetration (BBB score, Table 2). To improve to CNS exposure, CBD3063 can be further optimized to lower the topological polar surface area by, for example, modifying and/or removing the acetamide and urea bonds. Another option to increase hydrophobicity is to introduce aromatic group (e.g., in the acetamide area) or to strategically position fluorine atoms to lower capacity of N—H donor (e.g., as 3-fluro-2-aminopyridyl moiety). Overall, targeted changes in CBD3063 are expected to improve its predicted ADME properties while maintaining its current biochemical profile.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

### REFERENCES

Abbadie et al., “Analgesic Effects of a Substituted N-Triazole Oxindole (TROX-1), a State-Dependent, Voltage-Gated Calcium Channel 2 Blocker,” J. Pharmacol. Exp. Ther. 334:545-555 (2010)
Alexander et al., “The Concise Guide to Pharmacology 2021/22: G Protein-Coupled Receptors,” British Journal of Pharmacology 178(Suppl 1):S27-S156 (2021)
Aparoy et al. “Structure and Ligand Based Drug Design Strategies in the Development of Novel 5-LOX Inhibitors,” Curr. Med. Chem. 19:3763-3778 (2012)
Attal et al., “EFNS Guidelines on Pharmacological Treatment of Neuropathic Pain,” Eur. J. Neurol. 13:1153-1169 (2006)
Basbaum et al., “Cellular and Molecular Mechanisms of Pain,” Cell 139:267-284 (2009)
Bauer et al., “The Anti-Allodynic Alpha(2)Delta Ligand Pregabalin Inhibits the Trafficking of the Calcium Channel Alpha(2)Delta-1 Subunit to Presynaptic Terminals in Vivo,” Biochem. Soc. Trans. 38:525-528 (2010)
Bell et al., “Cell-Specific Alternative Splicing Increases Calcium Channel Current Density in the Pain Pathway,” Neuron 41:127-138 (2004)
Bellampalli et al., “Betulinic Acid, Derived from the Desert Lavender Hyptis Emoryi, Attenuates Paclitaxel-, HIV-, and Nerve Injury-Associated Peripheral Sensory Neuropathy Via Block of N- and T-Type Calcium Channels,” Pain 160:117-135 (2019)
Bickerton et al., “Quantifying the Chemical Beauty of Drugs,” Nat. Chem. 4:90-98 (2012)
Brittain et al., “Neuroprotection Against Traumatic Brain Injury by a Peptide Derived from the Collapsin Response Mediator Protein 2 (CRMP2),” J. Biol. Chem. 286:37778-37792 (2011a)
Brittain et al., “Suppression of Inflammatory and Neuropathic Pain by Uncoupling CRMP-2 from the Presynaptic Ca(2)(+) Channel Complex,” Nat. Med. 17:822-829 (2011b)
Brittain et al., “An Atypical Role for Collapsin Response Mediator Protein 2 (CRMP-2) in Neurotransmitter Release Via Interaction with Presynaptic Voltage-Gated Calcium Channels,” J. Biol. Chem. 284:31375-31390 (2009)
Brittain et al., “Cdk5—Mediated Phosphorylation of CRMP-2 Enhances its Interaction with CaV2.2,” FEBS Lett. 586:3813-3818 (2012)
Bruckdorfer et al., “From Production of Peptides in Milligram Amounts for Research to Multi-Tons Quantities for Drugs of the Future,” Curr. Pharm. Biotechnol. 5:29-43 (2004)
Brustovetsky et al., “Collapsin Response Mediator Protein 2 (CRMP2) Interacts with N-Methyl-D-Aspartate (NMDA) Receptor and Na⁺/Ca²⁺ Exchanger and Regulates Their Functional Activity,” The Journal of Biological Chemistry 289:7470-7482 (2014)
Cai et al., “Activity of T-Type Calcium Channels is Independent of CRMP2 in Sensory Neurons,” Channels (Austin) 13:147-152 (2019)
Case et al., AMBER 2018 University of California, San Francisco (2018)
Chaplan et al., “Quantitative Assessment of Tactile Allodynia in the Rat Paw,” J. Neurosci. Methods. 53:55-63 (1994)
Chi et al., “Regulation of N-Type Voltage-Gated Calcium Channels (Caᵥ2.2) and Transmitter Release by Collapsin Response Mediator Protein-2 (CRMP-2) in Sensory Neurons,” J. Cell Sci. 122:4351-4362 (2009)
Choe et al., “TTA-P2 is a Potent and Selective Blocker of T-Type Calcium Channels in Rat Sensory Neurons and a Novel Antinociceptive Agent,” Mol. Pharmacol. 80:900-910 (2011)
Cizkova et al., “Localization of N-Type Ca²⁺ Channels in the Rat Spinal Cord Following Chronic Constrictive Nerve Injury,” Exp. Brain Res. 147:456-463 (2002)
Decosterd I, & Woolf C J., “Spared Nerve Injury: An Animal Model of Persistent Peripheral Neuropathic Pain,” Pain 87:149-158 (2000)
Doggrell S A, “Intrathecal Ziconotide for Refractory Pain,” Expert Opin. Investig. Drugs 13:875-877 (2004)
Dustrude et al., “Hierarchical CRMP2 Posttranslational Modifications Control NaV1.7 Function,” Proc. Natl. Acad. Sci. 113:E8443-E8452 (2016)
Dustrude et al., “A Single Structurally Conserved SUMOylation Site in CRMP2 Controls NaV1.7 Function,” Channels (Austin) 11:316-328 (2017)
Dustrude et at., “CRMP2 Protein SUMOylation Modulates NaV1.7 Channel Trafficking,” The Journal of Biological Chemistry 288:24316-24331 (2013)
Evans et al., “Differential Regulation of Evoked Peptide Release by Voltage-Sensitive Calcium Channels in Rat Sensory Neurons,” Brain Res. 712:265-273(1996)
Evoy et al., “Gabapentinoid Pharmacology in the Context of Emerging Misuse Liability,” Journal of Clinical Pharmacology 61(Suppl 2):S89-S99 (2021)
Feng et al., “Residue Gly1326 of the N-Type Calcium Channel Alpha 1B Subunit Controls Reversibility of Omega-Conotoxin GVIA and MVIIA Block,” J. Biol. Chem. 276:15728-15735 (2001)
Fischer et al., “Sustained Relief of Neuropathic Pain by AAV-Targeted Expression of CBD3 Peptide in Rat Dorsal Root Ganglion,” Gene Ther. 21:44-51 (2014)
Fosgerau K, & Hoffmann T., “Peptide Therapeutics: Current Status and Future Directions,” Drug Discov. Today 20:122-128 (2015)
Francois-Moutal et al., “A Membrane-Delimited N-Myristoylated CRMP2 Peptide Aptamer Inhibits CaV2.2 Trafficking and Reverses Inflammatory and Postoperative Pain Behaviors,” Pain 156:1247-1264 (2015)
Giglio et al., “Behavioral and Physiological Methods for Early Quantitative Assessment of Spinal Cord Injury and Prognosis in Rats,” Braz. J. Med. Biol. Res. 39:1613-1623 (2006)
Gomez et al., “Stereospecific Effects of Benzimidazolonepiperidine Compounds on T-Type Ca(2+) Channels and Pain,” ACS Chem. Neurosci. 13(13):2035-2047 (2022)
Goshima et al., “Collapsin-Induced Growth Cone Collapse Mediated by an Intracellular Protein Related to UNC-33,” Nature 376:509-514 (1995)
Götz et al., “Routine Microsecond Molecular Dynamics Simulations with AMBER on GPUs. 1.
Generalized Born,” J. Chem. Theory Comput. 8:1542-1555 (2012)
Gupta et al., “The Blood-Brain Barrier (BBB) Score,” J. Med. Chem. 62:9824-9836 (2019)
Hatakeyama et al., “Differential Nociceptive Responses in Mice Lacking the Alpha(1B) Subunit of N-Type Ca(2+) Channels,” Neuroreport 12:2423-2427 (2001)
Heinke et al., “Pre- and Postsynaptic Contributions of Voltage-Dependent Ca²⁺ Channels to Nociceptive Transmission in Rat Spinal Lamina I Neurons,” Eur. J. Neurosci. 19:103-111 (2004)
Hendrich et al., “Pharmacological Disruption of Calcium Channel Trafficking by the Alpha2delta Ligand Gabapentin,” Proceedings of the National Academy of Sciences of the United States of America 105:3628-3633 (2008)
Hoppanova L, & Lacinova L., “Voltage-Dependent CaV3.2 and CaV2.2 Channels in Nociceptive Pathways,” Pflugers Arch. 474:421-434 (2022)
Inagaki et al., “CRMP-2 Induces Axons in Cultured Hippocampal Neurons,” Nat. Neurosci. 4:781-782 (2001)
Irwin et al., “The Effects of Morphine Methadone and Meperidine on Some Reflex Responses of Spinal Animals to Nociceptive Stimulation,” J. Pharmacol. Exp. Ther. 101:132-143 (1951)
Ji et al. “CRMP2-Derived Peptide ST2-104 (R9-CBD3) Protects SH-SY5Y Neuroblastoma Cells Against Abeta25-35-Induced Neurotoxicity by Inhibiting the pCRMP2/NMDAR2B Signaling Pathway,” Chem. Biol. Interact. 305:28-39 (2019)
Ju et al., “Suppression of Pain-Related Behavior in Two Distinct Rodent Models of Peripheral Neuropathy by a Homopolyarginine-Conjugated CRMP2 Peptide,” J. Neurochem. 124:869-879 (2013)
Khanna et al., “Targeting the CaValpha-CaVbeta Interaction Yields an Antagonist of the N-type CaV2.2 Channel with Broad Antinociceptive Efficacy,” Pain 160:1644-1661 (2019)
Khanna et al., “A Proteomic Screen for Presynaptic Terminal N-Type Calcium Channel (CaV2.2) Binding Partners,” J. Biochem. Mol. Biol. 40:302-314 (2007)
Kim et al., “Altered Nociceptive Response in Mice Deficient in the Alpha(1B) Subunit of the Voltage-Dependent Calcium Channel,” Mol. Cell Neurosci. 18:235-245 (2001)
Koes D R, & Camacho C J., “ZINCPharmer: Pharmacophore Search of the ZINC Database,” Nucleic Acids Res. 40:W409-414 (2012)
Kozakov et al., “Optimal Clustering for Detecting Near-Native Conformations in Protein Docking,” Biophys. J. 89:867-875 (2005)
Kuehn B M., “Gabapentin Increasingly Implicated in Overdose Deaths,” JAMA 327:2387 (2022)
Li et al., “Up-Regulation of Cavbeta3 Subunit in Primary Sensory Neurons Increases Voltage-Activated Ca2+ Channel Activity and Nociceptive Input in Neuropathic Pain,” J. Biol. Chem. 287:6002-6013 (2012)
Lipinski C A., “Lead- and Drug-Like Compounds: The Rule-of-Five Revolution,” Drug Discov. Today Technol. 1:337-341 (2004)
Ludwig et al., “Regional Expression and Cellular Localization of the Alpha1 and Beta Subunit of High Voltage-Activated Calcium Channels in Rat Brain,” J. Neurosci. 17:1339-1349 (1997)
Maier et al., “ff14SB: Improving the Accuracy of Protein Side Chain and Backbone Parameters from ff99SB,” J. Chem. Theory Comput. 11:3696-3713 (2015)
Mintz et al., “P-Type Calcium Channels Blocked by the Spider Toxin Omega-Aga-IVA,” Nature 355:827-829 (1992)
Moutal et al. “Homology-Guided Mutational Analysis Reveals the Functional Requirements for Antinociceptive Specificity of Collapsin Response Mediator Protein 2-Derived Peptides,” Br. J. Pharmacol. 175:2244-2260 (2018)
Murakami et al., “Pain Perception in Mice Lacking the Beta3 Subunit of Voltage-Activated Calcium Channels,” J. Biol. Chem. 277:40342-40351 (2002)
Newcomb et al. “Selective Peptide Antagonist of the Class E Calcium Channel from the Venom of the Tarantula Hysterocrates Gigas,” Biochemistry 37:15353-15362 (1998)
Nieto-Rostro et al., “Ablation of Alpha2delta-1 Inhibits Cell-Surface Trafficking of Endogenous N-Type Calcium Channels in the Pain Pathway in Vivo,” Proc. Natd. Acad. Sci. 115:E12043-E12052 (2018)
Nowycky et al., “Three Types of Neuronal Calcium Channel with Different Calcium Agonist Sensitivity,” Nature 316:440-443 (1985)
Perez J J., “Designing Peptidomimetics,” Curr. Top Med. Chem. 18:566-590 (2018)
Piekarz et al. “CRMP-2 Peptide Mediated Decrease of High and Low Voltage-Activated Calcium Channels, Attenuation of Nociceptor Excitability, and Anti-Nociception in a Model of AIDS Therapy-Induced Painful Peripheral Neuropathy,” Mol. Pain 8: 54 (2012)
Rajamani et al., “Anchor Residues in Protein-Protein Interactions,” Proc. Natl. Acad. Sci. USA 101:11287-11292(2004)
Richards et al., “Ca2+ Channel Beta-Subunits: Structural Insights AID our Understanding,” Trends Pharmacol. Sci. 25:626-632 (2004)
Ripsch et al., “A Peptide Uncoupling Crmp-2 From the Presynaptic Ca(2+) Channel Complex Demonstrates Efficacy in Animal Models of Migraine and Aids Therapy-Induced Neuropathy,” Transl. Neurosci. 3:1-8 (2012)
Salomon-Ferrer et al., “Routine Microsecond Molecular Dynamics Simulations with AMBER on GPUs. 2. Explicit Solvent Particle Mesh Ewald,” J. Chem. Theory Comput. 9:3878-3888 (2013)
Scott et al., “Beta Subunit Heterogeneity in N-Type Ca2+ Channels,” J. Biol. Chem. 271:3207-3212 (1996)
Shao et al., “Aminopiperidine Sulfonamide Cav2.2 Channel Inhibitors for the Treatment of Chronic Pain,” J. Med. Chem. 55:9847-9855 (2012)
Smith A J., “New Horizons in Therapeutic Antibody Discovery: Opportunities and Challenges Versus Small-Molecule Therapeutics,” J. Biomol. Screen. 20:437-453 (2015)
Sterling T, & Irwin J J., “ZINC 15—Ligand Discovery for Everyone,” J. Chem. Inf Model 55:2324-2337 (2015)
Striessnig J., “Getting a Handle on Ca(V)2.2 (N-type) Voltage-Gated Ca(2+) Channels,” Proc. Natd. Acad. Sci. USA 115:12848-12850 (2018)
Sutton et al., “Gabapentin Inhibits High-Threshold Calcium Channel Currents in Cultured Rat Dorsal Root Ganglion Neurones,” Br. J. Pharmacol. 135:257-265 (2002)
Welling et al., “Stable Co-Expression of Calcium Channel Alpha 1, Beta and Alpha 2/delta Subunits in a Somatic Cell Line,” J. Physiol 0.471:749-765 (1993)
Xie et al., “Sustained Relief of Ongoing Experimental Neuropathic Pain by a CRMP2 Peptide Aptamer With Low Abuse Potential,” Pain 157:2124-2140 (2016)
Yaksh T L, & Rudy T A., “Chronic Catheterization of the Spinal Subarachnoid Space,” Physiol. Behav. 17:1031-1036 (1976)
Yang et al., “Upregulation of N-Type Calcium Channels in the Soma of Uninjured Dorsal Root Ganglion Neurons Contributes to Neuropathic Pain by Increasing Neuronal Excitability Following Peripheral Nerve Injury,” Brain Behav. Immun. 71:52-65 (2018)
Yeh et al., “Defining the Kv2.1-Syntaxin Molecular Interaction Identifies a First-in-Class Small Molecule Neuroprotectant,” Proc. Natl. Acad. Sci. USA 116:15696-15705 (2019)
Yu et al. “AAV-Encoded Ca(V)2.2 Peptide Aptamer CBD3A6K for Primary Sensory Neuron-Targeted Treatment of Established Neuropathic Pain,” Gene Ther. 26:308-323 (2019a)
Yu et al., “Phosphorylated CRMP2 Regulates Spinal Nociceptive Neurotransmission,” Mol. Neurobiol. 56:5241-5255 (2019b)
Zamponi G W., “Regulation of Presynaptic Calcium Channels by Synaptic Proteins,” J. Pharmacol. Sci. 92:79-83 (2003)
Zamponi et al., “The Physiology, Pathology, and Pharmacology of Voltage-Gated Calcium Channels and Their Future Therapeutic Potential,” Pharmacological Reviews 67:821-870 (2015)
Zheng et al., “Tuning Microtubule Dynamics to Enhance Cancer Therapy by Modulating FER-Mediated CRMP2 Phosphorylation,” Nat. Commun. 9:476 (2018)

All patents, applications, publications, test methods, literature, and other materials cited herein are hereby incorporated by reference in their entirety as if physically present in this specification.

The invention claimed is:

1. A compound of Formula (I): 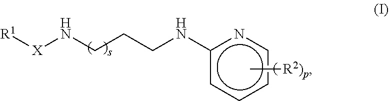
wherein
X is a covalent bond or —C(O)—;
p is 0, 1, 2, 3, or 4;
s is 1 or 2;
R¹ is selected from the group consisting of C₁₋₆ alkyl, benzyl, —NR³R⁴, monocyclic or bicyclic heteroaryl, and monocyclic or bicyclic heterocyclyl, wherein C₁₋₆ alkyl, benzyl, monocyclic or bicyclic heteroaryl, and monocyclic or bicyclic heterocyclyl can be optionally substituted from 1 to 3 times with R⁵;
R² is independently at each occurrence a C₁₋₆ alkyl;
R³ is H;
R⁴ is selected from the group consisting of —H, C₁₋₆ alkyl, C₃₋₈ cycloalkyl, monocyclic or bicyclic aryl, hereroaryl, heterocyclyl, —CH₂-heterocyclyl, and —CH₂-heteroaryl, wherein C₁₋₆ alkyl, monocyclic or bicyclic aryl, heteroaryl, heterocyclyl, —CH₂-heterocyclyl, and —CH₂-heteroaryl can be optionally substituted from 1 to 3 times with R⁶; or
R³ and R⁴ combine with the nitrogen atom to which they are attached to form a non-aromatic heterocyclyl, wherein the non-aromatic heterocyclyl can be optionally substituted from 1 to 3 times with a substituent selected independently from the group consisting of —H, C₁₋₆ alkyl, and —NH—C(O)—Me;
R⁵ is selected from the group consisting of —H, halogen, —OH, phenyl, C₁₋₆ alkyl, benzyl, —OC₁₋₆ alkyl, ═O, C₃₋₈ cycloalkyl, monocyclic heteroaryl, wherein C₁₋₆ alkyl and C₃₋₈ cycloalkyl can be optionally substituted from 1 to 3 times with —NH₂;
R⁶ is independently selected from the group consisting of —H, —NMe₂, C₁₋₆ alkyl, —O—C₁₋₆ alkyl, heteroaryl, —CH₂-heterocyclyl, —CH₂-heteroaryl, heterocyclyl, —OPh, C₃₋₈ cycloalkyl, ═O, and heteroaryl, wherein —CH₂-heterocyclyl, —CH₂-heteroaryl, heterocyclyl, heteroaryl, and —OPh can be optionally substituted from 1 to 3 times with a substituent selected independently from the group consisting of —H, C₁₋₆ alkyl, C₃₋₈ cycloalkyl, —OH, ═O, halogen, and —SC₁₋₆ alkyl,
or a solvate, or a pharmaceutically acceptable salt thereof.

2. (canceled)

3. The compound of claim 1, wherein the compound of Formula (I) has a Formula (IA) or Formula (IB):

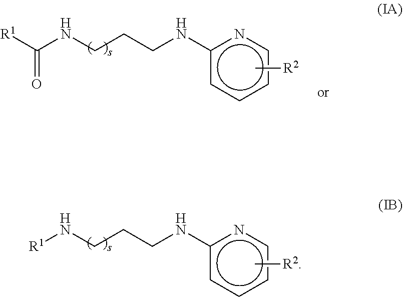

4. (canceled)

5. The compound of claim 1, wherein R¹ is selected from the group consisting of

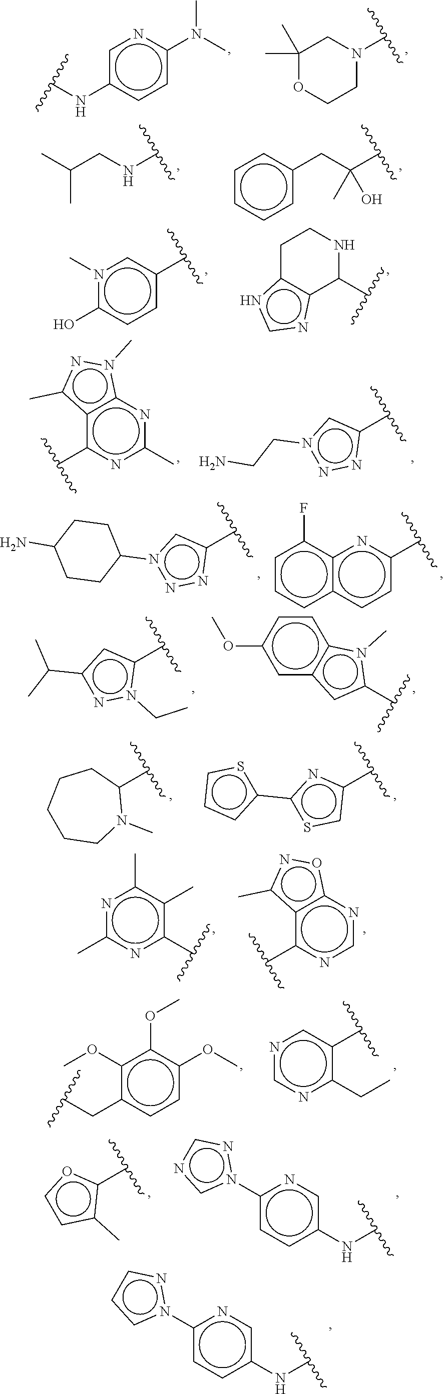

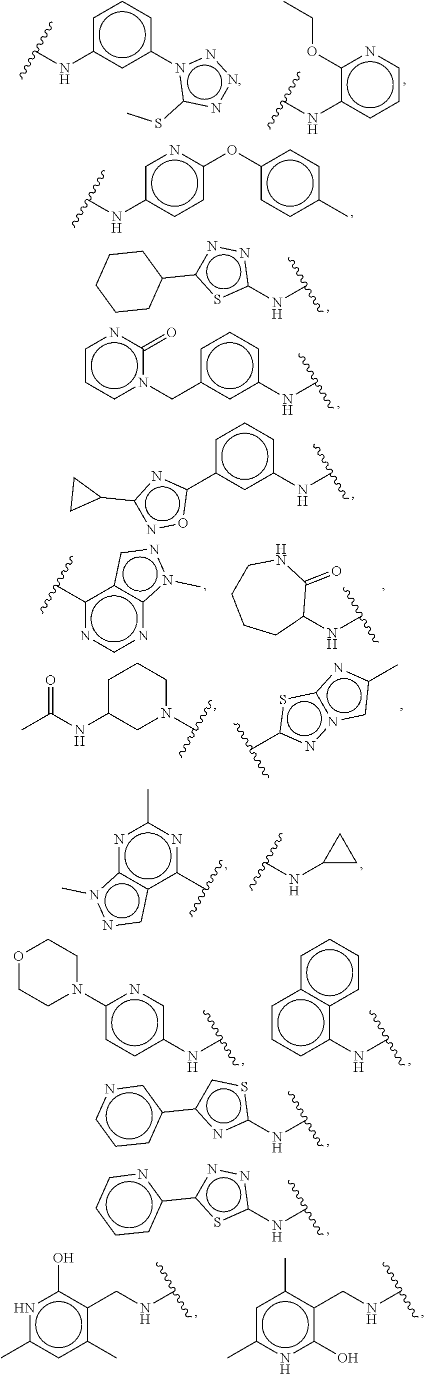

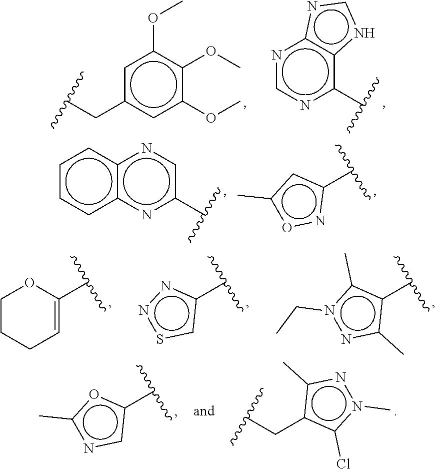

6. The compound of claim 1, wherein R³ and R⁴ combine with the nitrogen atom to which they are attached to form morpholinyl or piperidinyl, wherein the morpholinyl or the piperidinyl can be optionally substituted from 1 to 3 times with H, C₁₋₆ alkyl, or —NH—C(O)—Me.

7. (canceled)

8. The compound of claim 1, wherein the compound of Formula (I) is selected from a group consisting of

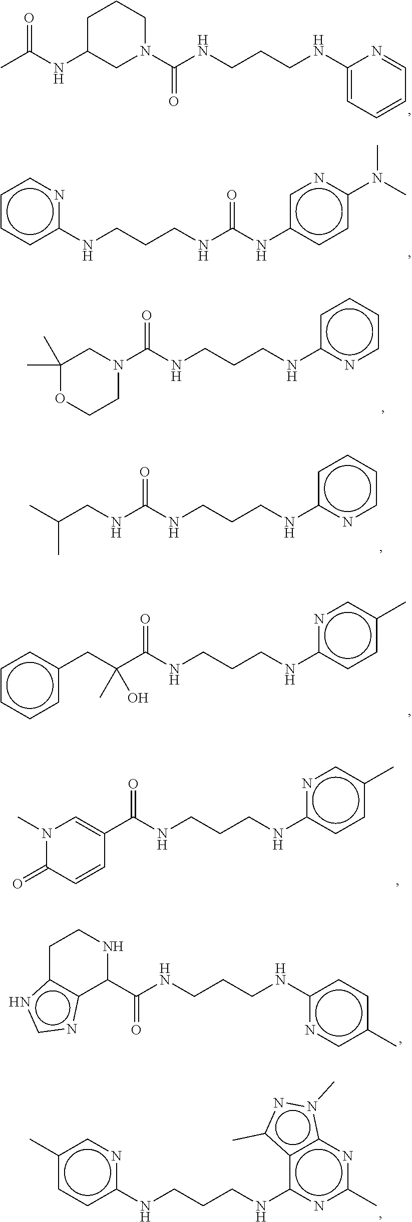

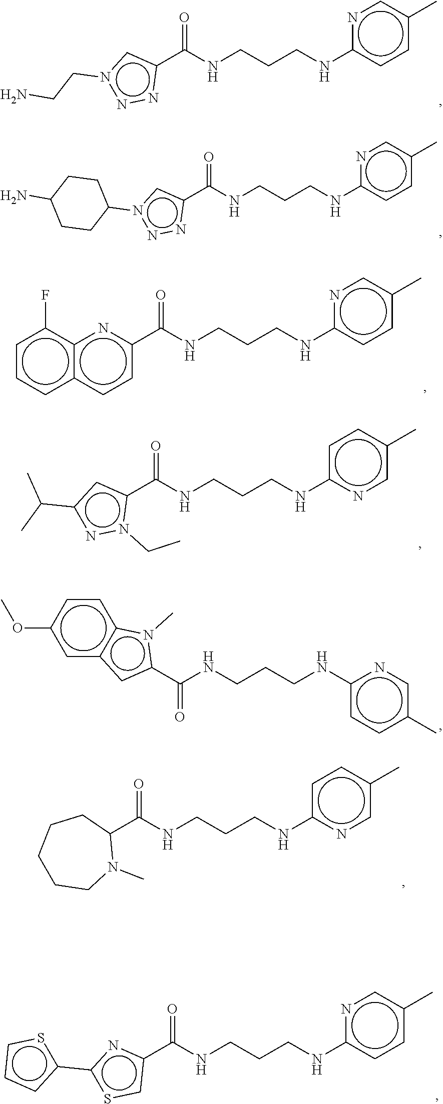

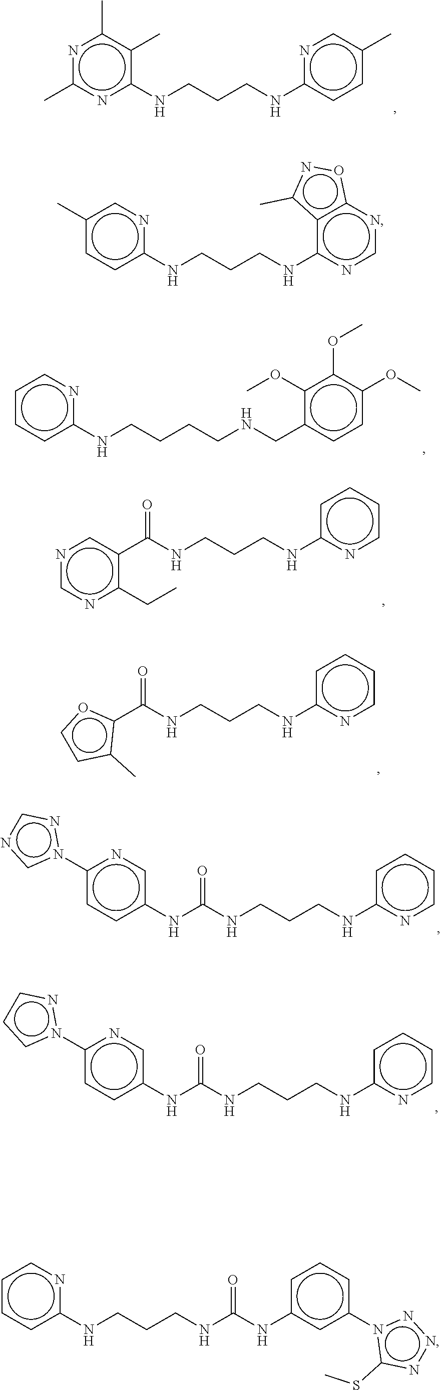

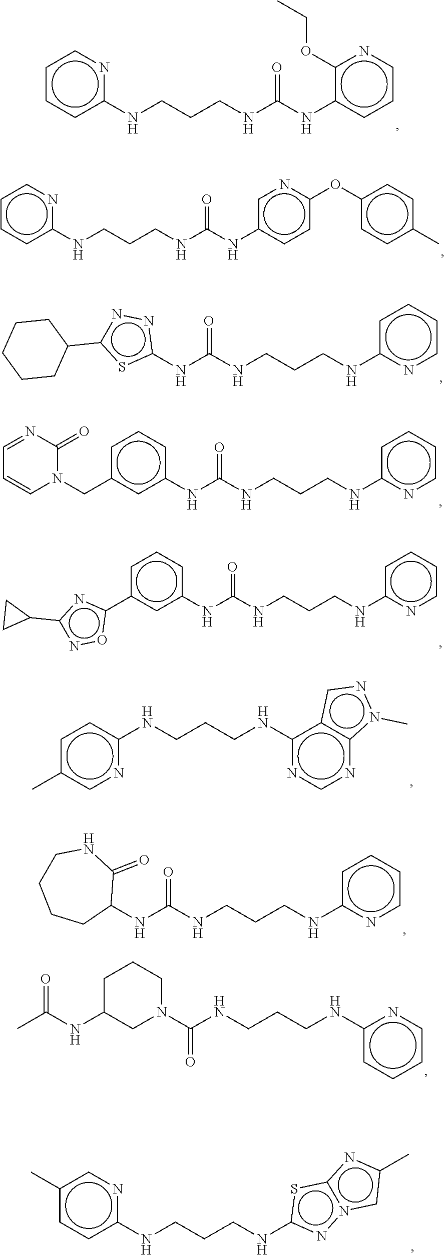

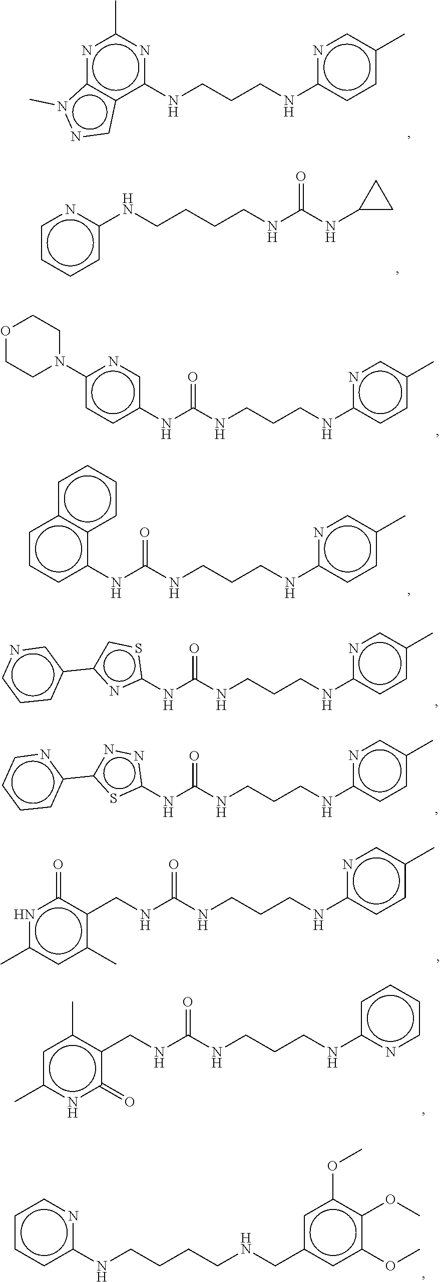

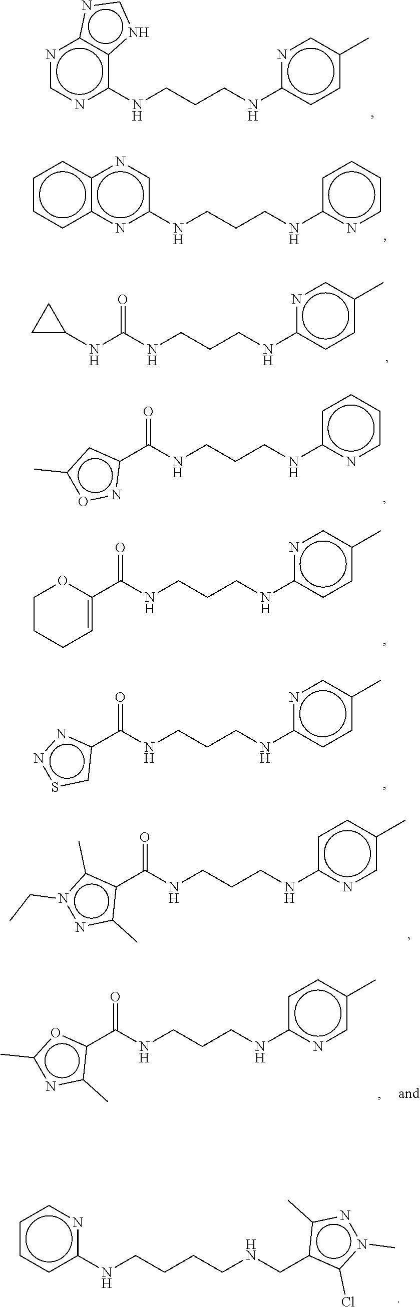

9. A compound of Formula (II): 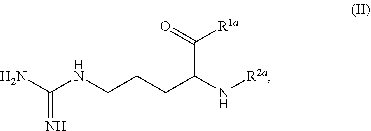
wherein
R^{1a} is selected from the group consisting of —OH, —OC₁₋₆ alkyl, and —NHR^{3a};
R^{2a} is selected from the group consisting of —H, —C(O)—R^{4a}, —C(Y)C₁₋₆ alkyl, —C(Y)C₃₋₈ cycloalkyl, —C(O)OC₁₋₆ alkyl, —C(Y)aryl, —C(Y)NH-aryl, —C(Y)NH—CH₂-aryl, —C(Y)NHC₃₋₈ cycloalkyl, and —S(O)₂aryl, wherein —C(O)—R^{4a}, —C(Y)C₁₋₆ alkyl, —C(Y)C₃₋₈ cycloalkyl, —C(O)OC₁₋₆ alkyl, —C(Y)aryl, —C(Y)NH-aryl, —C(Y)NH—CH₂-aryl, —C(Y)NHC₃₋₈ cycloalkyl, and —S(O)₂aryl can be optionally substituted from 1 to 3 times with a substituent selected independently from C₁₋₆ alkyl and —NH₂,
R^{3a} is selected from C₁₋₆ alkyl or monocyclic or bicyclic aryl, wherein C₁₋₆ alkyl and monocyclic and bicyclic aryl can be optionally substituted from 1 to 3 times with a substituent selected independently from —COOH and —NO₂;
R^{4a} is non-aromatic heterocyclyl optionally substituted with —C(O)OC₁₋₆ alkyl;
Y is O or S,
or a solvate, or a pharmaceutically acceptable salt thereof.

10. The compound of claim 9, wherein R^{1a} is selected from the group consisting of —OH, —OMe, —OEt,

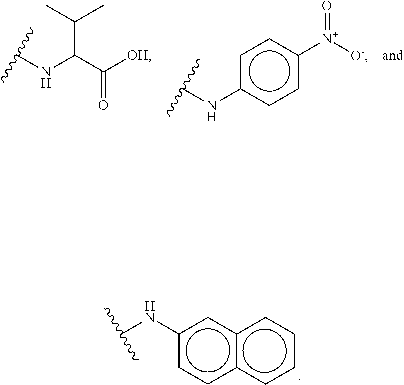

11. The compound of claim 9, wherein
R^{2a} is selected from the group consisting of —H,

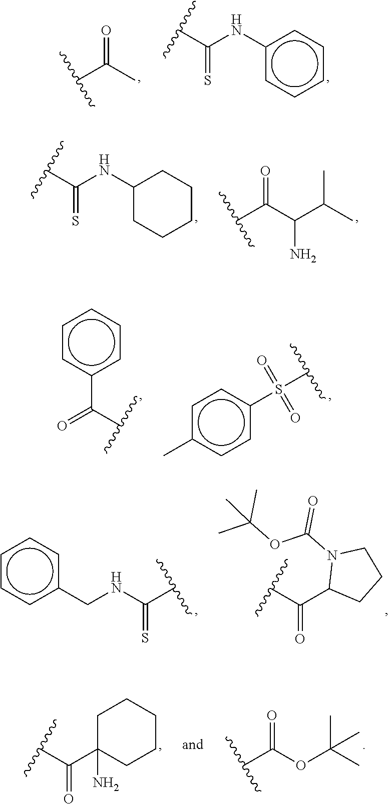

12. The compound of claim 9, wherein the compound of Formula (II) is selected from a group consisting of

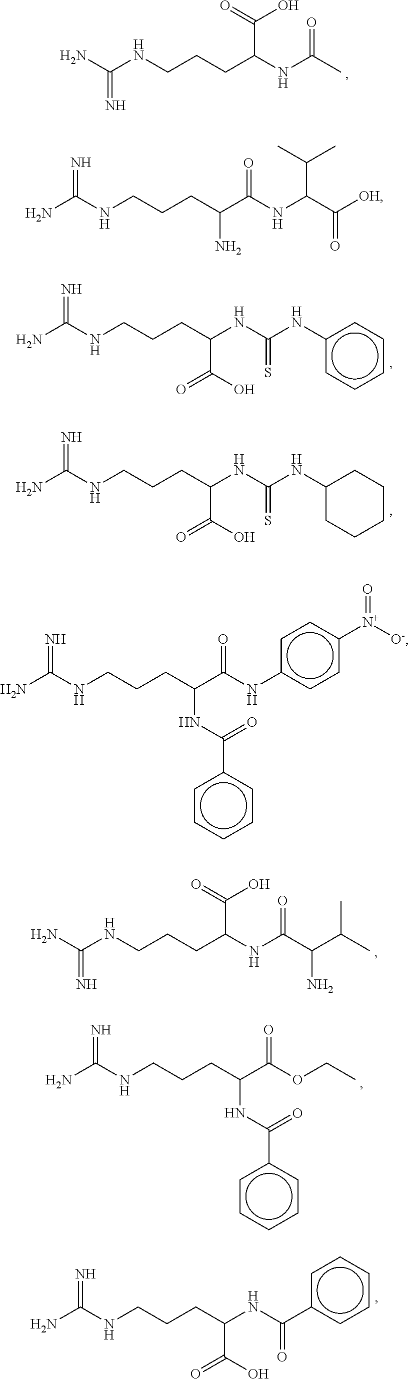

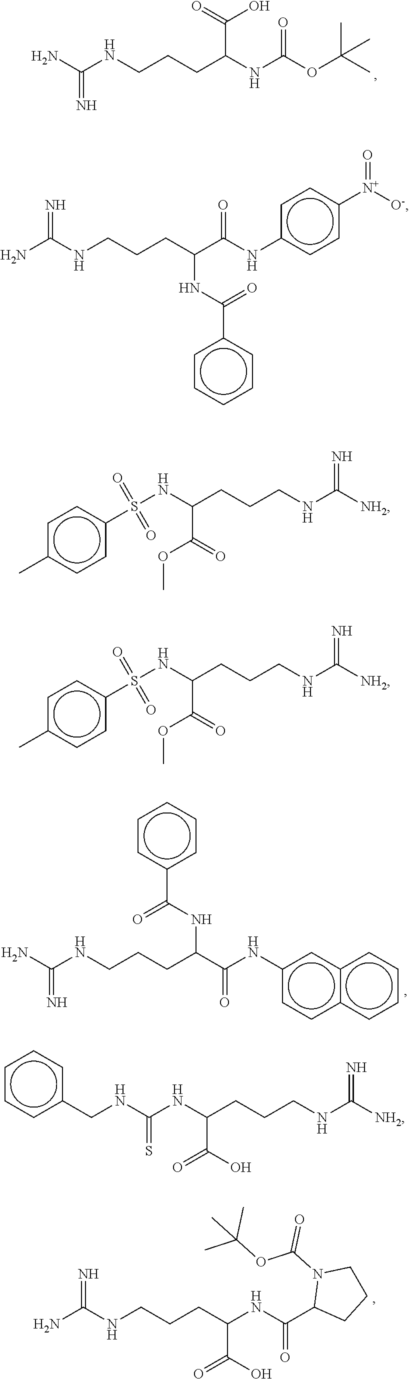

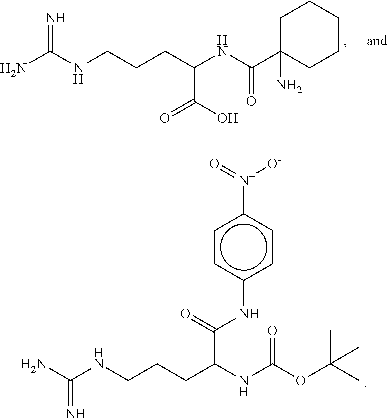

13. A compound of Formula (III): 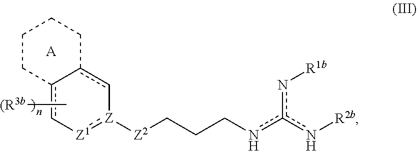 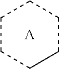
is optional and if present is 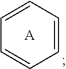 [symbol]
is a single or a double bond;
Z is C or N;
Z¹ is CH, CH₂, N, or a covalent bond;
Z² is CH₂ or a covalent bond;
n is 0, 1, or 2;
R^{1b} is selected from the group consisting of —H, C₁₋₆ alkyl, C₃₋₈ cycloalkyl, aryl, and benzyl, wherein C₁₋₆ alkyl, aryl, and benzyl can be optionally substituted with a substituent selected from —CN, —OC₁₋₆ alkyl, heteroaryl, and non-aromatic heterocyclyl;
R^{2b} is selected from the group consisting of —H, C₁₋₆ alkyl, C₃₋₈ cycloalkyl, aryl, and benzyl, wherein C₁₋₆ alkyl, aryl, and benzyl can be optionally substituted with a substituent selected from —CN, —OC₁₋₆ alkyl, heteroaryl, and non-aromatic heterocyclyl;
R^{3b} is —H or C₁₋₆ alkyl,
or a solvate, or a pharmaceutically acceptable salt thereof.

14.-21. (canceled)

22. A compound of Formula (IV): 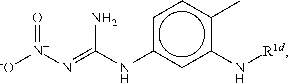
wherein
R^{1d} is —COOC₁₋₆ alkyl, —C(O)aryl, or —C(O)heteroaryl, wherein —C(O)aryl can be optionally substituted with —NO₂,
or a solvate, or a pharmaceutically acceptable salt thereof.

23-24. (canceled)

25. A compound selected from the group consisting of 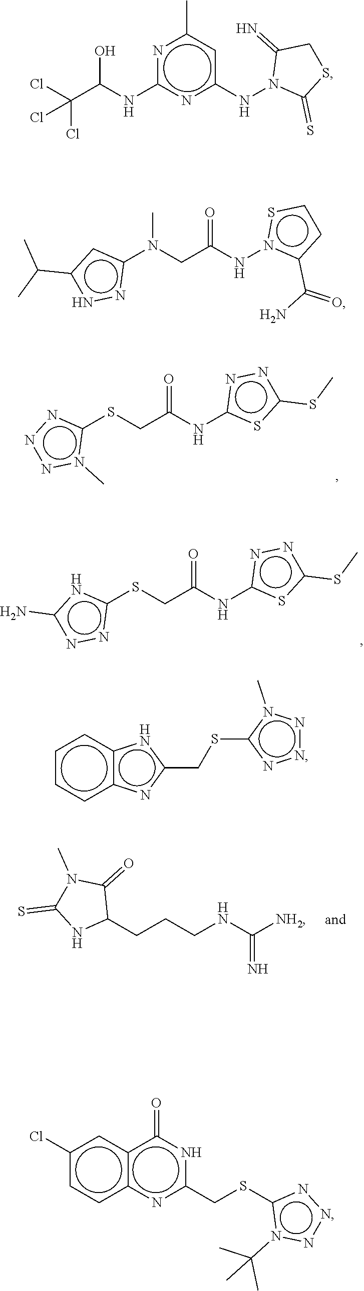
or a solvate, or a pharmaceutically acceptable salt thereof.

26. A pharmaceutical composition comprising the compound according to claim 1.

27. A pharmaceutical dosage form comprising the compound according to claim 1.

28. A method of regulating Cav2.2, the method comprising contacting Cav2.2 with the compound according to claim 1.

29. The method of claim 28, wherein the regulating Cav2.2 is inhibiting Cav2.2.

30. The method of claim 28, wherein the regulating Cav2.2 is reducing membrane expression of Cav2.2.

31. The method of claim 28, wherein the regulating Cav2.2 is uncoupling the Cav2.2-CRMP2 interaction.

32. A method of treating, reducing or alleviating pain in an individual in need thereof comprising administering to the individual an effective amount of the compound according to claim 1.

33. A method of identifying one or more compounds capable of regulating Cav2.2 comprising:
providing a CBD3 peptide;
identifying one or more stable structural motif(s) of the CBD3 peptide;
providing one or more pharmacophore models that map the stable structural motif of the CBD3 peptide or it's properties into said pharmacophore models;
providing one or more compounds;
screening each of said pharmacophore models against said compounds; and
selecting one or more compounds that have a highest Cav2.2 recognition score for future testing.
